(19) United States
(12) Patent Application Publication
(10) Pub. No.: US 2020/0078466 A1
(43) Pub. Date: Mar. 12, 2020

(54) GLYCOPEPTIDE ANTIBIOTIC CONSTRUCTS

(71) Applicant: THE UNIVERSITY OF QUEENSLAND, St Lucia, Queensland (AU)

(72) Inventors: Mark BLASKOVICH, St Lucia, Queensland (AU); Matt COOPER, Cambridge (GB); Andrea RANZONI, Sydney (AU); Wanida PHETSANG, Pathumwan, Bangkok (TH); Marwa HASSAN, Coventry (GB)

(21) Appl. No.: 16/467,634

(22) PCT Filed: Dec. 11, 2017
(86) PCT No.: PCT/AU2017/051364

§ 371 Date: Jun. 7, 2019

(30) Foreign Application Priority Data
Dec. 9, 2016 (AU) 2016905104

(51) Int. Cl. A61K 47/55; A61K 38/14; A61K 47/61; A61K 47/62; C12N 1/02; A61K 47/69; C07K 17/14; C08B 37/02; C09D 105/02; C12Q 1/04; C12Q 1/24; G01N 33/569

(52) U.S. Cl. CPC A61K 47/552; A61K 38/14; A61K 47/61; A61K 47/62; C12N 1/02; A61K 47/6923; G01N 33/56911; C07K 17/14; C08B 37/0021; C09D 105/02; C12Q 1/04; C12Q 1/24; A61K 47/6929

(57) ABSTRACT

A construct comprising: (i) an optionally derivatized glycopeptide antibiotic; (ii) a nanoparticle; and (iii) a first linker connecting (i) and (ii) is provided. The construct may further comprise a second linker located between the first linker and (ii). The nanoparticle may be a separation nanoparticle, such as a magnetic separation nanoparticle. The glycopeptide antibiotic may be selected from the group consisting: of vancomycin; teicoplanin; oritavancin; telavancin; chloroeremomycin; and balhimycin. Also provided are related methods of producing and using the construct, such as methods of separation of bacteria from a sample by binding the bacteria to the construct.

### TECHNICAL FIELD

THE present invention relates to constructs with microbial binding activity. In some forms, the invention relates to constructs for bioseparation of gram positive bacteria, and associated methods of bacterial analysis and/or disease diagnosis. The invention also relates to constructs with antimicrobial activity, and use of such constructs for microbial control.

### BACKGROUND

Microorganisms such as bacteria cause a vast range of diseases or conditions of living organisms, including plants and animals such as humans. Since the middle of last century, antibiotics have been effectively used for control of bacterial diseases. However, bacteria have substantial capacity to develop resistance to antibiotics, and disease caused by antibiotic resistant bacteria is currently considered a major global crisis.

Bacteraemia and sepsis (or septicaemia) are related conditions in which microorganisms (typically bacteria) are present in blood. In bacteraemia, a microorganism enters the blood, e.g. through a wound, infection, or surgical procedure. In sepsis, a microorganism enters and replicates in the blood. Sepsis is typically considered a very serious condition, and is frequently life-threatening. Sepsis is commonly caused by the Gram positive cocci bacteria Staphylococcus species, particularly S. aureus. However, other Gram positive bacteria including Streptococcus, and Enterococcus species can cause sepsis, as can certain Gram negative bacteria (e.g. Escherichia, Pseudomonas, and Klebsiella species) and fungi, e.g. Candida species.

For patients presenting with sepsis, the time taken to identify the causative pathogen and to start administering the correct antibiotic is critical. Current methods for detecting microorganisms in the blood (e.g. bacteraemia) are typically culture-based. However these methods may be affected by a high rate of false positives and a relatively lengthy time for diagnosis. In addition, hospitals face substantial issues caused by hospital acquired infections and multi-drug resistant bacteria (e.g. Methicillin-Resistant Staphylococcus aureus; MRSA) in the context of sepsis and other infections, with such patients requiring longer stays, more oversight and use of expensive last line antibiotics.

In consideration of the above, new approaches to microorganism detection that are suited for diagnosis of bacteraemia and/or sepsis are highly desirable. Furthermore, strategies for enhancing the antimicrobial activity of antibiotics would be of great benefit.

### SUMMARY

In one broad form, the present invention is directed to constructs comprising an optionally derivatized glycopeptide antibiotic connected to a nanoparticle.

In a first aspect of this first broad form, there is provided a construct comprising:
(i) an optionally derivatized glycopeptide antibiotic;
(ii) a nanoparticle; and
(iii) a first linker connecting (i) and (ii).

Constructs of this aspect may have the following general structure:

A-L₁-N;
wherein:
A is the optionally derivatized glycopeptide antibiotic;
L₁ is the first linker; and
N is the nanoparticle.

The connections between A, L₁, and N may be direct connections or indirect connections.

In a preferred embodiment of the first aspect, the construct comprises:
(i) an optionally derivatized glycopeptide antibiotic;
(ii) a nanoparticle;
(iii) a first linker connected to (i); and
(iv) a second linker connected to (ii),

wherein (iii) is connected to (iv) and (i) is connected to (ii).

Constructs of this embodiment may have the following general structure:

A-L₁-L₂-N
wherein:
A is the optionally derivatized glycopeptide antibiotic;
L₁ is the first linker;
L₂ is the second linker; and
N is the nanoparticle.

The connections between A and L₁; L₁ and L₂; and L₂ and N may be direct or indirect connections. In preferred embodiments, the connection between A and L₁ is a direct connection. In preferred embodiments, the connection between L₁ and L₂ is a direct connection. In preferred embodiments, the connection between L₂ and N is an indirect connection.

Suitably, the construct of the first aspect is for binding to a microorganism or a component thereof, wherein the optionally derivatized glycopeptide antibiotic binds to the microorganism or component thereof.

Preferably, the microorganism is a Gram positive bacteria.

In some preferred embodiments the nanoparticle is a separation nanoparticle. Preferably, the separation nanoparticle is a magnetic nanoparticle.

In certain preferred embodiments, the optionally substituted glycopeptide antibiotic is selected from the group consisting: of vancomycin; teicoplanin; oritavancin; telavancin; chloroeremomycin; and balhimycin. In one particularly preferred embodiment, the glycopeptide antibiotic is vancomycin.

In some embodiments, the first linker of the construct is at least partially hydrophilic.

In preferred embodiments wherein the first linker is at least partially hydrophilic, it comprises a polyethylene glycol (PEG) moiety. Preferably, the PEG moiety comprises at least PEG3. In some particularly preferred embodiments, the PEG moiety is PEG3 or PEG4.

In some embodiments, the first linker of the construct is hydrophobic.

In some embodiments wherein the first linker is hydrophobic, the first linker comprises a linear carbon chain of greater than four carbons. Preferably the linear carbon chain is C4-C12. In one particularly preferred embodiment, the linear carbon chain is C8. In another particularly preferred embodiment, the linear carbon chain is C11.

In preferred embodiments, the first linker comprises one or more nitrogen-containing moieties. Preferably, the one or more nitrogen-containing moieties include an amine-derived moiety and/or an azide-derived moiety.

Preferably, the first linker comprises a nitrogen-containing moiety at a first end of the linker. Preferably, the moiety is an amine-derived moiety. Preferably the moiety connects the linker to the glycopeptide antibiotic. The nitrogen-containing moiety may be an amide bond formed between an amine group from a first end of a precursor to the first linker and the C-terminal carboxy moiety from the glycopeptide antibiotic.

In preferred embodiments, the first linker is connected to the nanoparticle via a second linker, as set forth above. Preferably, the second linker is an organic molecule. In these embodiments, preferably, the first linker comprises a nitrogen-containing moiety at a second end of the first linker. In preferred embodiments, this moiety is an azide-derived moiety. Preferably the first linker is connected to the second linker via the azide-derived moiety of the first linker. Preferably, the connection between the first linker and the second linker comprises a triazole moiety. Suitably, the triazole moiety is formed between an azide group from the second end of a precursor to the first linker and an alkyne group of a precursor to the second linker.

In some preferred embodiments, the second linker comprises a PEG group. Preferably, the PEG group is at least PEG3. In some particularly preferred embodiments, the PEG group is PEG3 or PEG4.

Preferably, the nanoparticle of the construct is passivated. In embodiments, the nanoparticle is passivated by a coating. Preferably the coating is a protein or a polymer coating.

In an embodiment, the separation nanoparticle is passivated with human serum albumin (HSA). In an embodiment, the separation nanoparticle is passivated by coating with carboxymethyl-PDEC-dextran (CMD). In an embodiment, the separation nanoparticle is passivated by coating with PDEC-dextran. In an embodiment, the separation nanoparticle is passivated by coating with PDEA-dextran.

Preferably, in embodiments wherein the separation nanoparticle is passivated by coating, the first linker is connected to the nanoparticle via the coating. In preferred embodiments wherein the construct comprises a second linker, the second linker is attached to the coating and the first linker is attached to the second linker as described.

Preferably, the construct of this aspect comprises a plurality of glycopeptide antibiotic molecules.

Preferably, the construct of this aspect has an intermediate or high local density of glycopeptide antibiotic molecules.

In a second aspect there is provided a method of producing a construct, the method including the steps of obtaining (i) an optionally derivatized glycopeptide antibiotic; (ii) a nanoparticle; and (iii) a first linker, and connecting (i) and (ii) using (iii).

In a preferred embodiment of the second aspect, the method includes the step of:

(a) obtaining (i) an optionally derivatized glycopeptide antibiotic; (ii) a nanoparticle; (iii) a first linker; and (iv) a second linker;

(b) connecting (i) to (iii);

(c) connecting (ii) to (iv); and

(d) connecting (iii) to (iv).

Preferably the construct produced according to the method of this aspect is the construct of the first aspect.

In a third aspect there is provided a method of binding a construct to a microorganism or component thereof, the construct comprising (i) an optionally derivatized glycopeptide antibiotic; (ii) a nanoparticle; and (iii) a first linker connecting (i) and (ii), the method including the steps of:

(a) combining the construct and the microorganism or component thereof; and

(b) selectively binding the glycopeptide antibiotic of the construct with the microorganism or component thereof,

to thereby bind the construct to the microorganism or component thereof.

Preferably, the construct is the construct of the first or second aspect.

In a fourth aspect there is provided a method of separating a microorganism or component thereof from a sample using a construct, the method including the steps of:

(a) combining the construct with a sample containing the microorganism or component thereof, the construct comprising (i) an optionally derivatized glycopeptide antibiotic; (ii) a nanoparticle; and (iii) a first linker connecting (i) and (ii);

(b) selectively binding the microorganism or component thereof to the glycopeptide antibiotic of the construct; and (c) selectively obtaining the construct bound to the microorganism or component thereof from the sample using the nanoparticle,

to thereby separate the microorganism or component thereof from the sample using the construct.

Preferably the sample of step (a) is a sample obtained from a biological subject. Preferably the subject is a human or an animal. In some embodiments, the sample of step (a) is selected from the group consisting of urine, blood or a blood product including platelets, plasma, and serum.

In preferred embodiments the sample of step (a) is a blood sample. Preferably the blood sample comprises aggregated red blood cells, or is a sample obtained by red blood cell aggregation. Preferably, the blood sample is human blood.

Preferably, the construct of step (a) is the construct of the first or second aspects.

In preferred embodiments the microorganism of step (a) is a Gram positive bacteria. In some preferred embodiments the microorganism is a pathogenic microorganism.

Preferably, step (c) is performed using magnetic capture of a separation nanoparticle that is a magnetic nanoparticle.

In a fifth aspect, there is provided a method of analysing a microorganism or component thereof obtained from a sample, the method including the steps of:

(a) combining a construct with a sample containing a microorganism or component thereof, the construct comprising (i) an optionally derivatized glycopeptide antibiotic; (ii) a nanoparticle; and (iii) a first linker connecting (i) and (ii);

(b) selectively binding the microorganism or component thereof to the glycopeptide antibiotic of the construct; and

(c) selectively obtaining the construct bound to the microorganism or component thereof from the sample using the nanoparticle to thereby obtain the microorganism or component thereof from the sample; and

(d) analysing the microorganism or component thereof.

In preferred embodiments, steps (a)-(c) are as set forth for the fourth aspect.

According to the method of this aspect, analysis of the microorganism or component preferably includes identification of the microorganism.

The analysis according to the method of this aspect may be by any suitable strategy. In some preferred embodiments, the analysis is selected from the group consisting of matrix-assisted laser desorption/ionization (MALDI) mass spectrometry analysis; a fluorescence-based analysis; and a nucleic acid analysis. In one embodiment, the analysis includes determining the resistance profile of the microorganism.

In some preferred embodiments, the microorganism is removed from the construct prior to analysis.

In a sixth aspect there is provided a method of screening a sample for the presence of a microorganism or component thereof of interest, the method including the steps of:

(a) combining a construct with a sample, the construct comprising (i) an optionally derivatized glycopeptide antibiotic; (ii) a nanoparticle; and (iii) a first linker connecting (i) and (ii); and,

(b) selectively obtaining the construct from the sample using the nanoparticle; and

(c) performing analysis to determine if a microorganism or component thereof of interest is or was bound to the construct,

wherein a determination that the microorganism or component thereof of interest is or was bound to the construct indicates that the sample contains the microorganism or component thereof of interest, and a determination that the microorganism or component thereof of interest is or was not bound to the construct indicates that the sample does not contain the microorganism or component thereof of interest.

Preferably, the construct of step (a) is the construct of the first or second aspects.

In one preferred embodiment, the sample according to this aspect is a blood product, such as plasma or platelets. In another preferred embodiment, the sample according to this aspect is a urine sample.

In a seventh aspect there is provided a method of diagnosing a disease, disorder or condition in a biological subject, the method including the steps of identifying a microorganism in a biological sample obtained from a subject according to the method of the fifth aspect, and diagnosing a disease or condition in the subject based on the identity of the microorganism.

Preferably the disease or condition is caused by a Gram positive bacteria. In one preferred embodiment the disease or condition is a Gram positive bacterial infection. In particularly preferred embodiments the disease or condition is selected from sepsis or a urinary tract infection.

In an eighth aspect there is provided a method of treating a disease, disorder or condition, the method including the steps of diagnosing a disease, disorder or condition according to the method of the seventh aspect, and treating the disease or condition based on the diagnosis.

In a ninth aspect there is provided a method of inhibiting, controlling, or killing a microorganism, the method including the step of contacting a construct of the first aspect with a microorganism, to thereby inhibit, control, or kill the microorganism.

Preferably, the step of contacting the construct with the microorganism includes the step of selectively binding the glycopeptide antibiotic of the construct to the microorganism.

Preferably the microorganism is a Gram positive bacteria.

In certain embodiments the Gram positive bacteria is an antibiotic resistant bacteria. In certain embodiments, the microorganism shows at least partial resistance to the glycopeptide antibiotic of the construct when present as a free antibiotic.

In a tenth aspect there is provided a composition for treating or preventing a disease, disorder, or condition in a subject, the composition comprising a construct of the first aspect.

In an eleventh aspect there is provided a method of treating or preventing a disease, disorder, or condition in a subject, the method including the step of administering to the subject an effective amount of the construct of the first aspect or a composition of the tenth aspect, to thereby treat or prevent the disease, disorder, or condition in the subject.

In a twelfth aspect the invention provides for use of a construct of the first aspect in the manufacture of a composition for the treatment or prevention of a disease, disorder, or condition in a subject.

Preferably, the disease, disorder, or condition according to the tenth to twelfth aspects is a disease caused by a Gram positive bacteria. In some particularly preferred embodiments, the disease is bacterial sepsis. In another particularly preferred embodiment, the disease is a urinary tract infection.

In a thirteenth aspect, the invention is directed to a method of increasing the activity or efficacy of a glycopeptide antibiotic, the method including the step of connecting a glycopeptide antibiotic to a nanoparticle by a first linker. In preferred embodiments, the glycopeptide antibiotic, the nanoparticle, and the first linker are as set forth for the first aspect.

Preferably, the method includes the step of connecting a plurality of glycopeptide antibiotic molecules to the nanoparticle by a plurality of linkers. In these embodiments, preferably, the glycopeptide antibiotic is connected to the nanoparticle at an intermediate or high local density.

In a first aspect of a second broad form, the present invention is directed to a compound comprising an optionally derivatized glycopeptide antibiotic bound to a first linker. Compounds of this broad form will be suitable for connection to a nanoparticle, to form constructs of the first aspect of the first broad form.

Preferably, the glycopeptide antibiotic of the second broad form is selected from the group consisting of vancomycin; teicoplanin; oritavancin; telavancin; chloroeremomycin; and balhimycin. Preferably the glycopeptide antibiotic is vancomycin.

In some preferred embodiments, the first linker of the second broad form comprises a PEG group, preferably at least PEGS.

In some preferred embodiments, the first linker comprises a linear carbon chain, wherein the linear carbon chain is C4-C12. Preferably the linear carbon chain is C8.

In preferred embodiments, the first linker comprises an amine-derived moiety. Preferably the amine-derived moiety is at a first end of the first linker. Preferably the amine-derived moiety connects the linker to the glycopeptide antibiotic. The connection may be an amide bond.

In one preferred embodiment, the first linker comprises an azide or azide-derived azide-derived moiety. Preferably, the azide or azide-derived moiety is at a second end of the linker opposite the glycopeptide antibiotic.

In some embodiments, the compound comprises a second linker. Preferably, the second linker is as described for the first aspect.

In a second aspect of the second broad form, there is provided a dimer compound comprising a compound of the first aspect of the second broad form, wherein the glycopeptide antibiotic is vancomycin, connected to a further vancomycin moiety via the first linker.

In preferred embodiments, the dimer compound of this aspect of the second broad form comprises two connected compounds of the first aspect of the second broad form, wherein the glycopeptide antibiotics of the respective compounds are vancomycin. In certain embodiments, the two connected compounds are connected via a moiety comprising a linear carbon chain and/or PEG moiety.

In some embodiments, the compound of the first or second aspects of the second broad form has increased activity or efficacy relative to the glycopeptide antibiotic.

A further aspect of the second broad form provides a method of increasing the activity or efficacy of an glycopeptide antibiotic, the method including the step of connection the glycopeptide antibiotic to a first linker. A related aspect provides a method of increasing the activity or efficacy of vancomycin, including the step of connecting one vancomycin and another vancomycin by a first linker.

It will be appreciated that the indefinite articles “a” and “an” are not to be read herein as singular indefinite articles or as otherwise excluding more than one or more than a single subject to which the indefinite article refers. For example, “an” antibiotic includes one antibiotic, one or more antibiotics, or a plurality of antibiotics.

As used herein, unless the context requires otherwise, the words “comprise”, “comprises” and “comprising” will be understood to mean the inclusion of a stated integer or group of integers but not the exclusion of any other integer or group of integers.

### BRIEF DESCRIPTION OF THE FIGURES

In order that the invention may be readily understood and put into practical effect, preferred embodiments will now be described by way of example with reference to the accompanying figures.

FIG. 1 sets forth a schematic depiction of synthesis of a preferred construct according to the invention.

FIG. 2 sets forth potential sites to facilitate addition of a first linker to vancomycin.

FIG. 3 sets forth NMR characterization of N₃-PEG3-Van (labelled ‘N₃-Vancomycin’). ¹H NMR (600 MHz, DMSO-d₆) and ¹³C NMR (125 MHz, DMSO-d₆).¹ Abbreviations br=broad; d=doublet; m=multiplet; non=nonet; o=obscured; quin=quintet; s=singlet; t=triplet; v br=very broad; q=quartet.

FIG. 4 sets forth LC and -MS of N₃-PEG3-Van.

FIG. 5 sets forth the fraction of aggregated and dispersed constructs (NPs) as determined by dynamic light scattering.

FIG. 6 sets forth A) Ac-Kaa structure. B) N₃-NBD structure. C) Fam-Kaa structure.

FIG. 7 sets forth A) quantification standard curves for Fam-Kaa. B) Quantification standard curve for LCMS of eluted Ac-Kaa. C) LC and -MS of eluted Ac-Kaa.

FIG. 8 sets forth A) Quantification of DBCO layer formation as measured by the fluorescence signal of N₃-NBD dye. B) Comparison between the quantification of conjugated N₃-PEG3-Van based on binding of Fam-Kaa, calculated reacted cyclooctyne detected by N₃-NBD fluorescence, or binding then elution of Ac-Kaa measured by LCMS.

FIG. 9 sets forth a schematic representation of a process for bacterial bioseparation using a construct of the invention. Step 1—Blood collection and subsequent RBC aggregation. Step 2—Bacterial magnetic capture using Gram positive-specific bioseparation constructs of the invention, with subsequent washing and removal of interfering cells. For certain applications, the process can additionally include: Step 3—Bacterial elution; Step 4—Complete bacterial lysis (and optionally DNA capture using silica nanoparticles); and/or Step 5—DNA purification, concentration and elution.

FIG. 10 sets forth bacterial capture efficiency from platelets of S. aureus strain (ATCC 25923) and S. epidermidis strain (ATCC 12228) as assessed by culture. Spiked bacterial cells were captured from platelets without any prior treatments. Capture efficiency was extremely high, with essentially all bacteria spiked into the samples captured. The more than 100% capture efficiency calculated was due to bacterial replication during the capturing step which allowed the new daughter bacterial cells to be captured with the bioseparation constructs of the invention. Data represent mean (n=3)±SD.

FIG. 11 sets forth capture efficiency in blood solution with sensitive and resistant Gram-positive strains. Capture efficiency was high for all Gram-positive strains assessed. The more than 100% capture efficiency calculated for some strains was due to bacterial replication during the capturing step which allowed the new daughter bacterial cells to be captured with the bio separation constructs of the invention. Capture efficiency of S. epidermidis strains was somewhat lower than other strains with an efficiency of 93% and 77% for sensitive and resistant strain, respectively, with high local density constructs. For the other strains, data is for low local density constructs. All data represent mean (n=3)±SD.

FIG. 12 sets forth capture of Gram negative E. coli using bioseparation constructs. The specificity of the bioseparation constructs was high, with only ˜3-4% capture of E. coli. All data represent mean (n=3)±SD

FIG. 13 sets forth A) Whole blood induced RBCs aggregation analysis as measured by hemocytometer and bacterial culture. Square: Effect of RBCs aggregation on bacterial count in spiked whole blood sample by time (n=3). Circle: Reduction of total RBCs and WBCs count in whole blood sample measured using hemocytometer by time (n=4). Data are shown as means±SD, some error bars are too small to be visible in the graph. B) DNA purity obtained after bioseparation of bacteria and subsequent bacterial lysis, DNA capture, and DNA purification. Nanodrop measurement of DNA obtained from a human blood sample spiked with 10³ (cfu/mL) S. aureus.

FIG. 14 sets forth A) qPCR CT values of 1 μL of 10⁹ cfu/mL S. pneumoniae extracted DNA by Qiagen kit (Square) and bioseparation and subsequent DNA extraction as described in Example 2 using a construct of the invention, which is referred to as ‘Bac-ID’ (Circle). B) Comparison of the human DNA content in whole blood using Qiagen DNeasy Blood and Tissue commercial kit (by nanodrop) and the Bac-ID method (by qPCR) (n=4). C) Comparison of Qiagen DNeasy Blood and Tissue kit extraction efficiency in blood and detection limit in PBS buffer (Final elution volume of DNeasy Blood and Tissue kit and of the Bac-ID sample is 200 and 20 μL, respectively) (n=3). Sample of 10⁹ S. aureus (cfu/mL) was only assessed by Qiagen kit to assess inhibition of the kit with blood samples. Samples with high concentration (10⁹ S. aureus cfu/mL) were not assessed by the Bac-ID method, as the method was developed to ensure high sensitivity with low bacterial counts. Data are shown as means±SD; some error bars are too small to be visible in the graph.

FIG. 15 sets forth A) qPCR analysis of Bac-ID extracted-bacterial DNA (left Y-axis) and human DNA content (right Y-axis) at 10⁷, 10⁵, 10³ (cfu/mL) and no bacteria (n=3). B) Sensitive and resistant gram-positive qPCR detection of bacterial DNA extracted from spiked blood using Bac-ID (n=3). C) S. aureus qPCR limit of detection CT values versus number of bacterial cells by culture extracted from spiked blood (n=6-8). D) qPCR CT values of six S. aureus replicate samples of ≤5 cfu/mL (n=8). Data are shown as means±SD; some error bars are too small to be visible in the graph.

FIG. 16 sets forth qPCR analysis of Bac-ID extracted-bacterial DNA from 1 and 10 mL blood. 10² cfu S. aureus was spiked and extracted from 1 mL (circle) and 10 mL (triangle), in parallel to extraction of 10² (cfu/mL) from 10 mL samples (square) (n=4). Stars in graph represent means (n=4). Error bars shown are means±SD.

FIG. 17 sets forth human DNA content comparison at increased incubation time from 1 and 10 mL blood samples extraction using Bac-ID.

FIG. 18 Bacterial membrane damage hypothesis showing the binding ratio of Lipid II terminal D-alanyl-D-alanine residues to one molecule of: A) Unconjugated vancomycin, B) low density Van-NPs and C) high density Van-NPs. The figure illustrates the reason behind the difference in binding potency of 1 molecule of unconjugated vancomycin compared to Van-NPs at different local densities.

FIG. 19 sets forth estimated values of conjugated vancomycin in low, medium, and high local density constructs of the invention, based on fluorescence of Fam-Kaa bound to coupled N₃-PEG3-Van, fluorescence of N₃-NBD bound to unreacted DBCO, or concentration of Ac-Kaa bound to conjugated N₃-PEG3-Van then eluted and measured by LCMS.

FIG. 20 sets forth comparison of MIC values for free vancomycin, N₃-PEG3-Van (labelled ‘N₃-Vancomycin’), and constructs (labelled ‘Van-NPs’) as described in Example 1, against vancomycin sensitive and resistant strains (n=3). The MIC value for constructs is based on the calculated conjugated vancomycin content, taking into account vancomycin density and number of nanoparticles.

FIG. 21 sets forth the relationship between the NP concentration required to achieve inhibition against sensitive and resistant S. aureus with four different Van-NPs loading densities.

FIG. 22 sets forth the relationship between MIC values against sensitive and resistant S. aureus for constructs with different loading densities of vancomycin.

FIG. 23 sets forth binding affinity of free vancomycin; N₃-PEG3-Van (labelled ‘N₃-Vancomycin’); and constructs (labelled ‘Van-NPs’) as described in Example 1 with low, intermediate, and high-density vancomycin with a bacterial ligand, as measured by prevention of inhibitory activity in the presence of added synthetic Ac-Kaa (n=3).

FIG. 24 sets forth membrane permeability of propidium iodide to bacteria in the presence of vancomycin and constructs (labelled ‘Van-NPs’) with different loading densities (n=3).

FIG. 25 sets forth DiSC₃(5) membrane permeabilization fluorescence values of different local densities of Van-NPs, vancomycin and N₃-vancomycin after incubation for 30 min (A) and 90 min (B). Van-NPs and vancomycin were used at MIC concentration. Controls of HSA-NPs, 0.1% Triton-X and sterile water were incubated at the same conditions. (C) Normalized fluorescence values of DiSC₃(5) dye assessing membrane permeability in the presence of vancomycin, N₃-vancomycin and Van-NPs with different local densities (at 1× MIC). Data (n=2) are shown as means±SD, some error bars are too small to be visible in the graph.

FIG. 26 sets forth Transmission electron microscopy imaging (JEOL 1011) of ruptured bacterial membrane after treatment with high density Van-NPs (0.05 μg/mL, below MIC) (arrows) (Scale bar is 1 μm).

FIG. 27 sets forth total cellular ATP leakage concentration in the presence of vancomycin and Van-NPs with different densities (1× MIC). All data (n=2) are shown as means±SD, some error bars are too small to be visible in the graphs.

FIG. 28 sets forth the structure of certain preferred compounds comprising a glycopeptide antibiotic and a first linker according to embodiments of the invention. A) N₃-C8-Van B) N₃-PEG3-Van.

FIG. 29 sets forth an illustrative diagram of Gram-positive fluorescence detection using two sets of a magnetic nanoparticles of the invention, and a separate fluorescent nanoparticle.

FIG. 30 sets forth fluorescent detection of different concentrations of S. aureus strain from blood samples (n=3).

FIG. 31 sets forth a vancomycin-linker-vancomycin dimer compound of the invention.

FIG. 32 sets forth a schematic illustration of synthesis of a vancomycin-linker-vancomycin dimer compound of the invention.

FIG. 33 sets forth a comparison of MIC values for various Gram positive bacterial strains, for N₃-PEG3-Van dimer (21a; labelled as ‘Vanco-PEG-3-Tz-6C dimer’) and N₃-C8-Van dimer (21b; labelled as ‘Vanco-8C-Tz-6C’ dimer), and in comparison to free vancomycin.

FIG. 34 sets forth a schematic of the conjugation process described in PCT/AU2015/050564.

FIG. 35 sets forth a schematic of reaction of PDEA dextran polymer with magnetic nanoparticles and nano-gold to form a polymer multilayer.

FIG. 36 sets forth estimation of vancomycin surface concentration using Fam-Kaa binding on nanoparticles for which conjugation using PDEA dextran polymer has been performed. (A) Linear scale. (B) Log scale.

FIG. 37 sets forth capture of Staphylococcus aureus (ATCC 25923) using vancomycin nanoparticles A) functionalized using PDEA dextran polymer; and B) functionalized using HSA. MNPs functionalized using both of these approaches were successful in selective enrichment of S. aureus.

FIG. 38 sets forth Fam-Kaa fluorescence as a measure of surface concentration of vancomycin for constructs with first linkers comprising PEG3, C3, or C8, respectively.

FIG. 39 sets forth Gram positive bacterial capture using constructs with first linkers comprising PEG3 (A), C8 (B), or C3 (C), respectively.

FIG. 40 sets forth Minimum Inhibitory Concentration (MIC) of vancomycin and vancomycin adducts N3-PEG3-Van, N3-C3-Van, and N3-C8-Van for S. aureus, S, pneumonia, E. faecium, and E. faecalis.

FIG. 41 sets forth a schematic depiction of N3-PEG3-Van conjugated to magnetic nanoparticles using a PDEA dextran polymer approach.

FIG. 42 sets forth an estimation of thiols on the surface of magnetic nanoparticles by Ellman's reaction. MNPs were successfully thiolated using Cystamine Hydrochloride for subsequent addition of PDEA polymer to build a monolayer of polymer on the nanoparticle surface.

FIG. 43 sets forth a qualitative estimation of gold incorporation during multilayer polymer formation using absorbance spectra of nano-gold. Nanogold used for multilayer generation has an absorption max at 520 nm. Decrease on the OD of the supernatant shows that nanogold was incorporated on the layers of polymer, hence indirectly assuring the formation of multilayer of polymer on the surface of magnetic nanoparticles for efficient blocking and subsequent conjugations.

### DETAILED DESCRIPTION

This invention relates to the design and production of constructs comprising glycopeptide antibiotics and nanoparticles. Such constructs include constructs for bioseparation, and constructs with antimicrobial activity. The invention also relates to compounds or ‘adducts’ comprising an optionally derivatized glycopeptide antibiotic bound to a first linker and/or second linker, suitable for connection to nanoparticles to form such constructs.

The invention is at least partly predicated on the recognition that such constructs may offer important advantages for separation and/or detection of microorganisms in biological samples. Furthermore, the invention is at least partly predicated on the surprising discovery that such constructs comprising a glycopeptide antibiotic and a nanoparticle may have substantially increased activity or efficacy as compared to the corresponding antibiotic itself.

The invention is also at least partly predicated on the discovery of design parameters with surprising benefits for binding of Gram positive bacteria using constructs as herein described. In particular, it has been surprisingly found that the use of certain linkers to connect glycopeptide antibiotics to nanoparticles has particular advantages for binding efficiency of such constructs against Gram positive bacteria. Such advantages for binding efficiency may lead to advantages for separation of Gram positive bacteria from samples using constructs of the invention, and/or for antimicrobial activity of constructs of the invention against Gram positive bacteria.

### Constructs

Constructs of the invention will comprise an optionally derivatized glycopeptide antibiotic connected to a nanoparticle. As used herein, unless the context requires otherwise, the terms “connect”, “connection”, “connected” etc., will be understood to encompass direct connection (e.g. direct binding), or indirect connection (e.g. connection via one or more other molecules or moieties).

One aspect of the invention relates to a construct comprising: (i) an optionally derivatized glycopeptide antibiotic; (ii) a nanoparticle; and (iii) a first linker connecting (i) and (ii).

As used herein, a “derivatized” glycopeptide antibiotic broadly encompasses glycopeptide antibiotics comprising one or more modifications or alterations such as transformations of existing functional groups and introduction of temporary protecting groups and the like. The term is also considered to include all salt forms. Preferably, the derivatized glycopeptide antibiotic will be a biologically active derivative, which retains at least a part of one or more biological activities of a corresponding non-derivatized or unmodified glycopeptide antibiotic. Biological activities of a glycopeptide antibiotic may include Gram positive bacteria binding, and/or antimicrobial activity, although without limitation thereto.

In some preferred embodiments the derivatized glycopeptide antibiotic retains at least: 10%; 20%; 30%; 40%; 50%; 60; 70%; 80%; or 90% of one or more biological activities. For an example of modifications of glycopeptide antibiotics that may result in biologically active derivatives, the skilled person is directed to Malabarba et al. (1997) Medicinal Research Reviews, 17(1) 69-137, incorporated herein by reference.

The construct of this aspect may have the following general structure:

A-L₁-N
wherein:
A is the optionally derivatized glycopeptide antibiotic;
L₁ is the first linker; and
N is the nanoparticle.

It will be appreciated that the respective connections between A and L₁; and L₁ and N, may be direct or indirect connections, as hereinabove described. In a preferred embodiment, the connection between A and L₁ is a direct connection. In a preferred embodiment, the connection between L₁ and N is an indirect connection.

In a preferred embodiment of this aspect, the construct comprises:
(i) an optionally derivatized glycopeptide antibiotic;
(ii) a nanoparticle;
(iii) a first linker connected to (i); and
(iv) a second linker connected to (ii),

wherein (iii) is connected to (iv) and (i) is connected to (ii).

The construct of this embodiment may have the following general structure:

A-L₁-L₂-N
wherein:
A is the optionally derivatized glycopeptide antibiotic;
L₁ is the first linker;
L₂ is the second linker; and
N is the nanoparticle.

It will be appreciated that the respective connections between A and L₁; L₁ and L₂; and L₂ and N may be direct or indirect connections, as hereinabove described. In preferred embodiments, the connection between A and L₁ is a direct connection. In preferred embodiments, the connection between and L₁ and L₂ is a direct connection. In preferred embodiments, the connection between L₂ and N is an indirect connection.

In embodiments of the invention wherein the nanoparticle of the construct is passivated with a coating, the construct of the first aspect may be depicted as follows:

A-V-L₁-W-L₂-X—P—Z—N

wherein:

A is the optionally derivatized glycopeptide antibiotic;

V is a functionality of the first linker (L₁) linking L₁ to A;

W is a functionality of L₁ linking L₁ to the second linker (L₂);

X is a functionality of L₂ linking L₂to P;

P is a passivation coating;

Z is a functionality linking P to N; and

N is the nanoparticle.

Preferably:

V comprises an amide bond;

W comprises a triazole;

X comprises an amide or disulphide bond;

P comprises human serum albumin (HSA) or a polymer coating; and

Z comprises an amide and/or a disulphide bond.

Alternatively, in certain embodiments, constructs of the invention may be depicted as follows:

A-V—R₁—W—R₂—X—N

wherein:

A is the optionally derivatized glycopeptide antibiotic;

V—R₁—W is the first linker comprising functional groups V and W on first and second ends, respectively, and an internal moiety R₁;

R₂—X is the second linker comprising moiety R₂ and functional group X; and

N is the nanoparticle.

Preferably:

V comprises an amide bond connecting the first linker to A;

R₁ comprises a PEGN moiety (as hereinbelow described) and/or a linear carbon chain;

W comprises a triazole connecting the first linker and R₂ of the second linker;

R₂ comprises a PEGN moiety and/or a linear carbon chain;

X comprises an amide and/or disulphide bond connecting the second linker to the nanoparticle.

The structure of particularly preferred constructs of the invention may be depicted as follows:

A-V-PEG3-W-DBCO-Y-PEG4-X—P—Z—N

wherein:

A, V, W, X, and N are as for the directly preceding embodiment;

V-PEG3-W is the first linker, comprising V (amide bond) and W (triazole) on first and second ends, respectively; and an internal PEG3 moiety;

DBCO-Y-PEG4-X is the second linker, comprising a dibenzocyclooctyl (DBCO)-derivative (henceforth referred to as DBCO in this context) and functional group X (amide bond) on first and second ends, respectively, an internal PEG4 moiety, and moiety Y located between DBCO and PEG4; and

P—Z—N is a passivated nanoparticle (N), wherein P is a passivation coating and Z is a functional group connecting P to N.

Preferably:

Amide bond X directly connects the second linker to the passivation coating of the passivated nanoparticle;

Y comprises an amide bond connecting DBCO and PEG4;

P is human serum albumin (HSA); and

Z comprises an amide bond directly connecting N to P.

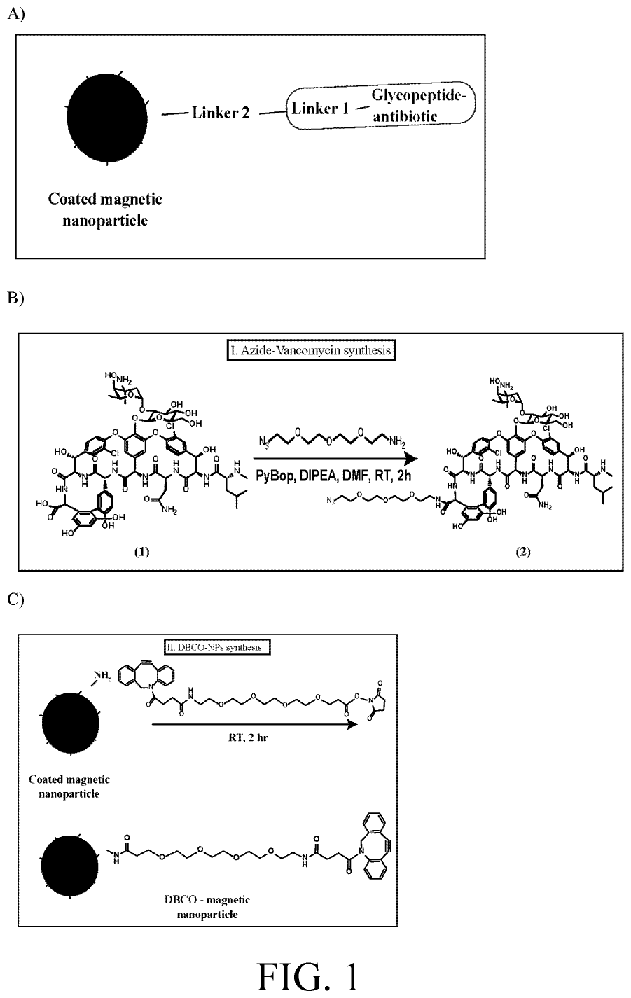

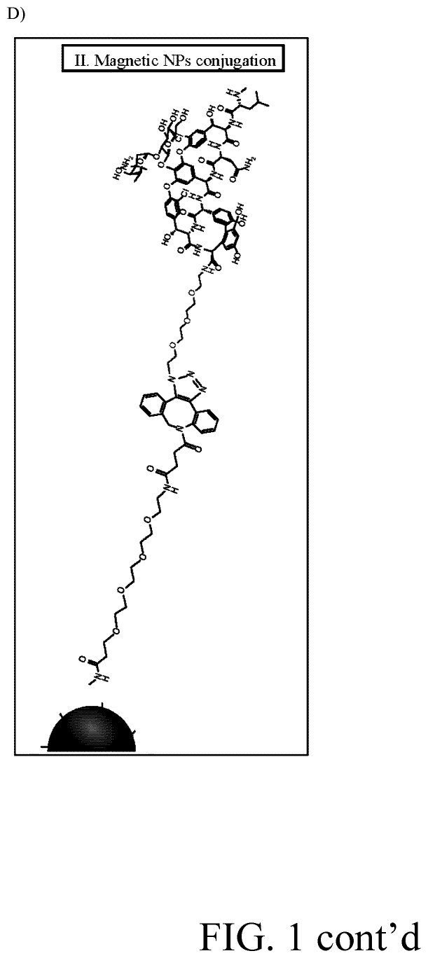

An exemplary embodiment of a construct in the form A-V-PEG3-W-DBCO-Y-PEG4-X—P—Z—N as set forth above is provided in FIG. 1D.

In another alternative construct falling within the scope of this aspect, the first linker may be directly connected to a passivated nanoparticle and the glycopeptide antibiotic. In a preferred embodiment of this alternative construct, the first linker is bound to the passivated nanoparticle via a disulphide group. In some embodiments, the first linker is bound to the passivated nanoparticle via a thioether bond. In some such embodiments the thioether bond may be formed by reaction of a thiol group of the polymer coating with a maleimide moiety from the second end of a precursor to the first linker. It will be appreciated that preferred embodiments of this alternative construct need not necessarily comprise a second linker.

Some such alternative constructs may have a general structure of:

A-V-L₁-U—P—Z—N

wherein:
A is the optionally derivatized glycopeptide antibiotic;
V is a functionality of the first linker (L₁) linking L₁ to A;
P is a polymer coating;
N is the nanoparticle;
U comprises a disulphide and/or thioether bond connecting L₁ to P;
Z comprises an amide and/or disulphide bond connecting N to P;

Certain embodiments falling within the scope of this construct include:
A-CONH-L₁-U—P—SS—CH₂CH₂—NHCO—N;
A-CONH-L₁-U—P—SS-PEGS-NHCO—N; and
A-CONH-L₁-U—P—SS—N

It will be appreciated that functional groups (e.g. V, W, and X set forth above) joining individual components (e.g. L₁; L₂; A; and N set forth above) of constructs of this aspect may be described herein as functional groups of or belonging to a particular component of the construct. It will be understood that this designation is not limiting with respect to precursor components that may be used to form the construct, such as in preferred embodiments as herein described. That is, description of a functional group as belonging to a given component does not imply any specific relationship of the functional group with a precursor of that component.

### Bioseparation

Some preferred constructs of the invention are optimized for separation of microorganisms, such as Gram positive bacteria, from samples, and may be referred to herein as “bioseparation constructs”. In some preferred embodiments, the bioseparation constructs are optimized for separation of Gram positive bacterial pathogens from human blood samples, which will be understood to include blood products such as platelets and plasma samples. In some preferred embodiments, the bioseparation constructs are optimized for separation of Gram positive bacterial pathogens from urine samples.

It will be appreciated however, that such bioseparation constructs of the invention may have application for separation of both pathogenic and non-pathogenic Gram positive bacteria from any suitable sample. Such samples may include laboratory samples, such as artificial cultures; and environmental samples such as soil and water samples. Such samples may also include any suitable biological sample, including samples from plants and animals.

In particular regard to animal samples, in addition to human samples, the sample may be from another primate (e.g. apes and monkeys); a canine; a feline; an ungulate (e.g. equine, bovine, and swine); or an avian, although without limitation thereto. The animal may be livestock (e.g. horses, cattle and sheep), a companion animals (e.g. dogs and cats), a laboratory animals (e.g. mice, rats and guinea pigs) or a performance animal (e.g. racehorses, greyhounds and camels), although without limitation thereto. The animal sample may be of a mammalian or non-mammalian species.

It will be further understood that, in addition to blood and urine samples, the animal sample may be any suitable sample. For example, the sample may be an animal tissue sample including a muscle; epithelial; connective; or nervous tissue sample.

Additionally, although bioseparation constructs as herein described are primarily intended for separation of Gram positive bacteria, it will be appreciated that separation of other microorganisms which bind to glycopeptide antibiotics is also within the scope of the invention. By way of non-limiting example, it will be appreciated that certain glycopeptide antibiotics have been shown to inhibit fungal growth, although the mechanism of inhibition (and in particular, whether this involves binding to fungal components) is presently unclear in at least many instances.

As hereinbelow described, preferred constructs of this aspect bind Lipid II and/or peptidoglycan, wherein the optionally derivatized glycopeptide antibiotic binds to the Lipid II and/or peptidoglycan. Although such constructs of this aspect that bind Lipid II and/or peptidoglycan will typically be suitable for bio separation of Gram positive microorganisms as set forth above, it will be readily appreciated that such constructs will also typically be suited to bio separation of Lipid II and/or peptidoglycan itself, regardless of origin, provided that the optionally derivatized glycopeptide antibiotic of the construct can access and bind the Lipid II and/or peptidoglycan. It will be appreciated that the Lipid II and/or peptidoglycan may be bioseparated after being obtained or produced by any suitable means, such as extraction from a microorganism, or by synthetic production.

### Antimicrobial Activity

Some preferred constructs of the invention are optimized for increased or enhanced antimicrobial activity or efficacy, as compared to the glycopeptide of the construct alone. Such constructs may be referred to herein as “antimicrobial constructs”. Preferred such antimicrobial constructs are optimized for inhibition, control, or killing of Gram positive bacteria. However, it will be appreciated that increased or enhanced activity against any other microorganism (such as certain fungi) which is affected by glycopeptide antibiotics is also within the scope of the invention.

It will be understood that some constructs of the invention may be both bioseparation constructs and antimicrobial constructs.

### Glycopeptide Antibiotics

As used herein a “glycopeptide antibiotic” will be understood to be a glycosylated peptide with at least some capacity to (i) bind and/or (ii) inhibit growth, proliferation or viability of a microorganism. As used herein, the ability to inhibit growth, proliferation and/or viability of a microorganism may be referred to generally as “antimicrobial” activity.

Typically, glycopeptide antibiotics comprise cyclic or polycyclic peptides. Glycopeptide antibiotics typically have properties consistent with microbial, nonribosomal origin. However, it will be appreciated that all suitable compounds regardless of origin, e.g. both isolated natural compounds and synthetically produced or recombinant compounds, are encompassed by the term glycopeptide antibiotic as used herein. For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state.

Glycopeptide antibiotics of constructs of the invention preferably have capacity to bind Gram positive bacteria. It will be appreciated by the skilled person that glycopeptide antibiotics typically bind to peptidoglycan in the bacterial cell wall and to the Lipid II precursor of peptidoglycan. Typically, glycopeptide antibiotics bind selectively to Gram positive bacteria, with limited or absent binding activity towards Gram negative bacteria.

The glycopeptide antibiotic of the construct may be any suitable glycopeptide antibiotic. In embodiments, the glycopeptide antibiotic is selected from the group consisting: of vancomycin; teicoplanin; oritavancin; telavancin; dalbavancin; chloroeremomycin; and balhimycin. In one particularly preferred embodiment, the glycopeptide antibiotic is vancomycin.

It will be appreciated that, typically, binding of peptidoglycan of the cell wall of Gram positive bacteria by glycopeptide antibiotics occurs in a substantially non-species or strain-specific manner. That is, glycopeptide antibiotics will generally have broad binding activity for Gram positive bacteria as a group. It will be further understood that glycopeptide antibiotics will generally bind to Gram positive bacteria regardless of whether the bacteria is sensitive or resistant to the antibiotic.

As hereinbelow described, in certain embodiments, constructs of the invention (sometimes referred to as bioseparation constructs) are designed to obtain Gram positive bacteria from a sample, wherein the glycopeptide antibiotic binds the Gram positive bacteria to the construct. It will be appreciated that in such embodiments, binding of the glycopeptide antibiotic may or may not inhibit growth, proliferation, or viability of the bound Gram positive bacteria.

As hereinbelow described, microorganisms obtained using bioseparation constructs of the invention may be used for any suitable downstream application. It will be appreciated that for some downstream applications (e.g. nucleotide sequencing), the viability of a microorganism obtained using the bioseparation construct may be of limited or no significance for the downstream application. For other downstream applications, viable or non-viable microorganisms may be preferred. Accordingly, for these embodiments, a suitable antibiotic that is optimized to either maintain or disrupt viability of a microorganism of interest may be selected.

It will be further appreciated that constructs according to certain embodiments of the invention (sometimes referred to as antimicrobial constructs) are designed to inhibit or kill microorganisms, typically Gram positive bacteria, to which the glycopeptide antibiotic of the construct binds. Accordingly, for these embodiments, a suitable antibiotic that is optimized to have at least partial antimicrobial activity against a microorganism of interest may be selected.

### Nanoparticles

Nanoparticles of constructs of the invention may take a range of suitable forms.

In embodiments wherein the construct is a bioseparation construct, the nanoparticle will be designed to facilitate separation of the construct from a sample. Such nanoparticles may be referred to as ‘separation nanoparticles’. As used herein, the term “separation nanoparticle” will be understood to refer to any nano-scale particle with suitable properties to allow selective removal from a sample. Suitable properties of a separation nanoparticle will depend, at least in part, on the nature of the sample from which it is to be selectively removed. Suitably, the separation particle will have structural and/or chemical properties that are different from other components of the sample.

Preferably, the separation nanoparticle is a magnetic nanoparticle. The magnetic nanoparticle may take any suitable form, and will be understood to include nanoparticles containing magnetic components, such as ‘magnetic grains’. Such magnetic nanoparticles can be formed from a variety of suitable materials. Some preferred materials for use in magnetic nanoparticles include iron, nickel, and cobalt, although without limitation thereto. ‘Superparamagnetic’ nanoparticles are particularly preferred for the invention. Such nanoparticles comprise small, thermally agitated magnets in carrier liquids (known as ‘ferrofluids’). The skilled person is directed to Neuberger et al. (2005) Journal of Magnetism and Magnetic Materials. 293(1) 483-496 (incorporated herein by reference) for a review of superparamagnetic nanoparticles.

Typically, magnetic nanoparticles of the invention will range in diameter from about 1 nm to about 500 nm. In some preferred embodiments, magnetic nanoparticles of the construct have a diameter of about 50 nm to about 300 nm, including about: 75 nm; 100 nm; 125 nm; 150 nm; 175 nm; 200 nm; 225 nm; 250 nm; and 275 nm.

In embodiments of the invention wherein the construct is an antimicrobial construct, the nanoparticle may be a separation nanoparticle, such as a magnetic nanoparticle as described above.

In preferred embodiments wherein the construct is an antimicrobial construct, the nanoparticle is a nanoparticle suitable for therapeutic delivery. In these embodiments, the nanoparticle may be a biodegradable nanoparticle, such as a biodegradable nanoparticle formed from one or more of protein; polysaccharides; and synthetic biodegradable polymers. For an overview of nanoparticles for therapeutic delivery, the skilled person is referred to Nanoscale Materials in Targeted Drug Delivery, Theragnosis and Tissue Regeneration (Springer 2016, Sudesh Kumar Yadav Ed.), and ‘Biodegradable Nanoparticles and Their In Vivo Fate’ therein (which is incorporated herein by reference).

The surface of the nanoparticle of constructs of the invention will suitably comprise one or more functional groups allowing attachment of the nanoparticle to another component of the construct. In one particularly preferred embodiment, the functional group is a carboxyl group.

In some embodiments, the functional groups may facilitate direct attachment of the separation nanoparticle to the first linker. In other embodiments, the functional groups may facilitate attachment of the separation nanoparticle to the second linker.

In particularly preferred embodiments, the separation nanoparticle is passivated. In these embodiments, preferably, the functional group of the surface of the separation nanoparticle facilitates passivation of the nanoparticle (see, e.g., FIG. 1). It will be appreciated that, in preferred embodiments of the invention wherein the surface of the separation nanoparticle is passivated, passivation can reduce non-specific binding to the surface of the nanoparticle. In particular, passivation of the surface of the separation nanoparticle of constructs of the invention may reduce non-specific binding of non- Gram positive bacterial components. It will be further appreciated that the passivation coating may include multiple layers.

Passivation will typically be by way of coating the surface of the separation nanoparticle. Suitably, the coating is attached to the surface of the separation nanoparticle by reaction with a functional group on the surface of the separation nanoparticle. Suitably, functional groups on the coating facilitate attachment of the separation nanoparticle to the first or second linker. Suitable functional groups may include carboxylate groups, amine groups, thiol groups and maleimide groups.

In some embodiments, the coating is a protein coating. In one particularly preferred embodiment, the protein coating is human serum albumin (HSA). In this embodiment, preferably the HSA is attached to the nanoparticle via an amide moiety (see, e.g., FIG. 1).

In some embodiments, the coating is a polymer coating. In embodiments the polymer coating is or comprises carboxymethyl-PDEC-dextran (CMD). In embodiments, the polymer coating is or comprises PDEC-dextran. In embodiments, the polymer coating is or comprises 2-(pyridinyldithio)ethaneamine (PDEA)-dextran. In embodiments, the polymer coating may comprise nano-metal components, which may function in binding multiple polymer layers. The polymer coating may be attached to the nanoparticle via an amide and/or disulfide moiety.

A particularly preferred embodiment wherein the polymer coating contains multiple layers of PDEA-dextran, bound with nano-metal components in the form of nano-gold particles, is described in Example 7.

### First Linkers

First linkers of constructs of the invention facilitate connection between the glycopeptide antibiotic and the nanoparticle.

It has been surprisingly found for the invention that constructs comprising certain first linkers are advantageous for binding to Gram positive bacteria. Without wishing to be bound by theory, based on experimental observations it is considered that molecules which are at least partially hydrophilic may be particularly effective for capture using constructs of the invention. In particular, partially hydrophilic or hydrophilic linkers e.g. those comprising PEG, may provide an extended tether for the glycopeptide antibiotic, with limited or absent coiling occurring in an aqueous environment.

In particularly preferred embodiments wherein the first linker is at least partially hydrophilic, the linker comprises a polyethylene glycol (PEG) group. As will be readily understood by the skilled person, PEG molecules may be expressed, when located internally within a larger molecule as in constructs of the invention, in the form Rₐ—(O—CH₂—CH₂)n-, where ‘n’ is the number of PEG monomers and Rₐ is a carbon chain connecting the PEG. As used herein, PEG molecules may be expressed in the form ‘PEGN’, wherein ‘N’ is the number of PEG monomers.

It is particularly preferred that Rₐ comprises a C2-C4, or more preferably C2 linear carbon chain. Such PEG-containing moieties may take the form —(CH₂)ₘ(O—CH₂—CH₂)ₙ—, where ‘m’ is 2 to 4 carbons e.g. —CH₂—CH₂—(O—CH₂—CH₂)₃—. It will be appreciated that a determination of the particular side or end of the PEG moiety to which a linear carbon chain, such as a C2 chain, is adjacent, may be dependent upon the end of the combined PEG-linear carbon chain molecule that is considered to be the ‘start’, or ‘first end’ of the PEG-linear carbon chain molecule.

Preferably, the PEG group is at least PEG3. Preferably the PEG group is in the range PEG3 to PEG10, including PEG4; PEG5; PEG6; PEG7; PEG8; and PEG9. In some particularly preferred embodiments, the PEG group is PEG3 or PEG4.

It will be appreciated that partially hydrophilic first linkers may comprise other components. In some embodiments, the partially hydrophilic first linker may comprise a hydrophobic component. In some embodiments, the hydrophobic component may be a linear carbon chain. The linear carbon chain may be a C2-C15 chain, including C3; C4; C5; C6; C7; C8; C9; C10; C11; C12; C13; and C14, although without limitation thereto.

Certain hydrophobic linkers may also be suitable or desirable according to constructs of the invention. It has been recognised that, where hydrophobic linkers are used, molecules comprising at least a particular linear chain length may be more effective for capture using constructs of the invention.

In certain embodiments, the linker is a hydrophobic molecule comprising a linear carbon chain of at least four carbons. Preferably the linear carbon chain is C4-C20, including C5; C6; C7; C8; C9; C10; C11; C12; C13; C14; C15; C16; C17; C18; and C19, or preferably C6-C10.

In some embodiments, the linear carbon chain may be C6-C16; C6-C14; C8-C14; C8-12; C8-10; or C10-12. In one particularly preferred embodiment, the linear carbon chain is C8. In another particularly preferred embodiment the linear carbon chain is C11. It will be appreciated that the linear carbon chain may be an alkane, an alkene, or an alkyne, although without limitation thereto.

Without wishing to be bound by theory, it is considered that extended linear chain lengths such as C8 and C11 provide greater distance between the nanoparticle and the vancomycin structure. It is hypothesized that this may reduce the chance that the attached moiety will disrupt vancomycin activity, and/or that the vancomycin will affect the properties of the nanoparticle.

It is preferred according to the invention that the first linker is directly connected or directly bound to the glycopeptide antibiotic (see, e.g. FIG. 1).

In some embodiments of the construct of the invention, the first linker may be connected to the glycopeptide antibiotic via reaction with a moiety selected from the group consisting of: a C-terminal carboxy moiety; a primary or secondary N-terminal moiety; a hydroxyl moiety; a phenolic moiety; and an amine moiety. Preferably, the first linker is connected to the glycopeptide antibiotic via an amide group.

In preferred embodiments, the first linker comprises one or more nitrogen-containing moieties. The nitrogen-containing moieties may be any suitable moieties. The nitrogen-containing moieties may contain one or a plurality of nitrogens. In some preferred embodiments, the nitrogen-containing moieties contain between 1 and 6 nitrogens, or preferably between 1 and 3 nitrogens. The nitrogen-containing moieties may be linear or cyclic moieties, including heterocyclic moieties.

In particularly preferred embodiments, the one or more nitrogen-containing moieties include an amine-derived moiety and/or an azide-derived moiety. By amine-derived moiety and azide-derived moiety it is intended that, whatever the actual functionality of the relevant connecting bond or moiety in the construct, it was at least in part derived by the coming together of an amine or an azide with a complimentary reactive functional group. For example, an amide bond in the construct could be termed an amine-derived moiety as it may have been formed from reaction of an amine with a carboxy group. The terms cover situations where the amine or other relevant group was present on precursors to the linker, glycopeptide antibiotic or nanoparticle prior to reaction to form the final construct.

Preferably, the first linker comprises a nitrogen-containing moiety at a first end of the linker. Preferably, the moiety is an amine-derived moiety. Preferably the moiety connects the linker to the glycopeptide antibiotic. Preferably the connection is a direct connection.

It will be understood that the amine-derived moiety will be derived from an amine upon binding to another component forming part of the construct. It will be further understood that the particular identity of the amine-derived moiety will typically depend upon the component of the construct to which the amine-derived moiety is bound.

In preferred embodiments wherein the amine-derived moiety is bound to the glycopeptide antibiotic by the C-terminal carboxy moiety of the glycopeptide antibiotic, the amine-derived moiety will be an amide (see, e.g. FIG. 1).

In some embodiments wherein the amine-derived moiety is bound to the glycopeptide antibiotic by a primary or secondary amine group the amine-derived moiety will be a urea.

In some embodiments wherein the amine-derived moiety is bound to the glycopeptide antibiotic by a hydroxyl or phenolic group, the amine-derived moiety will be a urethane.

Preferably, the first linker comprises a nitrogen-containing moiety at a second end of the linker. In preferred embodiments, said moiety is an azide-derived moiety. In these embodiments, preferably, the azide-derived moiety is connected to a second linker of the construct, as hereinbelow described. Preferably the connection is a direct connection.

It will be understood that the azide-derived moiety will be derived from an azide upon binding to another component of the construct. It will be further understood that the particular identity of azide-derived moiety will typically depend upon the component of the construct to which azide-derived moiety is bound.

In some preferred embodiments the azide-derived moiety is a triazole moiety. Preferably, the triazole moiety is directly connected to a second linker of the construct (see, e.g., FIG. 1). With reference to FIG. 1, the skilled person will readily appreciate that the triazole molecule of the first linker can be formed by the reaction of an azide group of a first linker precursor (FIG. 1B) and an alkyne group of a dibenzocyclooctyl moiety of a second linker precursor (FIG. 1C).

It will further be readily understood that the triazole moiety may be alternatively formed, e.g. between an alkyne group from the second end of the first linker and an azide group of a precursor to the second linker. Preferably, the formation of the triazole from the precursors of the first and second linkers is achievable by rapid or ‘click’ reaction chemistry (see, e.g., the Examples). For a summary of click chemistry, including in the context of triazole formation, the skilled person is directed to Kolb et al (2003) Drug Discovery Today, 8(24) 1128-1137, incorporated herein by reference.

In alternative embodiments, the first linker may comprise a sulphide and/or maleimide moiety at the first end and/or the second end. In some such embodiments, the first linker is connected to a second linker via the sulphide and/or maleimide. In some such embodiments the first linker is connected to the nanoparticle via the sulphide and/or maleimide.

### Second Linkers

In particularly preferred embodiments of the construct, the first linker is connected to the nanoparticle via a second linker as hereinabove described. Preferably, the second linker is an organic molecule.

In some preferred embodiments, as hereinabove described, the second linker can be formed from a precursor comprising an alkyne moiety. In one particularly preferred such embodiment, the alkyne moiety is a strained cycloalkyne. Preferably, the cycloalkyne is a cyclooctyne. Preferably, the cyclooctyne is dibenzocyclooctyne (DBCO) (see, e.g. FIG. 1). As hereinabove described, it will be readily appreciated that an azide group of a first linker precursor may react with an alkyne group (such as that of dibenzocyclooctyne) of a second linker precursor to form a triazole moiety of the first linker, which in this case will connect the first linker and the second linker (see, e.g. FIG. 1D).

In some preferred embodiments, the second linker comprises a PEG moiety. Preferably, the PEG moiety is at least PEG3. Preferably the PEG group is in the range PEG3 to PEG10, including PEG4; PEG5; PEG6; PEG7; PEG8; and PEG9. In some particularly preferred embodiments, the PEG group is PEG3 or PEG4.

In particularly preferred embodiments wherein the second linker comprises an alkyne-derived moiety (e.g. DBCO) and a PEG moiety (e.g. PEG4), the alkyne-derived moiety is connected to the PEG moiety via an amide-containing linear organic molecule (see, e.g., FIGS. 1C and 1D). Preferably, a first amide bond connects a first end of the linear organic molecule to the alkyne-derived moiety. Preferably, a second amide bond connects a second end of the linear organic molecule to the PEG moiety.

Preferably, the PEG moiety of the second linker is bound to a coating of the nanoparticle of the construct. In a preferred embodiment, the PEG moiety is bound to a HSA coating of the nanoparticle via an amide bond (see, e.g., FIG. 1).

### Glycopeptide Antibiotic Local Density

Without limitation thereto, it will be appreciated that constructs of this aspect will typically include multiple glycopeptide antibiotic molecules bound to a single nanoparticle. In embodiments wherein the first linker connects the glycopeptide antibiotic to a second linker that is attached to the nanoparticle (or preferably a coating thereof), typically the construct will comprise multiple second linkers attached to the nanoparticle, and multiple glycopeptide antibiotics attached to the nanoparticle via respective first and second linkers.

In certain preferred embodiments, constructs of the invention have a minimum density of glycopeptides bound to the nanoparticle, or a minimum ‘local density’. In this context “local density” is defined as the number of functionally active glycopeptide antibiotic molecules per unit surface area of the nanoparticle (molecules/μm²).

In certain embodiments, constructs of the invention may have local density of between about 10 and about 100000 molecules/μm², including about: 20; 30; 40; 50; 60; 70; 80; 90; 100; 200; 300; 400; 500; 600; 700; 800; 900; 1000; 2000; 3000; 4000; 5000; 6000; 7000; 8000; 9000; 10000; 20000; 30000; 40000; 50000; 60000; 70000; 80000; and 90000.

In some embodiments, constructs of the invention have local density of 100 or less, to about 4000 molecules/μm². Local density within this range is defined as “low” local density.

In some embodiments, constructs of the invention have local density of greater than 4000 to less than 9000 molecules/μm². Local density within this range is referred to as “intermediate” local density.

In some embodiments, constructs of the invention have local density of greater than 9000 to about 20000 molecules/μm², or even greater. Local density within this range is referred to as “high” local density.

In some particularly preferred embodiments constructs of the invention have intermediate local density. In some particularly preferred embodiments, constructs of the invention have high local density.

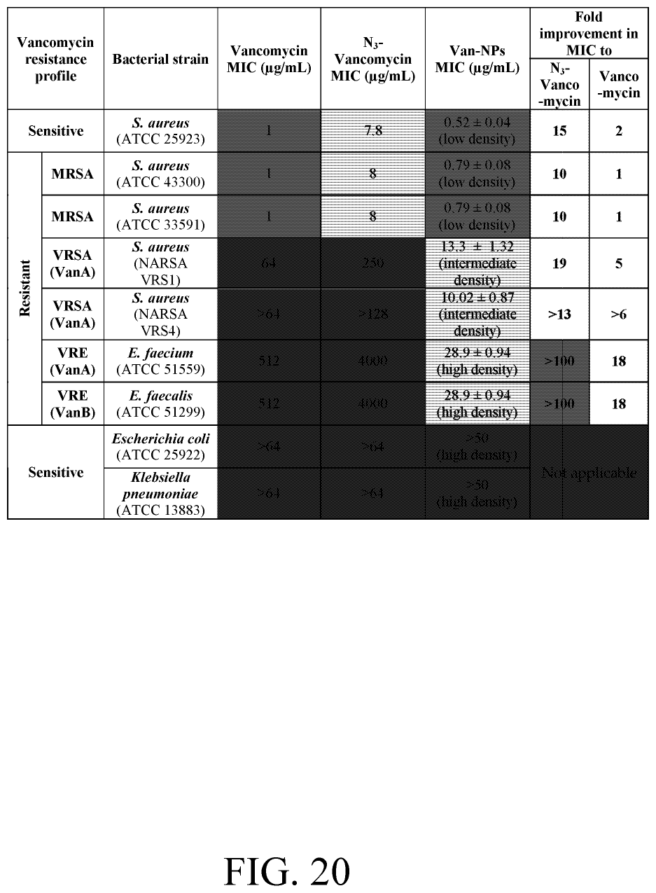

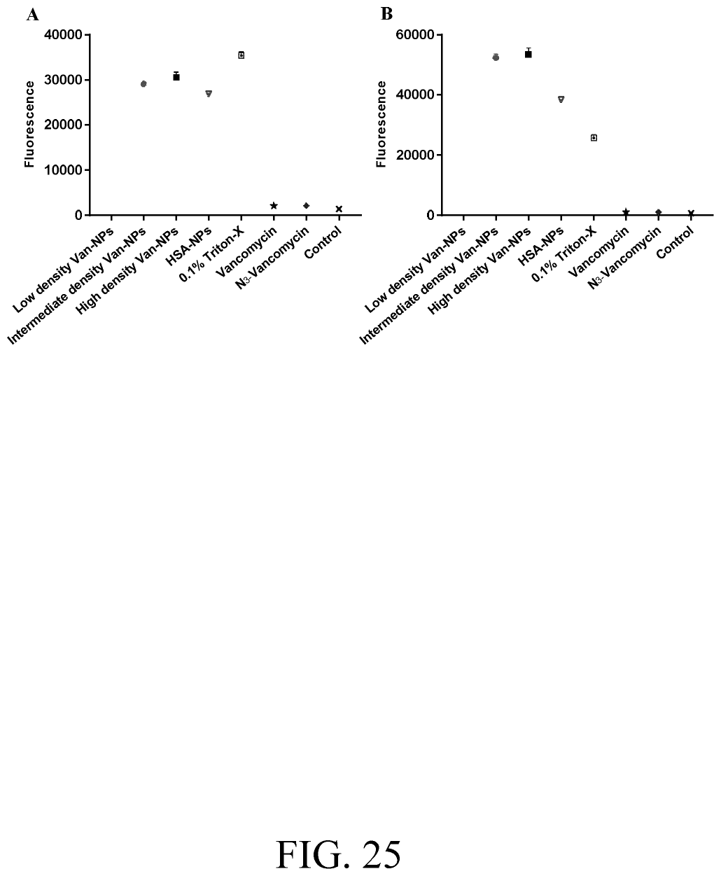

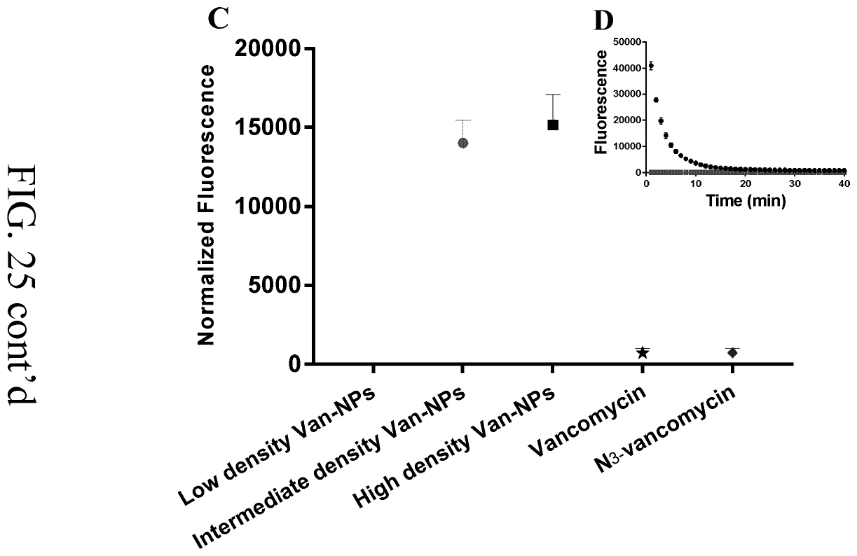

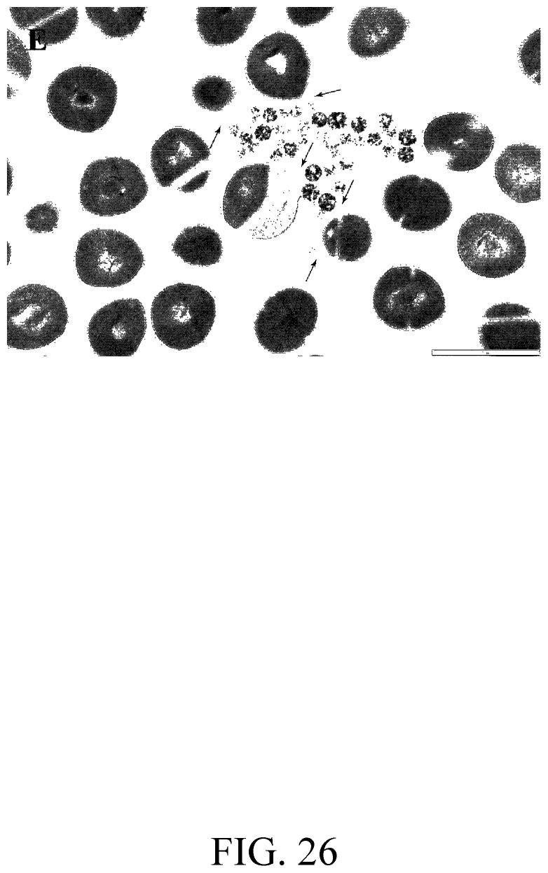

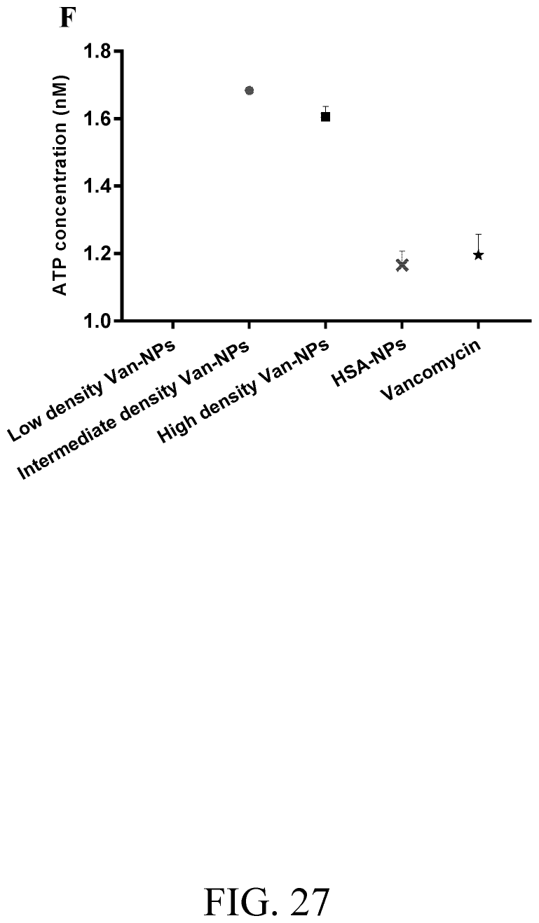

As set forth in Example 3 with reference to FIGS. 20-25, constructs with intermediate or high local density may have particularly desired characteristics with respect to binding to and/or inhibition of Gram positive bacteria.

### Methods of Bioseparation

Another aspect of the invention relates to methods of bio separation using a construct of the invention to obtain a microorganism from a sample. The method will include the steps of:

(a) combining a construct with a sample containing a microorganism or component thereof, the construct comprising (i) an optionally derivatized glycopeptide antibiotic; (ii) a nanoparticle; and (iii) a first linker connecting (i) and (ii);

(b) selectively binding the construct to the microorganism or component thereof via the glycopeptide antibiotic; and

(c) selectively obtaining the construct bound to the microorganism or component thereof from the sample via the nanoparticle, to thereby obtain the microorganism or component thereof from the sample.

The construct is preferably a bio separation construct as hereinabove described.

As hereinabove described, certain particularly preferred embodiments of the invention relate to bioseparation from human blood samples. It will be appreciated, however, that the method of this aspect is not limited thereto and may potentially be used with any suitable sample, including laboratory or environmental samples (such as contaminated water samples), in addition to biological samples including both plants and animals, as hereinabove described.

Preferably the sample of step (a) is a sample obtained from a biological subject. Preferably the subject is a human or an animal as hereinabove described. In one preferred embodiment, the sample is urine. In another preferred embodiment, the sample is blood or a blood product including platelets, plasma, and serum. Preferably, the blood or blood product is human blood.

It will be appreciated that additional reagents such as buffers may be added to the sample in step (a) to facilitate selective bacterial capture.

In some preferred embodiments wherein the sample of step (a) is a blood sample, the blood sample comprises aggregated red blood cells (RBCs) and/or white blood cells (WBCs). In these embodiments, the method will preferably further include step (ai) of aggregating blood cells in the blood sample, prior to combining the construct with the sample.

It has been determined that aggregation of blood cells in blood samples is typically required to achieve optimal bioseparation of Gram positive bacteria using constructs of the invention. In preferred embodiments, step (ai) of aggregating red and/or white blood cells includes chemical aggregation. Preferably, the chemical aggregation includes the addition of dextran polymer. In some preferred embodiments, the chemical aggregation includes the addition of glucose.

In certain embodiments, chemical aggregation as per step (ai) includes the addition of an about 10:1 to about 1:10 solution of dextran polymer supplemented with 0.1% D-glucose. Preferably the solution is an about 1:1 solution. In some embodiments, the solution may comprise other components, such as platelet additive solution (SSP+).

In some preferred embodiments, the chemical aggregation includes incubation at room temperature (˜22° C.). Preferably, the incubation is for a duration of at least: 5 minutes; 10 minutes; 15 minutes; 20 minutes; 25 minutes; 30 minutes; 35 minutes; 40 minutes; 45 minutes; 50 minutes 55 minutes; or 60 minutes. In some embodiments, the incubation is for a duration of at least: 2 hours; 3 hours; 4 hours; or 5 hours.

In some preferred embodiments, the chemical aggregation reduces total RBCs present in solution in the blood sample (i.e. non-aggregated RBCs) by at least: 70%; 80%; 90%; 95%; or 99%. In some preferred embodiments, the chemical aggregation reduces total WBCs in solution in the blood sample (i.e. non-aggregated WBCs) by at least: 70%; 80%; 90%; 95%; or 99%. In some particularly preferred embodiments the chemical aggregation reduces RBCs and WBCs in solution in the blood sample by at least: 90%; 95%; or 98%.

As set forth in the Examples, chemical aggregation has been shown to be highly effective for aggregation of blood cells prior to bioseparation using constructs of the invention. It will be further appreciated that, although physical aggregation steps (e.g. centrifugation) may also be used, such steps are not preferred as they typically require manual labour and can be difficult to automate. Furthermore, centrifugation steps may result in high levels of aggregation of bacterial cells.

As described above, preferred embodiments of the invention relate to bioseparation of Gram positive bacteria. Preferably the microorganism that is selectively bound according to step (b) of method of this aspect is a Gram positive bacteria. The Gram positive bacteria may be a pathogenic bacteria or a non-pathogenic bacteria.

It will be appreciated that, in the context of step (b) of the method of this aspect, by “selectively binding” or “selectively bound” etc., is meant that the microorganism is bound with at least partial specificity as compared to other sample components and/or microorganisms. It will be further appreciated that, as hereinabove described, glycopeptide antibiotics typically bind to Gram positive bacteria as a group selectively (as compared, for example, to Gram negative bacteria, or non-Gram staining bacteria, or mammalian cells). As such, unless the context requires otherwise, in the context of binding to Gram positive bacteria “selectively binding” etc. will refer to binding to Gram positive bacteria as a group with at least partial specificity (rather than binding to a particular Gram positive bacterial species or strain).

In preferred embodiments the microorganism bound according to step (b) is a pathogenic Gram positive bacteria. Preferably, the pathogenic Gram positive pathogenic bacteria is selected from the group consisting of: Bacillus; Clostridium; Corynebacterium; Enterococcus; Listeria; Staphylococcus; and Streptococcus. Preferably, the Gram positive bacteria is a cocci e.g. Staphylococcus; or Streptococcus.

In some preferred embodiments, the Staphylococcus is S. aureus. The S. aureus may be, without limitation thereto, a glycopeptide-sensitive strain (such as ATCC 25923); an MRSA stain (such as ATCC 43300); a glycopeptide-intermediate (GISA) strain (such as ATCC 700698); or a vancomycin-resistant (VRSA) strain (such as NARSA VRS4).

In some embodiments, the Staphylococcus is S. epidermis. The S. epidermis may be, without limitation thereto, a glycopeptide-sensitive strain (such as ATCC 12228); or glycopeptide-intermediate (GISE) strain (such as NARSA NRS60).

In some preferred embodiments wherein the Streptococcus is S. pneumoniae. The S. pneumoniae may be, without limitation thereto, a glycopeptide sensitive strain (such as ATCC 33400); or a glycopeptide resistant strain (such as ATCC 700677).

In some embodiments, the Enterococcus is E. faecium. The E. faecium may be, without limitation thereto, a vancomycin A (Van A) resistant strain (such as ATCC 51559) or a vancomycin B (Van B) resistant strain (such as ATCC 51299).

Preferably, step (c) of the method of this aspect is performed using a magnetic field to magnetically capture magnetic separation nanoparticles in the construct bound to the microorganism. Techniques for capture of magnetic nanoparticles from a sample have been described extensively in the art. The skilled person is directed to ‘Ex-Vivo Application of MNPs’ in Magnetic Nanoparticles: From Fabrication to Clinical Applications (CRC Press, 2012, Nguyen T K Thanh Ed.), which is incorporated herein by reference. Generally, for magnetic capture, an external magnetic field attractive to the magnetic nanoparticles is applied to a sample containing the magnetic nanoparticles. The external magnetic field thereby attracts the magnetic nanoparticles, stabilizing the nanoparticles against the magnetic field. Other components of the sample can then be removed, typically by physical means such as tipping, suction, or washing (e.g. with a suitable buffer).

It will be appreciated that separation of the separation nanoparticles according to step (c) of this aspect facilitates separation of the microorganism (e.g. pathogenic Gram positive bacteria) from the sample (e.g. human blood or plasma sample). Once separated according to the method of this aspect, the microorganism can potentially be used for any suitable downstream application. Suitable applications include, without limitation, analyses as hereinbelow described and/or laboratory culturing to propagate the microorganism, where such is desirable.

### Methods of Analysing a Microorganism

In a further aspect, the invention provides a method of analysing a microorganism or a component thereof in a sample, the method including the steps of:

(a) combining a construct with the sample containing a microorganism or component thereof, the construct comprising (i) an optionally derivatized glycopeptide antibiotic; (ii) a nanoparticle; and (iii) a first linker connecting (i) and (ii);

(b) selectively binding the construct to the microorganism or component thereof via the glycopeptide antibiotic;

(c) selectively obtaining the construct bound to the microorganism or component thereof from the sample via the nanoparticle to thereby obtain the microorganism or component thereof from the sample; and

(d) analysing the microorganism or component thereof.

Suitably, steps (a) to (c) according to the method of this aspect are as described above in regard to methods of bioseparation according to the invention. As described above, the microorganism is preferably a Gram positive bacteria, preferably a pathogenic Gram positive bacteria, although without limitation thereto. The sample is preferably a human blood or plasma sample, although without limitation thereto.

It will be understood that the analysis that is performed according to the method of this aspect can be any desired analysis. By way of non-limiting example, the analysis may be a biochemical analysis (e.g. determination of the composition of the microorganism); a visual analysis (e.g. electron microscopy of the microorganism); or a genetic analysis (e.g. nucleic acid sequence or gene expression analysis of the microorganism).

It is particularly preferred according to the method of this aspect that the analysis includes identification of the microorganism.

Typically, although without limitation thereto, the method of this aspect includes the further step of removing the microorganism or component thereof from the bioseparation construct prior to analysis.

In some preferred embodiments, separation of the microorganism or component thereof from the construct according to this aspect involves decreasing pH and/or heat treating the construct. In some preferred embodiments, decreasing pH is performed by addition of a weak acid, e.g. acetic acid; oxalic acid; phosphoric acid; nitrous acid; hydrofluoric acid; and methanoic acid, although without limitation thereto. Preferably, the acid is acetic acid. In preferred embodiments the pH is reduced by between about 1 and about 3 pH units, including about 2. In one particularly preferred embodiment the pH is reduced from about 7 to about 4 or about 5.

In some preferred embodiments, the temperature of heat treatment is between about 50° C. to about 80° C., including about: 55° C.; 60° C.; 65° C.; 70° C.; and 75° C. Preferably, the heat treatment is about 65° C.

In some preferred embodiments, the duration of heat treatment is between about 5 minutes and about 30 minutes, including about: 10 minutes; 15 minutes; 20 minutes; and 25 minutes. Preferably, the heat treatment is about 20 minutes.

Additionally or alternatively, separation of the microorganism or component thereof from the construct may involve sonication, as is well known in the art.

In some embodiments, the method of this aspect may include the further step of lysing the microorganism to release internal components, prior to analysis. Lysing of the microorganism prior to analysis will be typically be required for nucleic acid analysis as described below.

It will be appreciated that, in embodiments wherein the microorganism is analysed by nucleic acid analysis, the method may include further steps of DNA capture and/or DNA purification, as hereinbelow described. In a particularly preferred form wherein the microorganism is analysed by nucleic acid analysis, the method may include further steps of complete bacterial lysis (e.g. using heating at 65° C. for 20 min), bacterial protein denaturation (e.g. using guanidine hydrochloride), and DNA capture using magnetic nanoparticles and/or magnetic DNA purification.

In one particularly preferred embodiment, analysis according to the method of this aspect is performed by mass spectrometry. A preferred form of mass spectrometry for analysis of the microorganism according to this aspect is using a matrix-assisted laser desorption/ionization (MALDI) mass spectrometry technique. Typically, the MALDI technique will be a MALDI-time of flight (TOF) technique, although without limitation thereto. As will be understood by the skilled person, during MALDI analysis samples are deposited on a surface, incorporated into crystals of a codeposited matrix, and ions are desorbed directly into a gas phase by interaction with a pulsed laser beam. The ions are then analysed using a suitable mass spectrometer. For a summary of MALDI and MALDI-TOF, the skilled person is directed to Karas et al. (2003) Chemical Reviews. 103(2) 427-440, which is incorporated herein by reference.

MALDI mass spectrometry approaches are commonly used for protein identification and characterization. By “protein” is meant an amino acid polymer. Amino acids may be natural or non-natural amino acids, D- or L-amino acids as are well understood in the art. In some embodiments, protein characterization of the microorganism by MALDI or MALDI-TOF mass spectrometry is used for identification of the microorganism. Various approaches for identification of microorganisms such as Gram positive bacteria using MALDI have been developed. It will be appreciated that approaches may involve either intact microbial cells, or microbial cell extracts. For a review of such approaches, the skilled person is directed to Singhal et al. (2015) Frontiers in Microbiology. 6 791, which is incorporated herein by reference. MALDI mass spectrometry may also be used for identification of resistance profiles of microorganisms analysed according to this aspect.

In another particularly preferred embodiment, analysis according to the method of this aspect is nucleic acid analysis. The term “nucleic acid” as used herein designates single- or double-stranded DNA and RNA. DNA includes genomic DNA and cDNA. RNA includes mRNA, RNA, RNAi, siRNA, cRNA and autocatalytic RNA. Nucleic acids may also be DNA-RNA hybrids. A nucleic acid comprises a nucleotide sequence which typically includes nucleotides that comprise an A, G, C, T or U base. However, nucleotide sequences may include other bases such as inosine, methylycytosine, methylinosine, methyladenosine and/or thiouridine, although without limitation thereto.

The nucleic acid analysis may be nucleotide sequencing, or gene expression analysis, as are well known in the art. Nucleic acid sequencing is particularly desirable for microorganism identification according to the method of this aspect.

As will be readily understood by the skilled person, a variety of techniques for nucleic acid sequencing exist. These include Sanger sequencing (Sanger et al. (1977) Proceedings of the National Academy of Sciences. 74(12) 5463-5467) and automated versions thereof, and newer technologies which are typically referred to as ‘Next Generation’ sequencing techniques (Mardis (2013) Annual Review of Analytical Chemistry. 6 287-303). Recently, nanopore sequence, particularly the Oxford

Nanopore systems (including the ‘MinION’) have seen substantial assessment and optimization for nucleotide sequencing. The skilled person is directed to Lu et al (2016) Genomics, Proteomics & Bioinformatics. 14(5) 265-279 for an overview of sequencing with the Oxford Nanopore MinION system.

It is particularly preferred according to embodiments of this method wherein the microorganism is identified using nucleotide sequencing that the nucleotide sequencing is sequencing using a Next Generation technique, such as nanopore sequencing.

Characteristics of nanopore sequencing, and the MinION system in particular (including relatively small size and capacity for rapid sequencing and ‘real-time’ analysis) may make it particularly desirable for diagnostic analyses, for example identification of Gram positive bacteria in a human blood sample (the skilled person is directed to Pennisi (2016) Science. 351(6275) 800-801, incorporated herein by reference, for a summary in this regard). Therefore, in one particularly preferred embodiment, the nucleotide sequencing is nanopore sequencing. Preferably, the nanopore sequencing is sequencing using a portable nanopore sequencer, such as the MinION system, although without limitation thereto.

It will be understood that both DNA and RNA analysis is suitable according to the method of this aspect. Typically, RNA sequence analysis (sometimes referred to as ‘RNA-seq’) is performed by sequencing of cDNA produced from an RNA template. It will be appreciated that, in addition to nucleotide identification, RNA or cDNA sequencing can be used for gene expression analyses, where this is desirable.

It will be further appreciated that nucleic acid analysis according to this method may involve nucleic acid sequence amplification. As used herein, a “nucleic acid sequence amplification” includes but is not limited to techniques such as polymerase chain reaction (PCR) as for example described in Chapter 15 of CURRENT PROTOCOLS IN MOLECULAR BIOLOGY Eds. Ausubel et al. (John Wiley & Sons NY USA 1995-2001) strand displacement amplification (SDA); rolling circle replication (RCR) as for example described in International Application WO 92/01813 and International Application WO 97/19193; nucleic acid sequence-based amplification (NASBA) as for example described by Sooknanan et al. 1994, Biotechniques 17 1077; ligase chain reaction (LCR) as for example described in International Application WO89/09385 and Chapter 15 of CURRENT PROTOCOLS IN MOLECULAR BIOLOGY supra; Q-β replicase amplification as for example described by Tyagi et al., 1996, Proc. Natl. Acad. Sci. USA 93 5395 and helicase-dependent amplification as for example described in International Publication WO 2004/02025.

Certain Next Generation sequencing techniques involve nucleic acid sequence amplification prior to nucleic sequencing. Particular sample preparation techniques applicable for various Next Generation sequencing approaches are known and have been extensively described, for example in manufacturer instructions for sample preparation kits available for proprietary sequencing technologies of Illumina (see, http://www.illumina.com/techniques/sequencing/ngs-library-prep.html); Pacific Biosystems (http://www.pacb.com/products-and-services/consumables/pacbio-rs-ii-consumables/sample-and-template-preparation-kits/); and Applied Bio systems (https://www.neb.com/applications/library-preparation-for-next-generation-sequencing/ion-torrent-dna-library-preparation).

It will be further appreciated that, although Next Generation sequencing techniques are typically preferred for nucleic sequence analysis according to the invention, other techniques for nucleic acid analysis may also be suitable. Such techniques include diagnostic PCR and RT-PCR (including real-time RT-PCR or qPCR, which can be used for nucleic acid quantification and/or gene expression analysis) with primers targeting a microorganism of interest, as are well known in the art.

In another embodiment, analysis according to this aspect may be a fluorescence-based analysis.

Preferably, in these embodiments, the method includes the further step of binding a fluorescent tag or probe to the microorganism or component thereof. The fluorescent tag or probe will preferably include a fluorescent molecule or fluorescent nanoparticle connected to a microorganism-specific ligand.

In an embodiment, the fluorescent nanoparticle comprises a fluorescent dye. In particularly preferred embodiments, the fluorescent nanoparticle is or comprises quantum dots. For a review of the application of quantum dots to fluorescent detection of biological materials, the skilled person is directed to Medintz et al (2005) Nature Materials, 4 435-446, which is incorporated herein by reference.

The microorganism-specific ligand may be any suitable ligand. In a preferred embodiment, the microorganism-specific ligand is an antibody.

Generally, in these embodiments, in addition to binding of the construct of the invention, the presence of a suitable microorganism or component results in binding of the fluorescent tag or probe to the microorganism or component thereof. A fluorescent signal from the tag or probe can thereby be used for analysis (e.g. identification) of the microorganism or component thereof.

It will be understood that, according to these embodiments, the fluorescent tag or probe can be combined with the microorganism or component thereof and construct of the invention at any suitable stage, e.g. during or before any of steps (a)-(c), or after step (c).

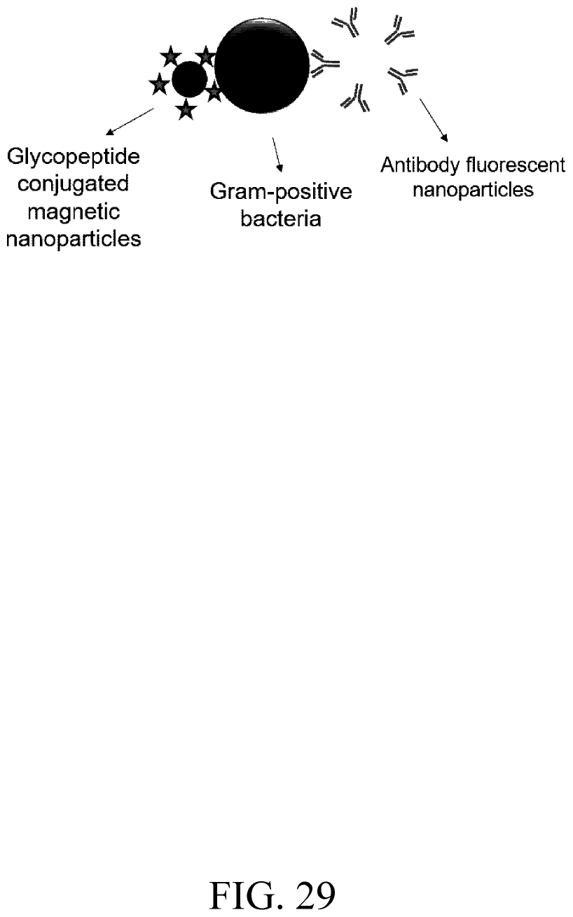

A schematic of a construct of the invention bound to a Gram positive bacteria bound to a fluorescent tag is provided in FIG. 29.

It will be further appreciated that analysis according to this aspect may involve identification of a resistance profile of the microorganism, to facilitate appropriate treatment (e.g. antibiotic treatment). Techniques for determining antibiotic resistance profiles are well known in the art. By way of example, the skilled person is directed to Reller et al (2009) Clinical Infectious Disease, 49(11) 1749-1755, incorporated herein by reference.

Certain exemplary embodiments of microorganism capture and analysis falling within the scope of the this aspect of the invention are described in Hassan et al (2018) Biosensors and Bioelectronics, 99 (2018) 150-155, incorporated herein by reference.

### Method of Screening a Sample or Product

In a further aspect, the invention provides a method of screening a sample for the presence of a microorganism or component thereof of interest, the method including the steps of:

(a) combining a construct with a sample, the construct comprising (i) an optionally derivatized glycopeptide antibiotic; (ii) a nanoparticle; and (iii) a first linker connecting (i) and (ii); and,

(b) selectively obtaining the construct from the sample using the nanoparticle; and

(c) performing analysis to determine if a microorganism or component thereof of interest is or was bound to the construct,

wherein a determination that the microorganism or component thereof of interest is or was bound to the construct indicates that the sample contains the microorganism or component thereof of interest, and a determination that the microorganism or component thereof of interest is not or was not bound to the construct indicates that the sample does not contain the microorganism or component thereof of interest.

A sample according to this aspect may be any suitable sample as hereinabove described. In certain preferred embodiments, screening according to this aspect is for the purposes of assessing microorganism contamination of a product that is to be administered to a subject, such as a human or animal subject. Such products may be any of a broad range of products, including, for example, food products; cosmetic products; and pharmaceutical or medical products. In certain embodiments, the sample may be processed to remove components interfering with binding of bacteria to the glycopeptide antibiotic of the construct, prior to screening.

In one particularly preferred embodiment, the sample according to this aspect is blood or a blood product. In one preferred embodiment, the sample according to this aspect is a plasma product. In one preferred embodiment, the sample according to this aspect is a platelet product.

In embodiments wherein the sample is blood or a blood product containing red and/or white blood cells, preferably the red and/or white blood cells will be aggregated prior to step (a).

### Methods of Diagnosing a Disease or Condition

Another aspect of the invention is directed to a method of diagnosing a disease or condition. The method will include the steps of analysing a microorganism in a sample of a subject to identify the pathogen, as described for the directly preceding aspect, and diagnosing a disease or condition based on the identity of the microorganism.

Preferably the disease or condition is an infection with a Gram positive bacteria. Preferably, the pathogenic Gram positive pathogenic bacteria is selected from the group consisting of: Bacillus; Clostridium; Corynebacterium; Enterococcus; Listeria; Staphylococcus; and Streptococcus. Preferably, the Gram positive bacteria is a cocci e.g. Staphylococcus; or Streptococcus.

In some preferred embodiments, the Staphylococcus is S. aureus. The S. aureus may be, without limitation thereto, a glycopeptide-sensitive strain (such as ATCC 25923); an MRSA stain (such as ATCC 43300); a glycopeptide-intermediate (GISA) strain (such as ATCC 700698); or a vancomycin-resistant (VRSA) strain (such as NARSA VRS4).

In some embodiments, the Staphylococcus is S. epidermis. The S. epidermis may be, without limitation thereto, a glycopeptide-sensitive strain (such as ATCC 12228); or glycopeptide-intermediate (GISE) strain (such as NARSA NRS60).

In some preferred embodiments wherein the Streptococcus is S. pneumoniae. The S. pneumoniae may be, without limitation thereto, a glycopeptide sensitive strain (such as ATCC 33400); or a glycopeptide resistant strain (such as ATCC 700677).

In some embodiments, the Enterococcus is E. faecium. The E. faecium may be, without limitation thereto, a vancomycin A (Van A) resistant strain (such as ATCC 51559) or a vancomycin B (Van B) resistant strain (such as ATCC 51299).

In certain embodiments, the disease or condition may be selected from the group consisting of a bacterial infection of: the respiratory system (e.g. pneumonia); the digestive tract (e.g. gastroenteritis); the sinus (e.g. sinusitis); the ears (e.g. otitis media); the nervous system (e.g. meningitis); the skin (e.g. cellulitis); or the endocrine system (e.g. bacterial pancreatitis).

In one particularly preferred embodiment the disease or condition is bacteraemia. In another particularly preferred embodiment the disease or condition is bacterial sepsis. In one preferred embodiment, the disease or condition is bacterial sepsis caused by S. aureus.

In another particularly preferred embodiment, the disease or condition is a urinary tract infection.

Preferably the subject is a human or an animal subject as hereinabove described. In particularly preferred embodiments the subject is a human.

As described above, current methods for diagnosis of bacteraemia and/or bacterial sepsis (and also many other bacterial diseases) typically rely on bacterial culture. Methods involving bacterial culture can have significant disadvantages, including substantial periods of time for culture and diagnosis, for examples 10-24 hours, and relatively high rates of false positives.

Embodiments of the invention may have particular advantages with respect to time to identify a microorganism or component thereof in a sample and/or to diagnosis a disease or condition based on this identification.

In some preferred embodiments, identification of a microorganism or component thereof as per the directly preceding aspect of the invention can be performed in less than 10 hours; less than 9 hours; less than 8 hours; less than 7 hours; less than 6 hours; less than 5 hours; less than 4 hours; less than 3 hours; less than 2 hours; or less than 1 hour.

In some particularly preferred embodiments, diagnosis of a disease or condition according to the method of this aspect can be performed in in less than 10 hours; less than 9 hours; less than 8 hours; less than 7 hours; less than 6 hours; less than 5 hours; less than 4 hours; less than 3 hours; less than 2 hours; or less than 1 hour.

Embodiments of the invention may have particular advantages with respect to false positive rate for identification of a microorganism in a sample and/or diagnosis of a disease or condition based on this identification.

In some preferred embodiments, identification of a microorganism as per the directly preceding aspect has a false positive rate of less than 10%; less than 9%; less than 8%; less than 7%; less than 6%; less than 5%; less than 4%; less than 3%; less than 2%; or less than 1%.

In some preferred embodiments, diagnosis of a disease or condition according to the method of this aspect has a false positive rate of less than 10%; less than 9%; less than 8%; less than 7%; less than 6%; less than 5%; less than 4%; less than 3%; less than 2%; or less than 1%.

A related aspect of the invention provides a method of treating a disease or condition, the method including the steps of diagnosing a disease or condition as hereinabove described, and treating the disease or condition based on the diagnosis.

It will be appreciated that the particular form of treatment that will be suitable according to the method of this aspect will be related to the particular disease or condition that is diagnosed. The particular form of treatment may also be related to the antibiotic resistance profile of the microorganism responsible for the disease or condition, where this is determined (e.g. by analysis as herein described, or any other suitable method).

Typically, in preferred embodiments wherein the disease is a Gram positive bacterial infection, the treatment will comprise administration of an antibiotic. In some preferred embodiments, the antibiotic is selected from the group consisting of cloxacillin; dicloxacillin; methlocillin; nafcillin; oxacillin; cefazolin; cefoxitin; cefuroxime; cefepime; cefoperazone; cefotaxime; ceftazidime; ceftizoxime; ceftriaxone; trimethoprim; sulfamethoxazole; amoxicillin; clavulanate; penicillin; penicillin G; streptomycin; amoxicillin; clindamycin; doxycycline; etronidazole; rifampin; linezolid; daptomycin; dalbavancin; oritavancin; telavancin; tedizolid; ceftaroline; and vancomycin, or combinations thereof. In some particularly preferred embodiments the antibiotic is vancomycin.

In certain preferred embodiments wherein the disease or condition is a S. aureus infection, the antibiotic is selected from the group consisting of trimethoprim; sulfamethoxazole; clindamycin; vancomycin; doxycycline; minocycline; linezolid; rifampin; daptomycin; dalbavancin; oritavancin; telavancin; tedizolid; ceftaroline; or combinations thereof.

The method of this aspect may have particular advantages with respect to the time taken for diagnosis and initial treatment, e.g. initial administration of an antibiotic. In some preferred embodiments, diagnosis and initial treatment according to the method of this aspect can be performed in less than 10 hours; less than 9 hours; less than 8 hours; less than 7 hours; less than 6 hours; less than 5 hours; less than 4 hours; less than 3 hours; less than 2 hours; or less than 1 hour.

### Glycopeptide Antibiotic-Linker Compounds

The invention also provides compounds or ‘adducts’ comprising an optionally derivatized glycopeptide antibiotic bound to a first linker. Such adducts of the invention will be suitable for connection to a nanoparticle, to form constructs as hereinabove described.

Preferably, the glycopeptide antibiotic of such adducts is selected from the group consisting of vancomycin; teicoplanin; oritavancin; telavancin; chloroeremomycin; and balhimycin. Preferably the glycopeptide antibiotic is vancomycin.

The first linker is preferably a first linker as hereinabove described.

Preferably, the first linker of this form of the invention comprises a PEG moiety, preferably at least PEG3. Preferably the PEG group is in the range PEG3 to PEG-10, including PEG4; PEG5; PEG6; PEG7; PEG8; and PEG9. In some particularly preferred embodiments, the PEG group is PEG3 or PEG4.

In some embodiments, the linker comprises a linear carbon chain, preferably wherein the linear carbon chain is C4-C15, such as C5; C6; C7; C8; C9; C10; C11; C12; C13; and C14. In particularly preferred embodiment the linear carbon chain is C8. In other particularly preferred embodiment the linear carbon chain is C11.

In some embodiments, the linker may be connected to the glycopeptide antibiotic via a moiety selected from the group consisting of: a C-terminal carboxy moiety; a primary or secondary N-terminal moiety; a hydroxyl moiety; a phenolic moiety; and a vancosamine moiety.

Preferably, the linker is connected to the glycopeptide antibiotic via an amide bond.

In preferred embodiments, the linker comprises one or more nitrogen-containing moieties. Preferably, the one or more nitrogen-containing moieties include moieties selected from the group consisting of an amine-derived moiety; an azide moiety; an azide-derived moiety; and a triazole moiety.

Preferably, the linker comprises a nitrogen-containing moiety at a first end of the molecule. Preferably, the moiety is an amine-derived moiety. Preferably the moiety connects the linker to the glycopeptide antibiotic.

Preferably, the linker comprises a nitrogen-containing moiety at a second end of the molecule. In some embodiments, the moiety is an azide or azide-derived moiety.

In some preferred embodiments the adduct of this aspect further comprises a second linker as hereinabove described.

### Vancomycin-Linker-Vancomycin Compounds

The invention further provides for dimer compounds wherein a first optionally derivatized vancomycin is connected by a linker to a second optionally derivatized vancomycin.

More specifically, the invention provides for an adduct of the directly preceding aspect wherein the glycopeptide antibiotic of the compound is vancomycin, connected to a further vancomycin moiety. In preferred embodiments, the dimer compound of this aspect of the invention comprises two connected adducts of the directly preceding aspect, wherein the glycopeptide antibiotics of the respective compounds are vancomycin.

In certain embodiments, the two connected compounds are connected via a moiety comprising a linear carbon chain and/or PEG moiety. In certain embodiments the moiety is a linear carbon chain which comprises amide bonds on respective ends.

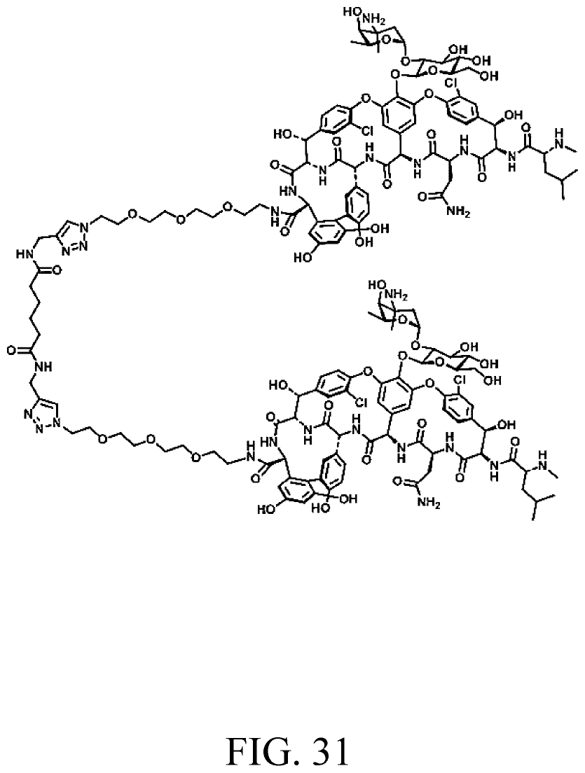

One particularly preferred embodiment of this aspect is set forth in FIG. 31

### Methods of Increasing the Activity of a Glycopeptide Antibiotic

In another aspect, the invention provides a method of increasing or enhancing the activity or efficacy of a glycopeptide antibiotic, the method including the step of connecting the glycopeptide antibiotic to a first linker. The method may optionally include the further step of connecting the glycopeptide antibiotic to nanoparticle via the first linker. In preferred embodiments, the glycopeptide antibiotic, nanoparticle, and first linker are as hereinabove described in relation to constructs of the invention.

Suitably, connecting the glycopeptide antibiotic to the first linker forms an adduct as hereinabove described. Suitably, connecting the glycopeptide antibiotic to a nanoparticle forms a construct as hereinabove described.

In preferred embodiments, the glycopeptide antibiotic is selected from the group consisting of vancomycin; teicoplanin; oritavancin; telavancin; chloroeremomycin; and balhimycin. In one particularly preferred embodiment, the glycopeptide antibiotic is vancomycin.

Preferably the activity or efficacy of the glycopeptide antibiotic is towards a Gram positive bacteria. Preferably the bacteria is a pathogenic Gram positive bacteria. Particular preferred Gram positive bacteria according to this aspect are as described above in relation to methods of diagnosing a disease or condition according to the invention.

In certain embodiments the Gram positive bacteria is a vancomycin sensitive or resistant bacteria. The vancomycin resistant bacteria may be a Van A, Van B, Van C, Van D or Van E resistant bacteria. Preferably the vancomycin resistant bacteria is selected from the group consisting of S. aureus; E. faecium; and E. faecalis. In certain embodiments, the microorganism shows at least partial resistance to the free or unconnected glycopeptide antibiotic.

As set forth below, it has been surprisingly found that connecting a glycopeptide a linker, or connecting a glycopeptide to a nanoparticle via a linker, can result in substantial decreases of minimum inhibitory concentration (MIC) of the antibiotic, and/or increases in binding avidity or strength to bacterial membranes and bacterial membrane permeability.

In some preferred aspects, the increase or enhancement of the activity or efficacy or the antibiotic is a decrease in the MIC of the antibiotic for a particular microorganism, such as a Gram positive microorganism as described above.

In some preferred embodiments, the decrease in MIC is a fold decrease of at least 2 to about 50 relative to the free or unconnected antibiotic, including a fold decrease of at least: 5; 10; 15; 20; 25; 30; 35; 40; or 45.

In preferred embodiments wherein the method does not include the optional step of connecting the glycopeptide antibiotic to the nanoparticle via the linker, the linker comprises a C8 linear carbon chain.

In preferred embodiments wherein the method includes the optional step of connecting the glycopeptide antibiotic to the nanoparticle via the linker, the linker comprises a C8 linear carbon chain, or a PEG3 moiety.

### Methods of Inhibiting a Microorganism

Another aspect of the invention provides a method of inhibiting, controlling, or killing a microorganism, the method including the steps of contacting a microorganism with a construct, adduct, or dimer of the invention as hereinabove described, to thereby inhibit, control, or kill the microorganism.

Preferably the microorganism is a Gram positive bacteria. Preferably, the Gram positive bacteria is a pathogenic Gram positive bacteria. Particular preferred Gram positive bacteria according to this aspect are as described above in relation to methods of diagnosing a disease or condition according to the invention.

In certain embodiments the Gram positive bacteria is a vancomycin resistant bacteria. The vancomycin resistant bacteria may be a Van A, Van B or Van C, Van D or Van E resistant bacteria. Preferably the vancomycin resistant bacteria is selected from the group consisting of S. aureus; E. faecium; and E. faecalis. In certain embodiments, the microorganism shows at least partial resistance to the free or unconnected glycopeptide antibiotic.

### Compositions and Methods for Treatment or Prevention of Disease

In a further aspect, the invention provides a composition for treating or preventing a disease, disorder, or condition in a subject, the composition comprising a construct, adduct, or dimer of the invention as hereinabove described.

The composition may suitably contain one or more pharmaceutically-acceptable carriers, diluents or excipients. By “pharmaceutically-acceptable carrier, diluent or excipient” is meant a solid or liquid filler, diluent or encapsulating substance that may be safely used in systemic administration.

Depending upon the particular route of administration, a variety of carriers, well known in the art may be used. These carriers may be selected from a group including sugars, starches, cellulose and its derivatives, malt, gelatine, talc, calcium sulphate, vegetable oils, synthetic oils, polyols, alginic acid, phosphate buffered solutions, emulsifiers, isotonic saline and salts such as mineral acid salts including hydrochlorides, bromides and sulphates, organic acids such as acetates, propionates and malonates and pyrogen-free water. A useful reference describing pharmaceutically acceptable carriers, diluents and excipients is Remington's Pharmaceutical Sciences (Mack Publishing Co. N.J. USA, 1991) which is incorporated herein by reference.

Dosage forms of compositions according to this aspect include tablets, dispersions, suspensions, injections, solutions, oils, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Compositions of the present invention suitable for enteral, oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

Also provided is a method of treating or preventing a disease, disorder, or condition in a subject in need thereof, the method including the step of administering to the subject an effective amount of the construct, adduct, dimer, or composition of invention, as hereinabove described.

Any safe route of administration may be employed for providing a patient with constructs or compositions of the invention. For example, enteral, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed.

The constructs or compositions may be administered in a manner compatible with the dosage formulation, and in such amount as is pharmaceutically-effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

It will also be appreciated that treatment methods and pharmaceutical compositions may be applicable to prophylactic or therapeutic treatment of mammals, inclusive of humans and non-human mammals such as livestock (e.g. horses, cattle and sheep), companion animals (e.g. dogs and cats), laboratory animals (e.g. mice, rats and guinea pigs) and performance animals (e.g. racehorses, greyhounds and camels), although without limitation thereto.

Preferably the disease or condition according to these embodiments is caused by a Gram positive bacteria. Particular preferred Gram positive bacteria according to this aspect are as described above in relation to methods of diagnosing a disease or condition according to the invention.

In certain embodiments, the disease or condition may be selected from the group consisting of a bacterial infection of: the respiratory system (e.g. pneumonia); the digestive tract (e.g. gastroenteritis); the sinus (e.g. sinusitis); the ears (e.g. otitis media); the nervous system (e.g. meningitis); the skin (e.g. cellulitis); the endocrine system (e.g. bacterial pancreatitis); or the urinary tract (e.g. a urinary tract infection).

In one particularly preferred embodiment the disease or condition is bacteraemia. In another particularly preferred embodiment the disease or condition is bacterial sepsis. In these embodiments, preferably, the disease or condition is bacterial sepsis caused by a S. aureus or S. epidermidis.

In another particularly preferred embodiment, the disease or condition is a urinary tract infection. In preferred embodiments, the disease or condition is bacterial sepsis caused by a S. aureus or S. epidermidis. In these embodiments, preferably, the disease or condition is a urinary tract infection caused by a S. aureus or S epidermidis.

### EXAMPLES

### Example 1. Production of Constructs

Constructs according to the invention were produced as depicted in FIG. 1. Vancomycin attached to an azido-PEG3-amine (N₃-PEG3-NH₂) first linker was synthesized via amide bond formation between the C-terminal carboxylate of vancomycin and the amine of the azido-PEG3-amine, with the adduct referred to hereinafter as N₃-PEG3-Van. N₃-PEG3-Van was then coupled to a magnetic nanoparticle via a second linker as hereinbelow described. Each step of the molecular architecture was carefully characterized to ensure consistent coupling of vancomycin to the nanoparticle, as described below.

### Synthesis of N₃-PEG3-Van

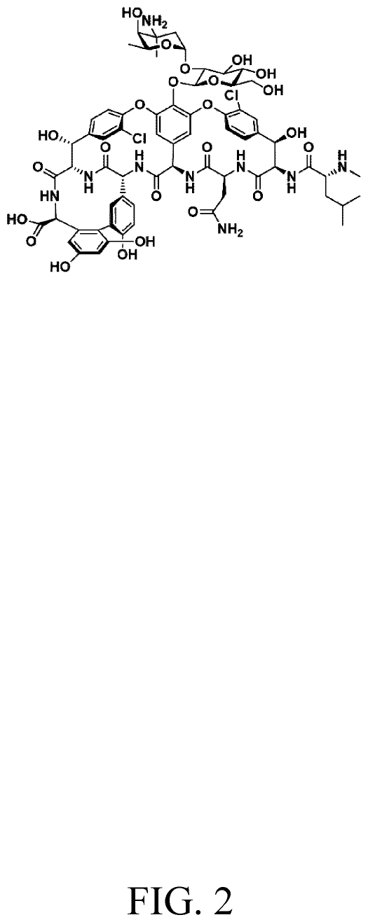

The ligand-binding region of vancomycin resides in the heptapeptide backbone; therefore, it was recognized that a first linker could potentially be added to a number of regions outside of this area to facilitate coupling onto the nanoparticles without substantially impairing potency of binding of vancomycin (FIG. 2). Sites with accessible functional groups include the C-terminal carboxy group, vancosamine and N-terminal Leu primary and secondary amine groups, and hydroxyl and phenolic groups, with all of these sites previously used to generate vancomycin derivatives.

For this example, N₃-PEG3-Van was synthesized by amide coupling between N₃-PEG3-NH₂ and the vancomycin C-terminal carboxyl group, reacting vancomycin hydrochloride with N₃-PEG3-NH₂ in the presence of benzotriazol-1-yl-oxytripyrrolidinophosphonium hexafluorophosphate (PyBOP) and N,N-diisopropylethylamine (DIPEA) in DMF. Specifically, vancomycin hydrochloride (567 mg, 3.82×10⁻⁴ mol), PyBOP (219 mg, 4.20×10⁻⁴ mol) and N₃-PEG3-NH₂ (N₃(CH₂CH₂O)₃CH₂CH₂NH₂, 100 mg, 4.58×10⁻⁴ mol) in dimethylformamide (DMF) (30 mL) were stirred until dissolved. N,N-Diisopropylethylamine (DIPEA) (532 μL, 3.05×10⁻³ mol) was added slowly to the reaction mixture. The reaction was stirred at room temperature for 2 hours, followed by removal of solvent under reduced pressure. Other standard amide bond couplings conditions can also be used (see FIG. 1)

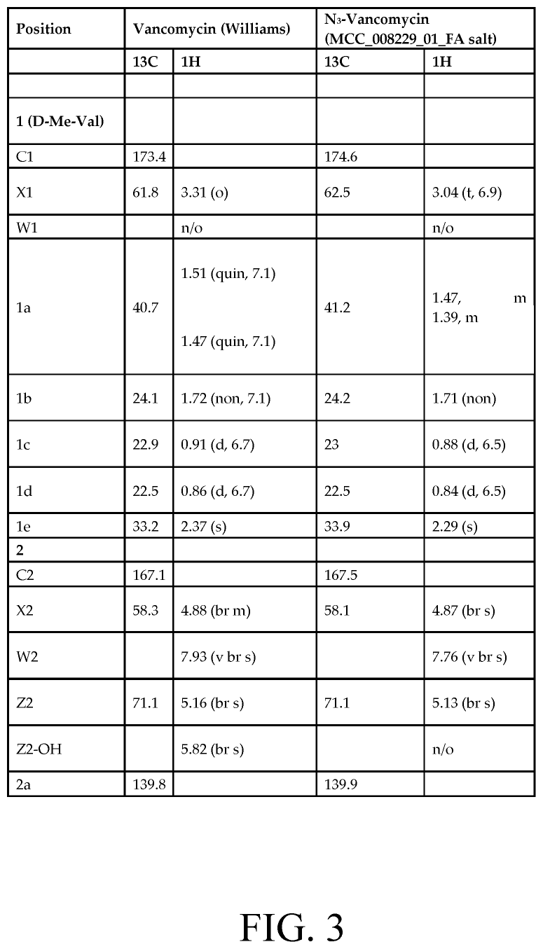

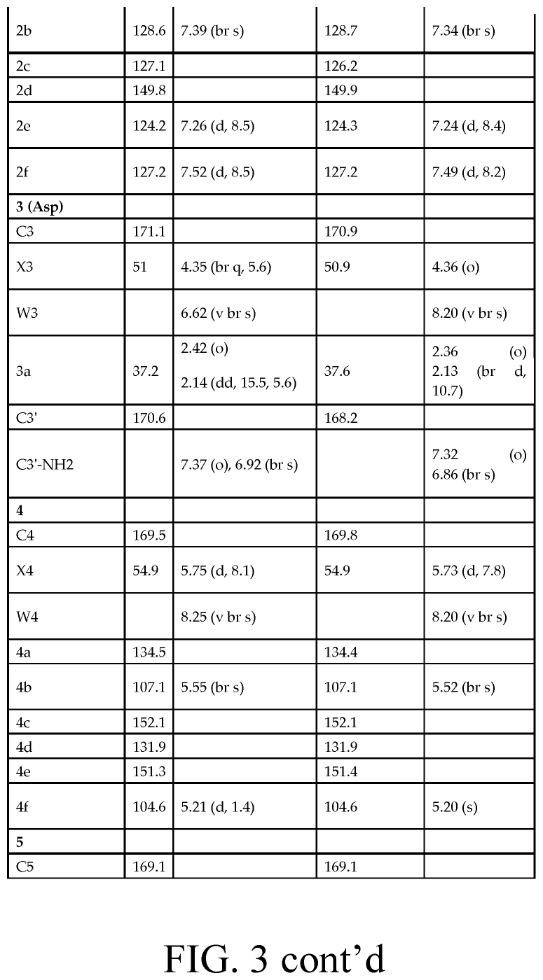

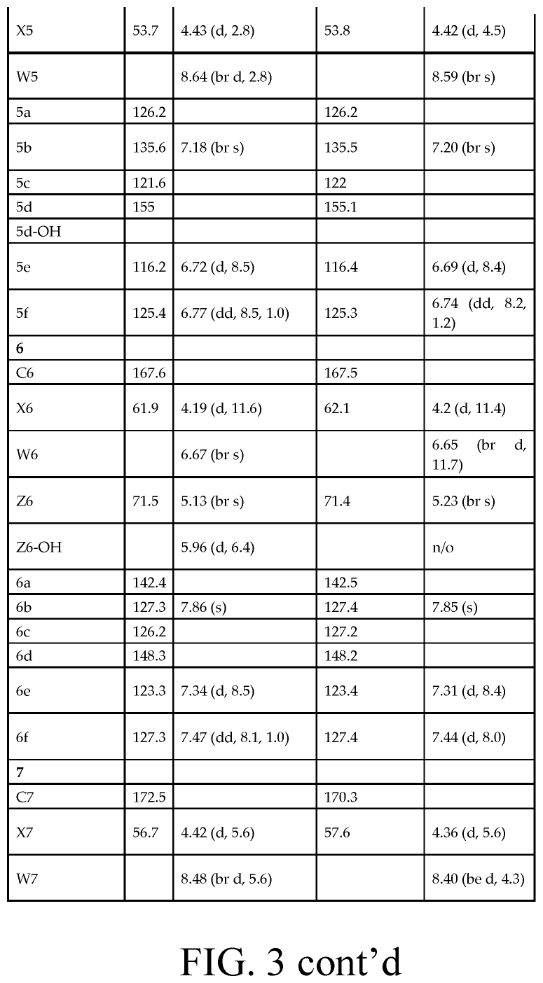

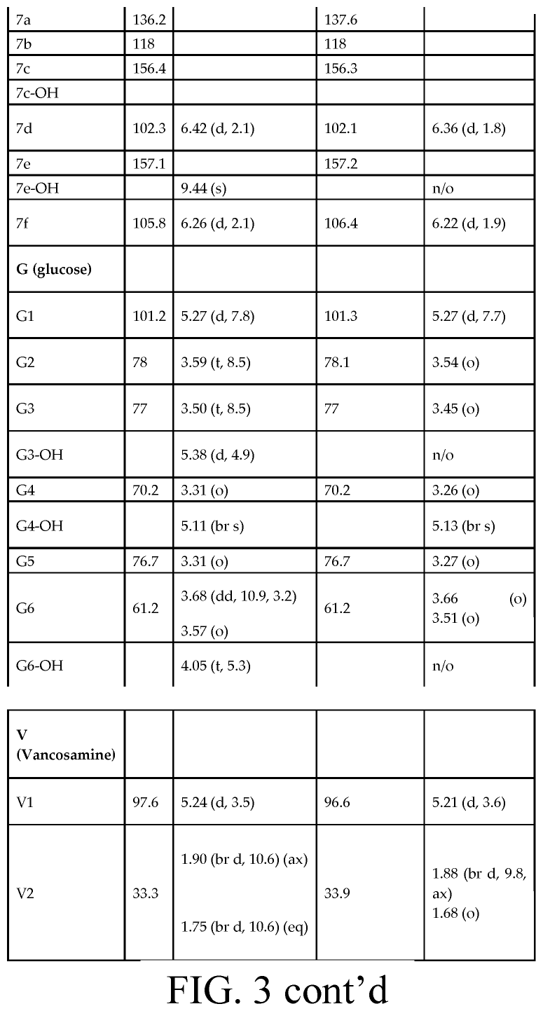

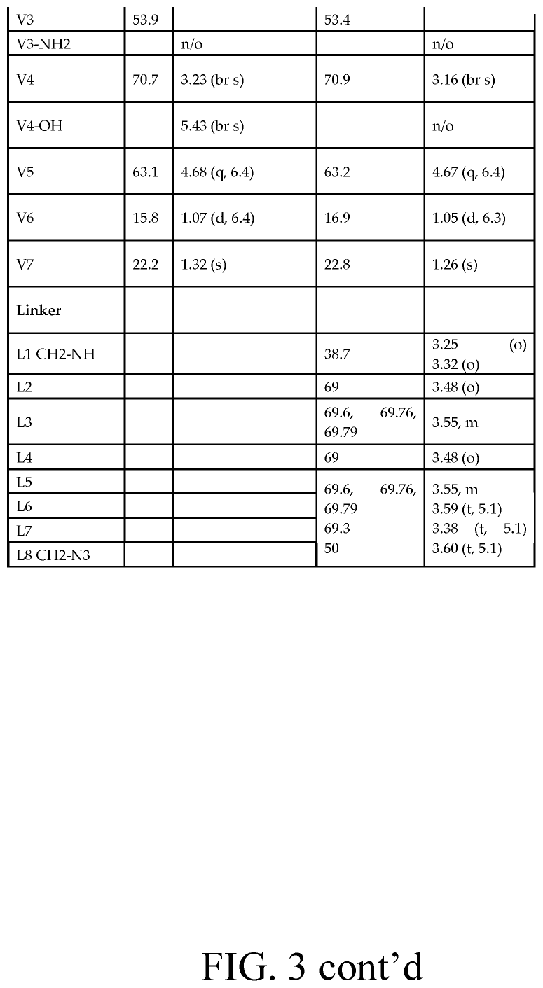

The compound was purified by preparative HPLC to give N₃-PEG3-Van (168 mg, 23% yield) as white solid. Preparative HPLC was run on an Agilent Technologies (1260 Infinity) instrument using a preparative column (Agilent Eclipse XDB-Phenyl, 30×100mm, 5 μm particle size) with a gradient elution (flow rate 20 mL/min, room temperature using 0.05% formic acid in water and 0.05% formic acid in acetonitrile as eluents, gradient timetable: 5 to 50% B for 20 min, wash). The resulting N₃-PEG3-Van was characterized by NMR and LCMS (FIGS. 3-4)

### Preparation of Separation Nanoparticles

For this example, carboxylated superparamagnetic 170 nm nanoparticles (EMD Millipore, M1-020/50) were used for the construct. A monolayer of human serum albumin (HSA) was bound onto the surface of the nanoparticles by carbodiimide carboxyl activation in order to mitigate nonspecific interactions when deployed in biological fluids (i.e. to passivate the nanoparticles).

Specifically, the superparamagnetic carboxylated nanoparticles were coated with human serum albumin (HSA) using carbodiimide EDC (EDAC) and Sulfo-NHS (N-hydroxysulfosuccinimide) reaction according to the manufacturer. The amount of HSA was determined according to [1]. Briefly, for 500 uL of carboxylated nanoparticles, 6.25 mg of both sulfo-NHS (Thermo Scientific) and carbodiimide EDC (Chem-Impex International Inc) were dissolved in ice cold MES (2-(N-Morpholino) ethanesulfonic acid sodium salt) buffer pH 5.0 (Sigma Aldrich). After 15 min, the MPs were washed quickly in PBSP (phosphate buffered saline, 0.1% pluronic F-127 (Sigma Aldrich)) buffer, then 50 mg human serum albumin (HSA) (Sigma Aldrich) dissolved in PBSP was added and incubated for 2 hours at room temperature with mixing. The nanoparticles were then washed and suspended in PBSP buffer.

The generation of an HSA layer on the nanoparticles was assessed by bicinchoninic acid assay (BCA) using a BCA kit (Thermo Scientific) and was quantified with a dilution series of a standard HSA protein (approximately 2×10⁶ HSA molecules/μm²).

### Coupling of Second Linker to the Separation Nanoparticles

HSA molecules on the surface of the passivated magnetic nanoparticles were used to couple a second linker precursor in the form of N-hydroxy succinimide (NHS)-activated dibenzocyclooctyne (DBCO) hydrophilic PEG to the nanoparticles of the construct. Specifically, passivated HSA coated nanoparticles were coupled with the (NHS)-activated NHS-PEG4-NH—CO—CH₂—CH₂—CO-DBCO (Click Chemistry tools) by reacting 500 μL HSA-MPs (50 mg/mL) with 12.5 μL of NHS-PEG4-NH—CO—CH₂—CH₂—CO-DBCO linker (100 mg/mL) dissolved in DMSO for 2 hours at room temperature and then washed. Magnetic nanoparticles coupled to the second linker are hereinafter referred to as DBCO-PEG4-nanoparticles.

The DBCO layer was quantified by reacting 5 μL of DBCO-PEG4-nanoparticles with 1 μL N₃-NBD fluorescent dye (0.1 mg/mL, in-house) in 1% DMSO via copper free click chemistry through incubation in phosphate buffer for 1 hour at 37° C. The fluorophore-coupled nanoparticles were then washed 3 times with PBSP and resuspended in 100 μL as a final volume, with fluorescence measured on a Tecan M 1000 Pro plate reader at excitation of 460 and emission of 540 nm, using a negative control of unconjugated nanoparticles.

Coupling of N3-PEG3-Van with DBCO-PEG4-Nanoparticles

To form the final construct (also referred to herein as Van-NPs), N₃-PEG3-Van was linked to DBCO-PEG4-nanoparticles (See, FIG. 1). The azide group of N₃-PEG3-Van was reacted with the DBCO (alkyne) group of the DBCO-PEG4-nanoparticles such that the two components were connected via formation of a triazole group from cycloaddition of the azide and alkyne groups. Specifcally, washed magnetic nanoparticles coupled with NHS-PEG4-DBCO (i.e. DBCO-PEG4-nanoparticles) were conjugated to different concentrations of N₃-PEG3-Van (final conc. 0.125-3 mg/mL) in PBSP via copper free click chemistry by incubating them together for 4 hours at 37° C. As such, the magnetic nanoparticles were connected to vancomycin via the first linker and the second linker, as hereindescribed.

It will be appreciated that DBCO-PEG4-nanoparticles were expected to contain multiple NHS-PEG4-DBCO-containing second linkers attached to the surface thereof. It will be further appreciated that multiple individual N₃-PEG3-Van first linkers were expected to bind to the DBCO-PEG4-nanoparticles, via respective second linkers, such that the final bioseparation construct typically comprised a single nanoparticle coupled to multiple vancomycin molecules.

For this example, bioseparation constructs were produced with three different local densities of vancomycin molecules: high; intermediate; and low density. As hereinabove described, in this context “local density” is defined as the number of functionally active molecules per unit surface area (e.g. vancomycin molecules/μm²). For this example, surface area per bead was ˜0.1 μm², assuming that the beads are spherical with negligible surface roughness).

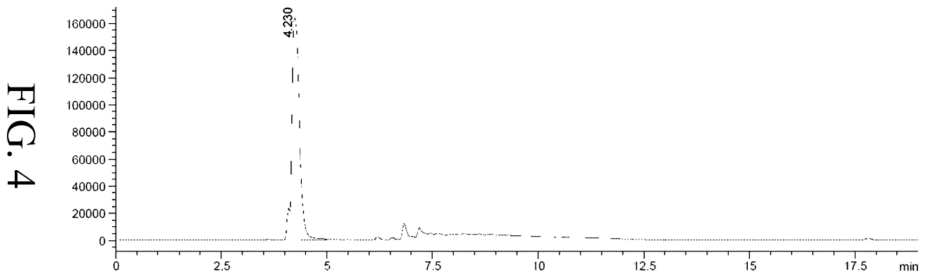

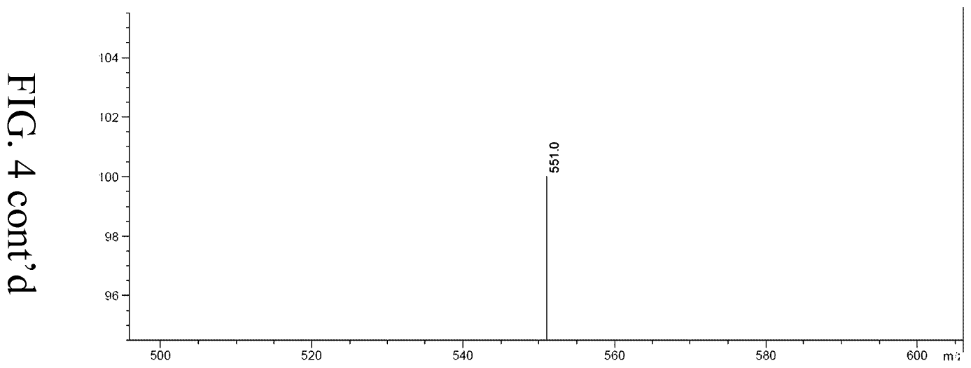

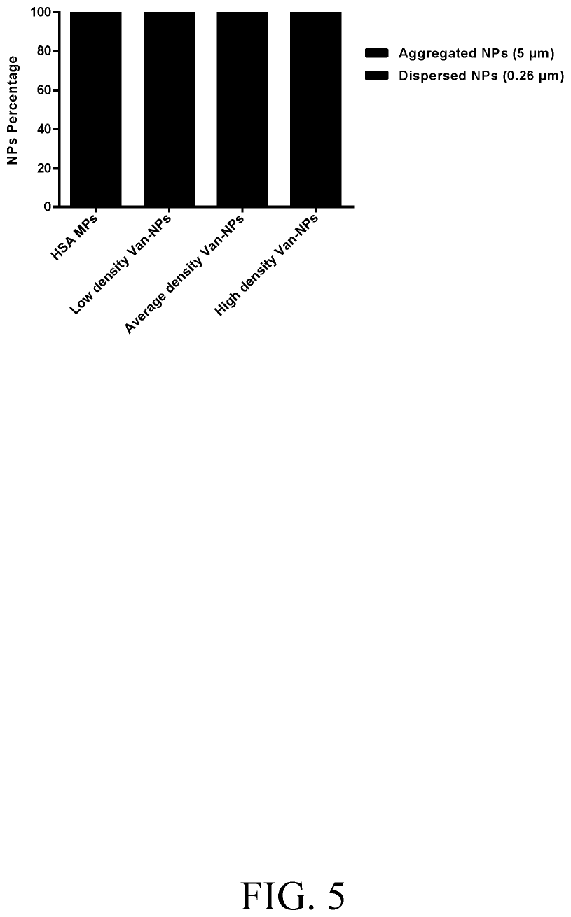

To vary local densities, the N₃-PEG3-Van was varied in different batches, while keeping the amount of DBCO-PEG4-nanoparticles constant. It will be appreciated that density could also be altered by varying the amount of NHS-PEG4-DBCO initially reacted with the nanoparticle. During these experiments, reversible nanoparticle aggregation was observed at high vancomycin loading concentrations, with aggregation easily dissipated by exposure to ultrasound. Dynamic light scattering (DLS) was used to monitor the evolution of the hydrodynamic volume for progressively increasing vancomycin loadings and found a corresponding increase in mean particle size (FIG. 5). It is postulated that both of these observations may be due to the tendency of vancomycin to form intra- and inter-nanoparticle dimers at high concentrations (˜745 mol/dm³).

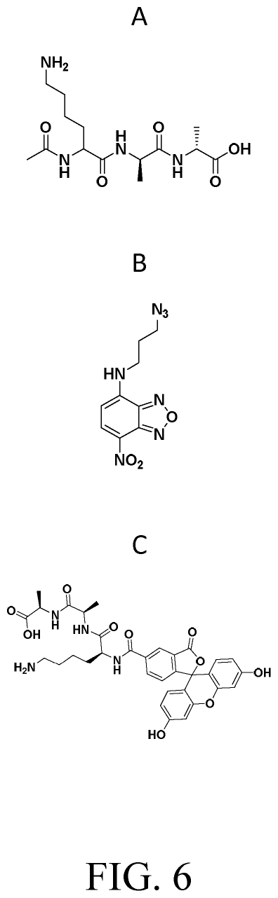

To assess local density, binding efficiency of N₃-PEG3-Van to DBCO-PEG4-nanoparticles was measured indirectly by quantifying the amount of N₃-NBD binding to unreacted DBCO molecules (i.e. DBCO-PEG4-nanoparticles) (FIG. 6). Additionally, active conjugated vancomycin was functionally quantified using two methods: 1) a fluorescent measurement of a fluorophore (Carboxyfluorescein, Fam) attached to a vancomycin-binding ligand, Fam-Lys-D-Ala-D-Ala (Fam-Kaa; (Custom synthesized by Mimotopes, 100 μg/mL in PBS) and 2) LCMS analysis of eluted Ac-Kaa after incubation with the bioseparation constructs (FIG. 6). Ac-Kaa (N-Acetyl-L-Lys-D-Ala-D-Ala) is a synthetic vancomycin target ligand that mimics the peptidic component of Lipid II involved in glycopeptide binding.

For the fluorescent indirect quantification of vancomycin coupling efficiency, 1 μL of bioseparation construct (nanoparticle concentration=25 mg/mL) was incubated with 200 μL Fam-Kaa (100 μg/mL) for 1 hour at 37° C. with rotation. Then, the nanoparticles were washed three times with PBSP and resuspended in 100 μL PBSP as a final volume, with fluorescence measured on a Tecan M 1000 Pro plate reader at excitation of 500 and emission of 520 nm. This allowed for quantification of the amount of vancomycin coupled DBCO molecules, with subtraction from the initial measurement of unreacted DBCO molecules providing an estimation of the amount of DBCO molecules/μm².

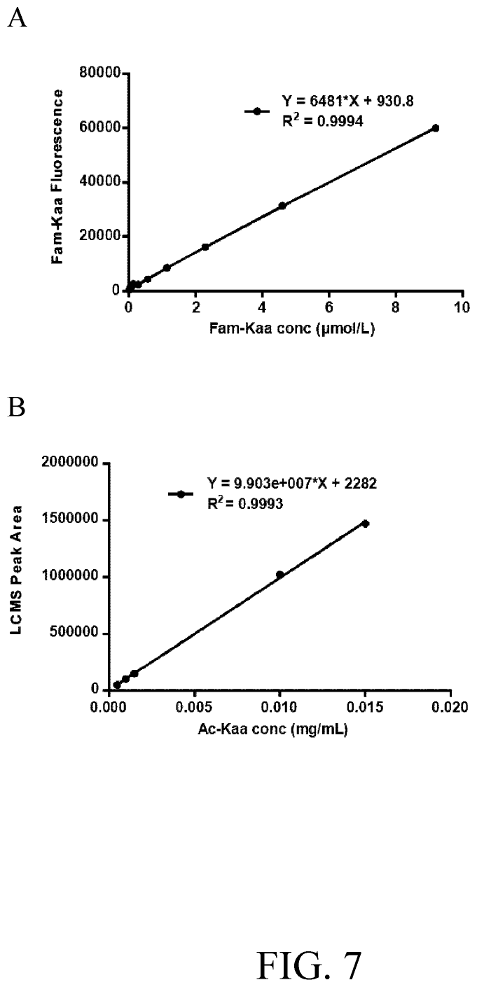

For the fluorescent Fam-Kaa direct determination, similarly, a constant amount from all Van-NP batches was incubated with Fam-Kaa. Unbound dye was removed by multiple washing steps, and then comparison of the fluorescence intensity of the washed nanoparticles with a reference curve enabled estimation of the surface density of active vancomycin covalently bound onto the nanoparticles (FIG. 7).

For the LCMS analysis of eluted Ac-Kaa, 25 μL of the prepared bioseparation construct batches (nanoparticle concentration 50 mg/mL) were also incubated with 25 μL of an excess concentration of Ac-Kaa (10 mg/mL) for 1 hour at 37° C. with rotation. Then, the nanoparticles were washed 3 times with PBSP and resuspended in 50 μL of 100 mM HCL and incubated for 15 min at room temperature. Then, the supernatant was diluted 1:1 with PBSP and the concentration of Ac-Kaa was measured on LCMS. (FIG. 7).

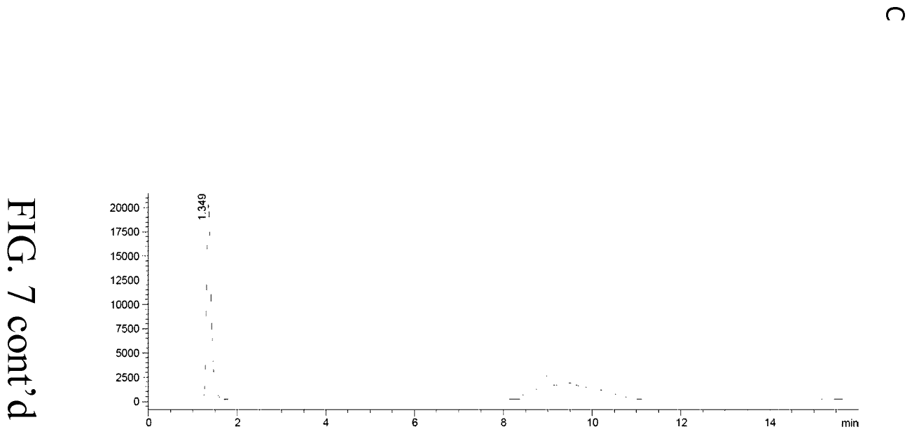

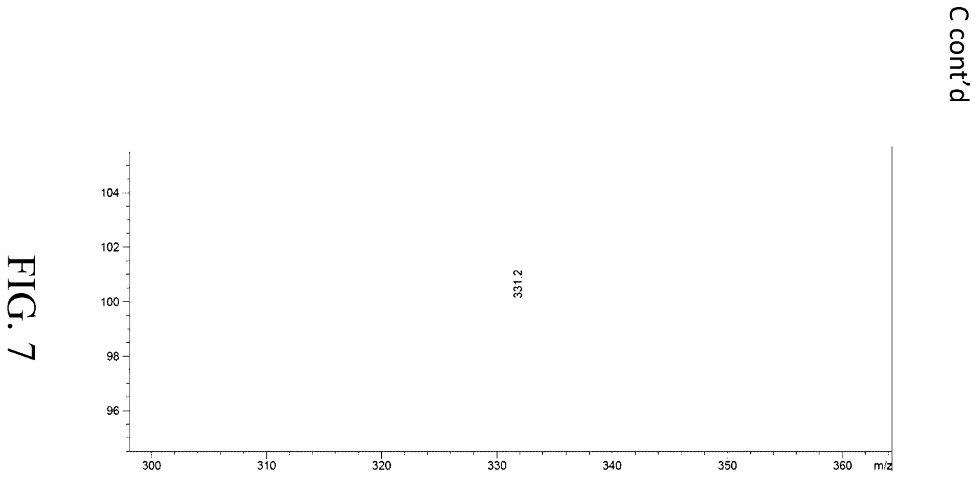

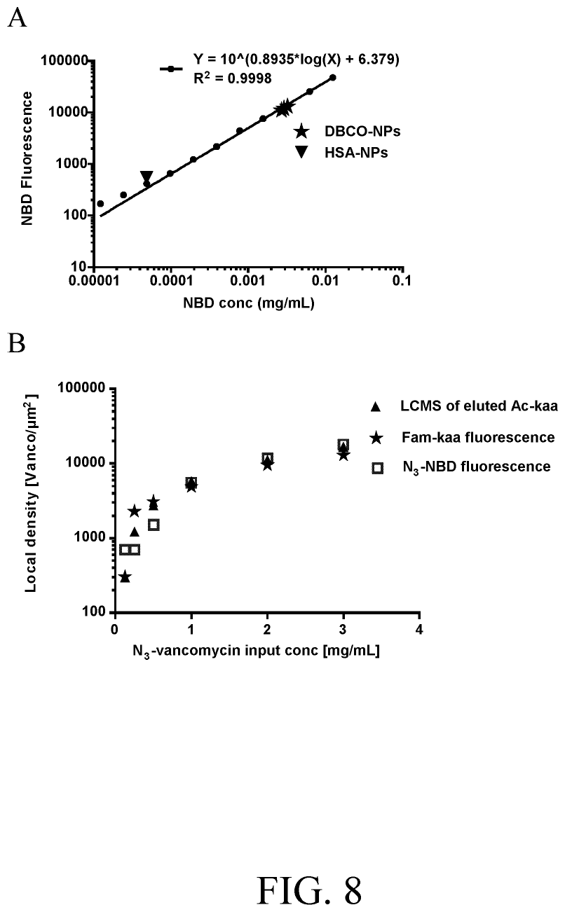

Vancomycin local density quantification by all three methods gave similar results (FIG. 8) though the Fam-Kaa and N₃-NBD fluorescent based assays were more sensitive, requiring 50- and 10-times less NPs than the LCMS method, respectively. For further discussion, local density based on the Fam-Kaa measurement is referred to.

### Example 2. Bioseparation and Bacterial Identification Using Constructs

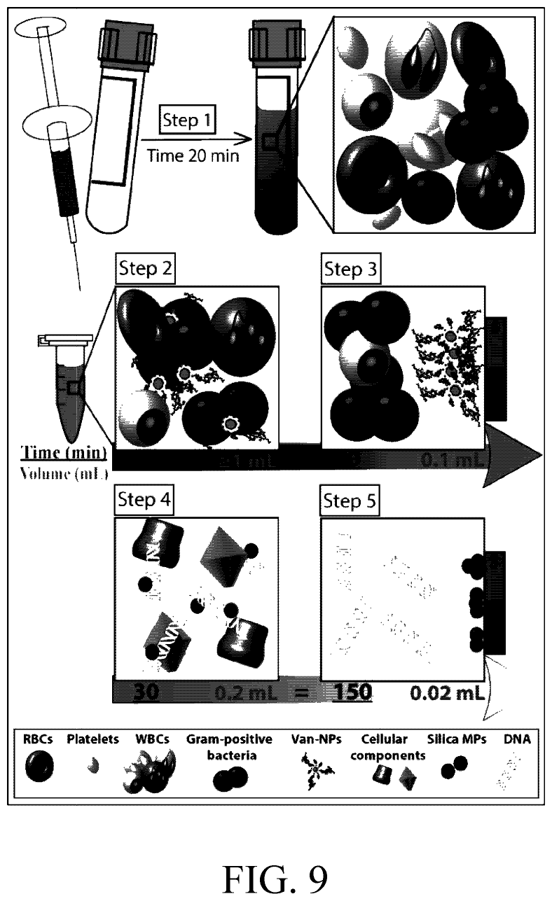

A schematic of a strategy used for bio separation and bacterial identification from samples using constructs of the invention is provided in FIG. 9. The strategy includes the steps of:

(1) Sample collection and preparation; and

(2) Bacterial magnetic capture using bio separation constructs of the invention, with subsequent washing and removal of interfering cells.

In regard to the removal of interfering cells, as described below, in relation to blood samples, the majority of interfering cells (red and white blood cells) are removed during step (1), which includes an aggregation step. In step (2), a small amount of remaining interfering human cells are then removed.

After step 2, optionally, step (3) Bacterial elution, may be performed.

After steps (2) or (3), bacteria can be identified using any suitable strategy, as hereinabove described.

For applications wherein bacterial DNA or other cellular contents is analysed, such as for nucleotide sequence of the bacteria captured using the bioseparation construct, as depicted in FIG. 9 the strategy may further include steps 4 and/or 5;

(4) Bacterial lysis and extraction of cellular contents;

(5) Purification of cellular contents.

Details of representative methods and results for bio separation of Gram positive bacteria using bioseparation constructs of the invention are provided in this Example.

### Bacterial Strains

Bioseparation of the following Gram positive bacterial strains was assessed: Staphylococcus aureus sensitive (strain ATCC 25923); S. aureus MRSA (strain ATCC 43300); S. aureus GISA (strain ATCC 700698); S. aureus VRSA (strain NARSA VRS4); Staphylococcus epidermis sensitive (strain ATCC 12228); S. epidermis GISE (strain NARSA NRS60); Streptococcus pneumoniae sensitive (strain ATCC 33400); S. pneumoniae resistant (strain ATCC 700677).

The bacterial strains were cultured on nutrient broth at 37° C. and diluted to the required concentration based on OD600 calculations. It was assumed that OD600=0.5 corresponds to 10⁹ cfu/mL. To prepare samples for assessment of bioseparation using constructs of the invention, the sample was spiked with a defined amount of bacteria depending on the experiment being conducted. A subsample of the bacterial cells (approximately 100 CFUs) was cultured on nutrient agar to validate the level of bacterial loading (input).

Bioseparation of Bacteria from Platelet Samples

The efficiency of bioseparation of bacteria (alternatively referred to herein as ‘bacterial capture’ or ‘bacterial cell capture’) using the construct as described in Example 1 was assessed in platelet samples containing S. aureus sensitive and S. epidermidis sensitive.

2 μL of 170 nm bioseparation constructs was added to each bacteria-spiked platelet sample, and the sample was incubated for 1 hour at 37° C. with rotation/shaking to prevent sedimentation. Bacterial cells bound to the bioseparation constructs were extracted from the 1 mL sample volume by placing the tubes on a magnetic rack for 2-5 minutes. Then, bioseparation construct bound bacteria were washed magnetically with sterile PBSP (phosphate buffer saline, 0.1% pluronic F-127) 3 times. Bioseparation constructs were then resuspended in 100 μL of sterile PBS buffer. The bacteria obtained was then cultured on nutrient agar and incubated for 24 hr at 37° C.

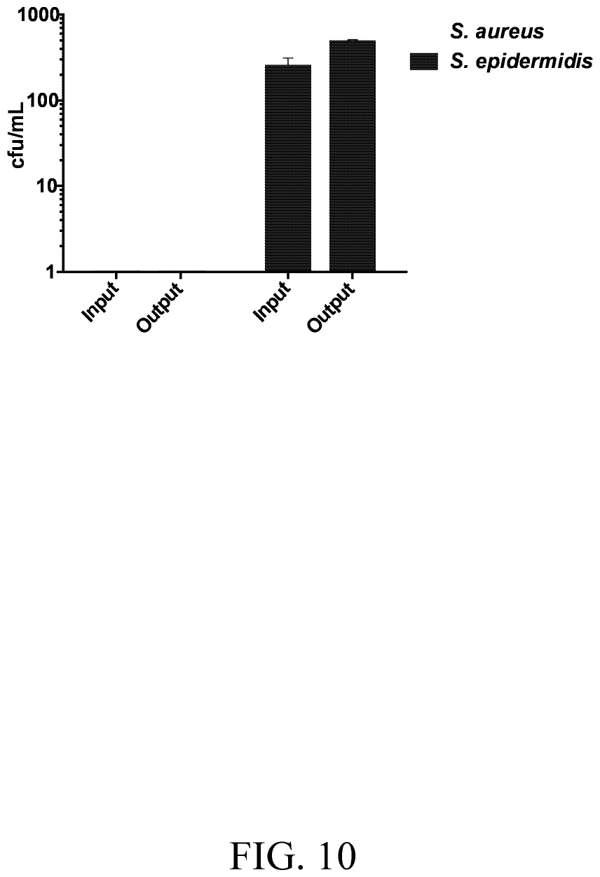

Bioseparation efficiency was calculated based on the calculated number of the counted colonies of the output plates in comparison to the input plates in cfu/mL. As set forth in FIG. 10, bioseparation efficiency was extremely high for both bacterial strains assessed.

Bioseparation of Bacteria from Blood Samples

The efficiency of bioseparation of bacteria using the construct as described in Example 1 was assessed in blood samples containing Staphylococcus aureus sensitive; S. aureus MRSA; S. aureus GISA; S. aureus VRSA; Staphylococcus epidermis sensitive; S. epidermis GISE; Streptococcus pneumoniae sensitive; and S. pneumoniae resistant.

To prepare blood samples for spiking with these bacteria, sterile whole blood samples were treated with 1:1 ratio of RBC aggregating solution (2% (w/v) Dextran-T500, 0.2% (w/v) D-glucose dissolved in SSP+ solution) and left to sediment at room temperature. Then, 1 mL of supernatant blood solution was transferred to a clean sterile Eppendorf tube and spiked as described above, with a subsample of cells cultured for validation of input.

To determine the extent of reduction in interfering blood cells due to aggregation using this approach, the effect of aggregation on the count of blood cellular content at different time points was performed using hemocytometer. An 80 and 98% decrease in total RBCs and WBCs count/mL at 10 and 20 min, respectively, was observed. Furthermore, reduction of interfering cells up to 99% cells/mL with longer incubation time (30-40 min). For the current example, aggregation time of 20 minutes was used. However, aaggregation time may be increased for larger sample volume (e.g. 10 or 20 mLs) up to 5 hours.

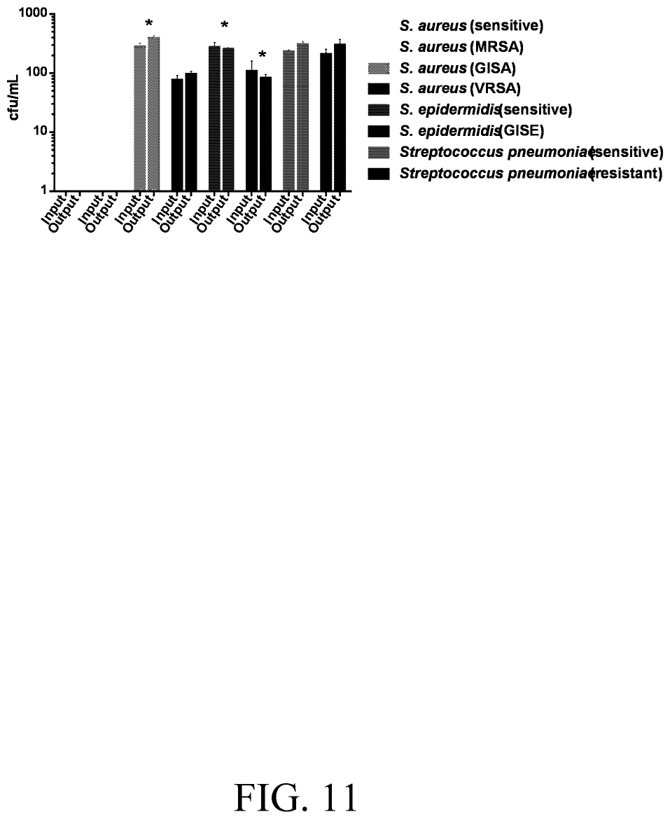

After aggregation, bioseparation constructs were then added to bacteria spiked samples and processing for capture was performed, as described above for platelet samples. As for the platelet samples, bioseparation efficiency was calculated based on the calculated number of the counted colonies of the output plates in comparison to the input plates in CFU/mL Similar as for the platelet samples, as set forth in FIG. 11, bioseparation efficiency was extremely high for all bacterial strains assessed.

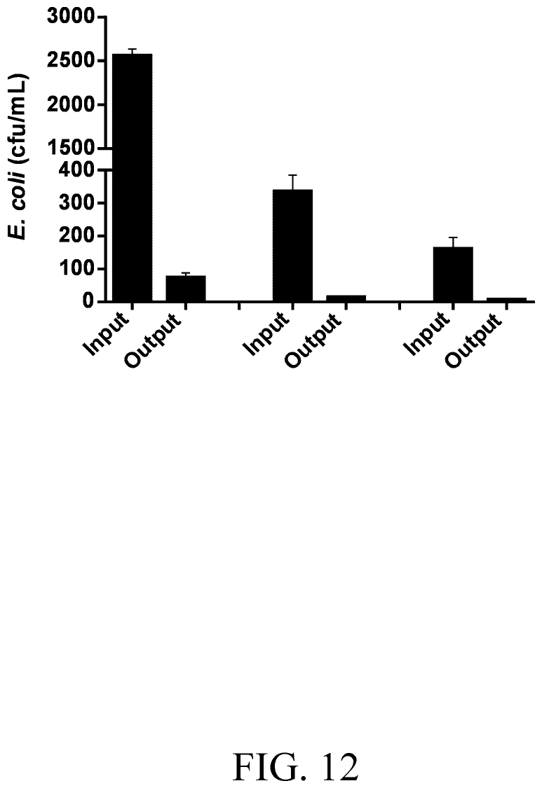

Additionally, to assess the specificity of bioseparation of Gram positive bacteria using the constructs, samples spiked with Escherichia coli (strain ATCC 25922), which is Gram negative, were subject to attempted bacterial capture as described above. As set forth in FIG. 12, non-specific capture of Gram negative E. coli was low.

### Bacterial Elution

Magnetically separated construct bound bacteria from the samples mentioned above were resuspended in 100 μL acetic acid (100 mM, pH 4) and incubated for 20 min at 65° C. The decrease in the pH of the solution resulted in elution of bacteria from the construct. After 20 min incubation, the Eppendorfs were kept on a magnetic rack to allow for magnetic separation, under the magnetic field, and the supernatant was transferred to new sterile Eppendorfs.

Replicates of magnetically captured bacterial cells were resuspended in sterile PBSP and sub-cultured on LB agar plates and incubated at 37° C. for 24 hours as a control of bacterial count.

Bacterial Lysis, DNA Capture and qPCR Detection

As described above, for some downstream applications (e.g. nucleotide sequencing) bacterial lysis and DNA capture is desirable. For this example, bacterial lysis was performed after capture as set forth above, and DNA from the lysed bacteria was subsequently obtained and purified.

Additionally, efficiency of DNA obtained from Gram positive bacteria in samples was compared between (A) bioseparation according to the process described herein, using capture by constructs of the invention and subsequent bacterial lysis and DNA purification; (B) DNA extraction using commercial a Qiagen ‘DNeasy Blood and Tissue’ kit with; and (C) DNA extraction using the Qiagen kit with PBS buffer.

For bacterial lysis and DNA extraction, eluted bacterial samples were further incubated for further 20 min at 65° C. Then, 100 μL of 13 M guanidine HCl containing solution (Final concentration: 6.5 M Gu HCl (Sigma Aldrich), 10 mM Tris HCl pH 8.0 (Invitrogen), 1 mM EDTA pH 8.0 (Invitrogen), 5 mM Tris base pH 10.7 (Astral Scientific)) was added. After mixing, 6 μL of magnetic silica particles (MagPrep® Silica HS, Merck) were added and mixed by vortex and incubated for 15 min at 37° C. with rotation. DNA bound magnetic silica were washed with 80% Isopropanol (Merck) 2 times magnetically. Then, 20 μL of 100 mM Tris HCl (pH 9.0) was added and allowed for DNA elution by incubating for 15 min at 65° C. The 20 μL eluted DNA was transferred to a sterile Eppendorf and stored in −20° C.

DNA extraction using the Qiagen kit (with or without PBS buffer) was performed according to the manufacturer's instructions.

To assess and compare the level of bacterial DNA captured according to the process described herein, and by use of the Qiagen kits, qPCR was conducted using primers specific for bacterial DNA. Additionally, the level of human DNA captured from the samples by the respective approaches was quantified.

Specifically, Sybr green (SYBR® Select Master Mix, Life Technology) was used in the master mix (1×) with 2 μM primer concentration and 1 μL of either genomic DNA (100 ng/μL, control) or test DNA samples obtained using the process described herein, or Qiagen kit with or without PBS buffer. qPCR cycles were as follows: 95° C. 10 min followed by 40 cycles of 95° C. 15 sec, 55° C. 20 sec and 72° C. 30 sec, then melting cycle of 95° C. 15 sec, 60° C. 1 min and 95° C. 15 sec.

All results were quantified to standard curves of amplified serial dilution of genomic DNA of S. aureus and human DNA. For detection of bacteria, the Gmk gene was amplified by the following primers (F: 5′-TCGTTTTATCAGGACCATCTGGAGTAGGTA-3′, and R: 5′-CATCTTTAATTAAAGCTTCAAACGCATCCC-3′) [2]. Human DNA was assessed by the same protocol but using the following primers: EFTUD2_GH_FP: GGTCTTGCCAGACACCAAAG, EFTUD2_GH_RP: TGAGAGGACACACGCAAAAC [3].

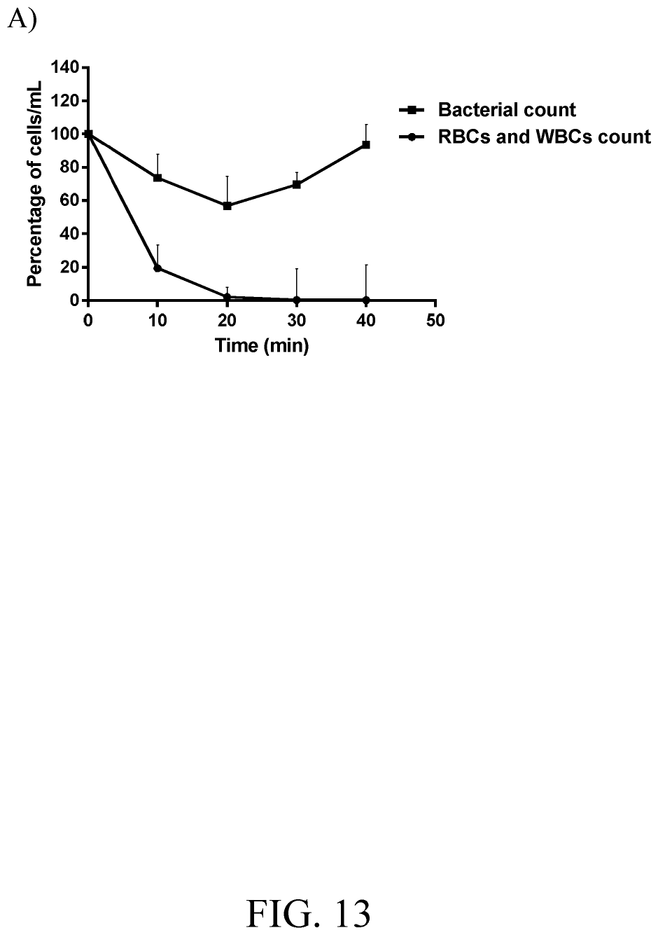

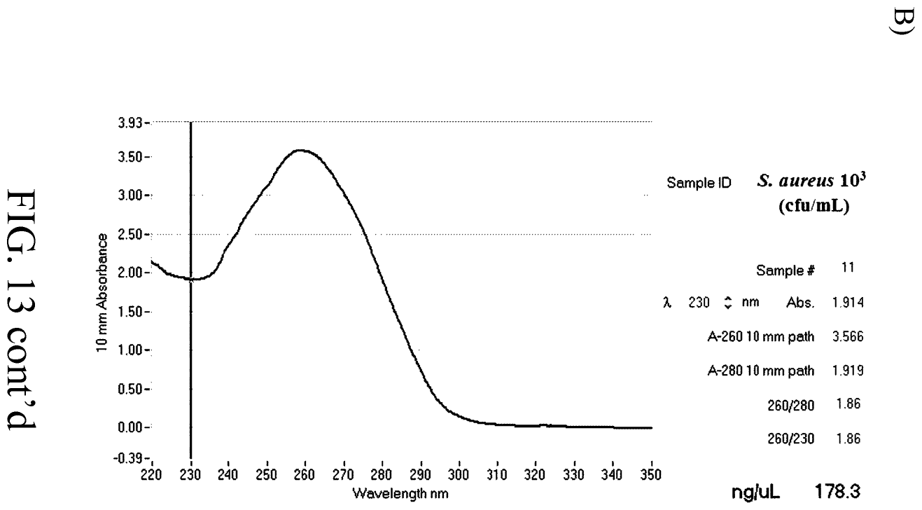

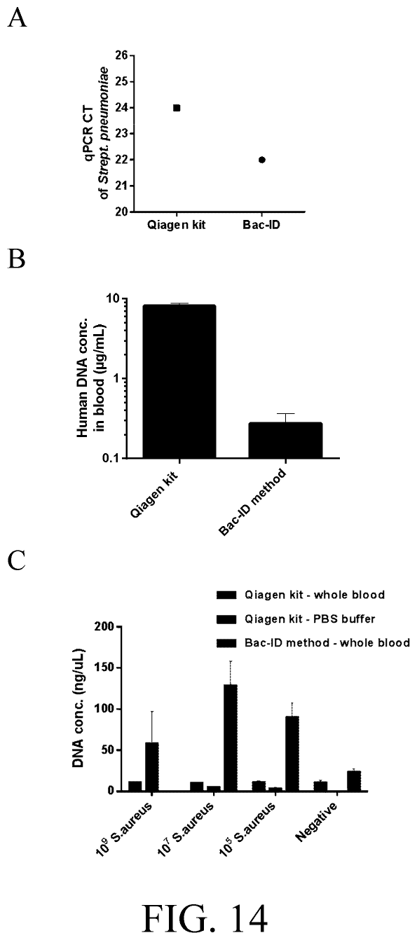

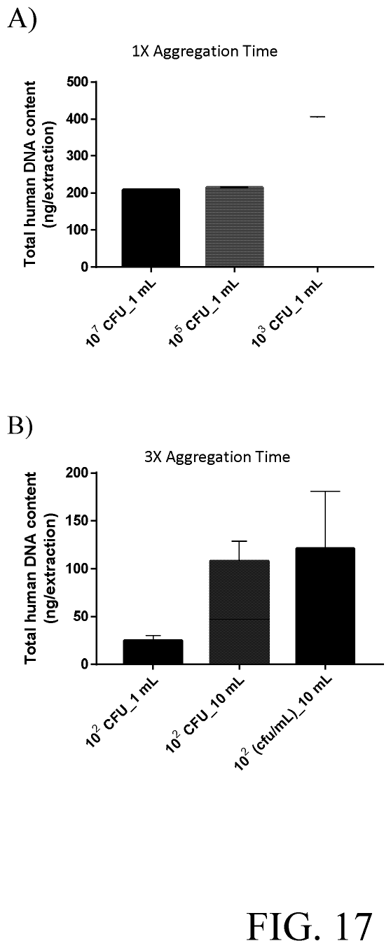

Results are set forth in FIGS. 13-17. As will be evident from FIG. 13, DNA of high purity and concentration was obtained by bioseparation and subsequent lysis and DNA processing (which is referred to alternatively in the Figures and this example as ‘Bac-ID’). As will be evident from FIGS. 14A and 14C, Bac-ID was typically more sensitive for obtaining Gram positive bacterial DNA from blood samples spiked with S. aureus, than extraction using Qiagen kits with or without buffer. Furthermore, as expected, Bac-ID yielded far lower concentrations of human DNA (FIG. 14B and FIG. 17). In FIG. 14C, Sample of 10⁹ S. aureus (cfu/mL) was only assessed by Qiagen kit to assess inhibition of the kit with blood samples. Samples with high concentration (10⁹ S. aureus cfu/mL) were not assessed by Bac-ID method in this example.

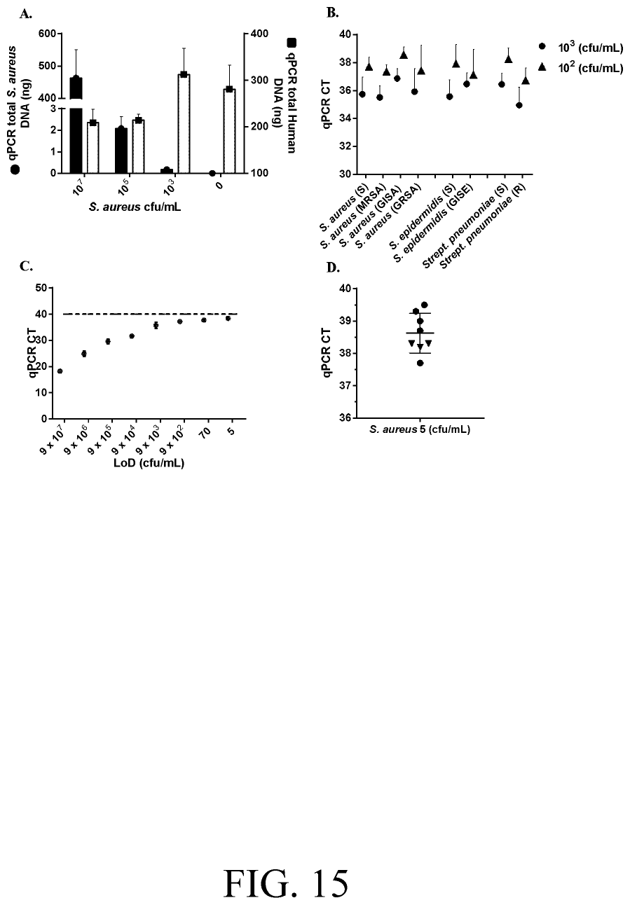

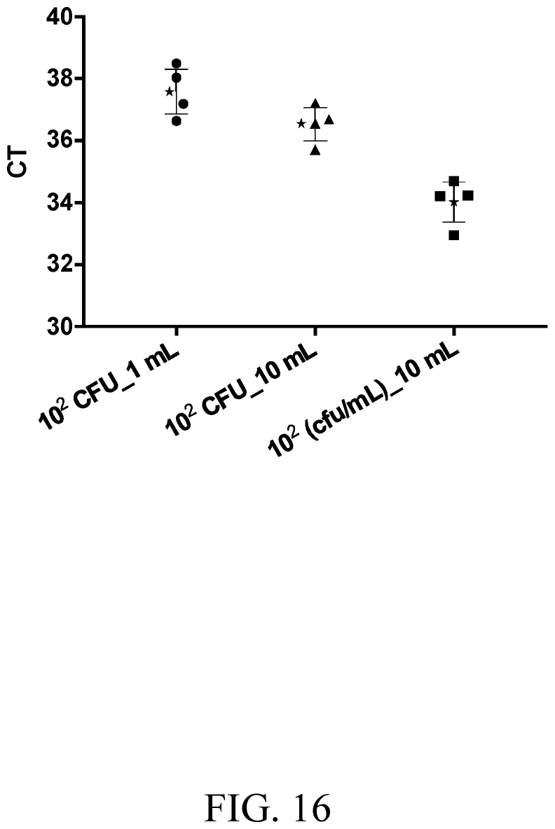

Additionally, as will be evident from FIG. 15B, Bac-ID effectively obtained Gram positive DNA from all species and strains assessed for this example (i.e. Staphylococcus aureus sensitive; S. aureus MRSA; S. aureus GISA; S. aureus VRSA; Staphylococcus epidermis sensitive; S. epidermis GISE; Streptococcus pneumoniae sensitive; and S. pneumoniae resistant). Additionally, the amount of DNA detected correlated with the amount and concentration of bacteria spiked into the samples (FIGS. 15C and 16). Furthermore, repeatability of the Bac-ID method with respect to the amount of bacterial DNA obtained was high (FIG. 15D). FIG. 16 demonstrates the applicability of the method to larger sample volumes with the same extraction efficiency and FIG. 17 demonstrates that increasing the aggregation time decreases the human DNA content which may be required in some applications.

### Example 3. Antimicrobial and Binding Activity of Constructs

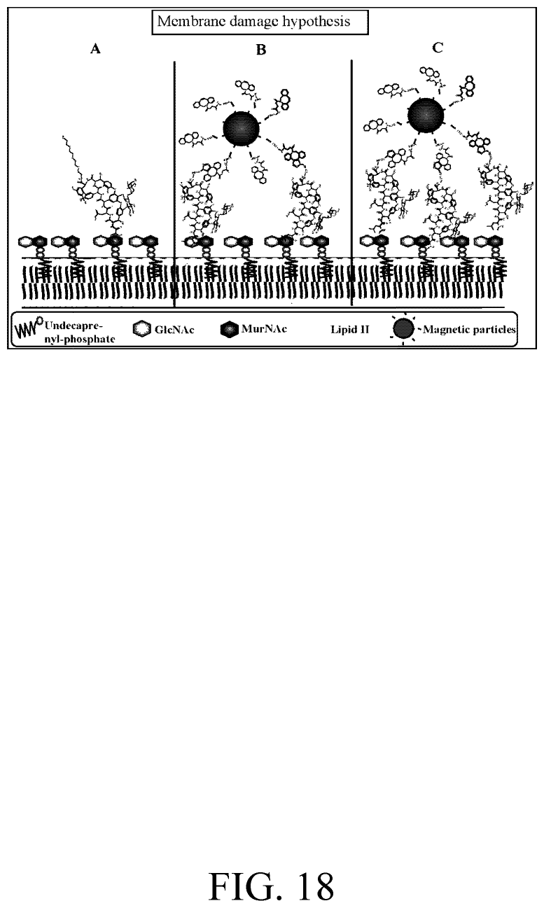

The constructs produced as described in Example 1 were assessed for Minimum Inhibitory Concentration (MIC), binding avidity, and bacterial membrane permeability, against Vancomycin sensitive and resistant S. aureus and Vancomycin-resistant Enterococcus (VRE). As described in detail below, it was surprisingly found that intermediate and high local density constructs showed substantially lower MIC against both sensitive and resistant strains, and also showed higher binding avidity and bacterial membrane permeability. It is hypothesized that intermediate and high density Van-NPs allow for multiple more strongly localized binding events on the bacterial cell wall whereas when the drug is in solution Brownian diffusion randomizes the binding events over a wider surface area. Accordingly, a high drug density on nano-carriers caused rapid and localized membrane damage (FIG. 18).

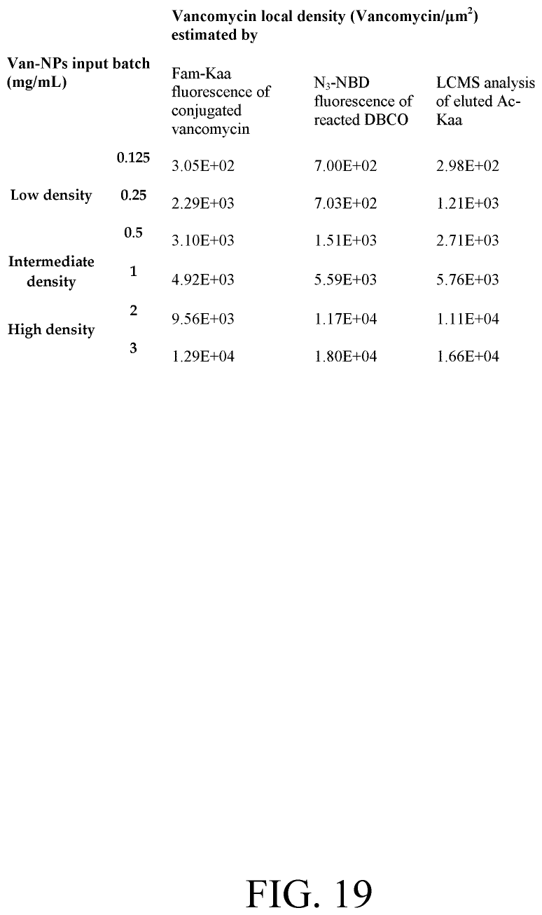

In this example, the minimum inhibitory effect (MIC) of low, intermediate, and high vancomycin local density constructs (FIG. 19) was assessed against vancomycin sensitive and resistant strains, with the vancomycin content for MIC determinations calculated based on the Fam-Kaa assay as described in Example 1. Vancomycin content varied from 0.5-30 μg/mL. HSA-coated magnetic nanoparticles (HSA-nanoparticles) as described in Example 1 were used in MIC studies as a positive control to confirm that the coated nanoparticles themselves did not possess any antimicrobial activity.

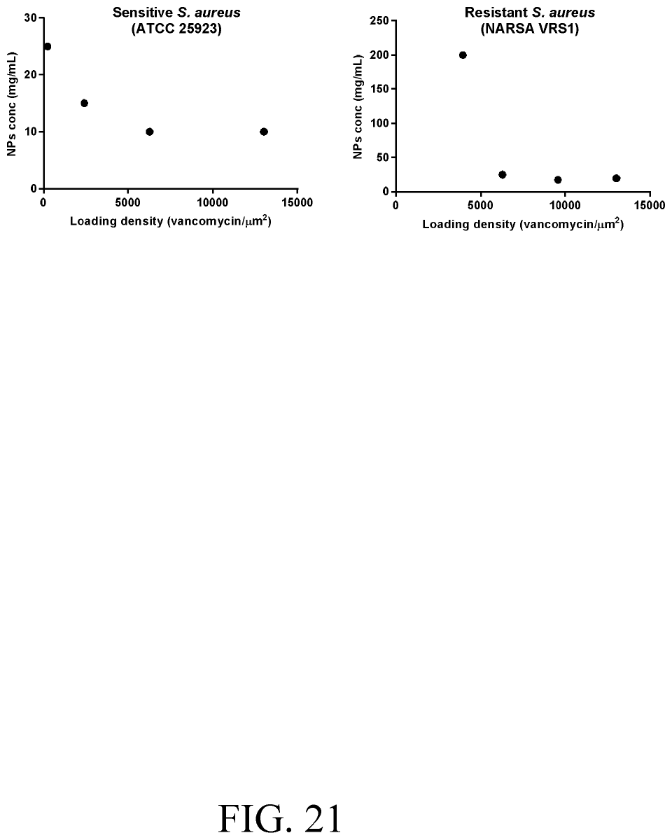

At intermediate and high vancomycin local density, inhibition of bacterial growth by the constructs occurred against resistant and sensitive strains, respectively, while turbidity was observed with HSA-nanoparticles due to microbial growth. Notably, we observed 14- to >100- and 2- to 18-fold improvements in MIC with the constructs compared to N₃-PEG3-Van and unmodified vancomycin, respectively, when tested against sensitive and resistant S. aureus and VanA and VanB resistant Enterococcus strains (FIGS. 20 and 21). This significant enhancement of vancomycin potency showed that bacterial inhibition was affected by the local density, with even greater improvements in activity compared to free vancomycin against the strains.

To further explore this phenomenon, the concentration of nanoparticles within the constructs was titrated (200-5 mg/mL) for a given local density of vancomycin, and the concentration of constructs required to achieve inhibition of bacterial growth for a given local density of vancomycin was calculated. The results showed an indirect relationship between vancomycin local density and the number of constructs required for inhibition: higher densities required fewer constructs, for both vancomycin-sensitive and resistant strains (FIG. 21).

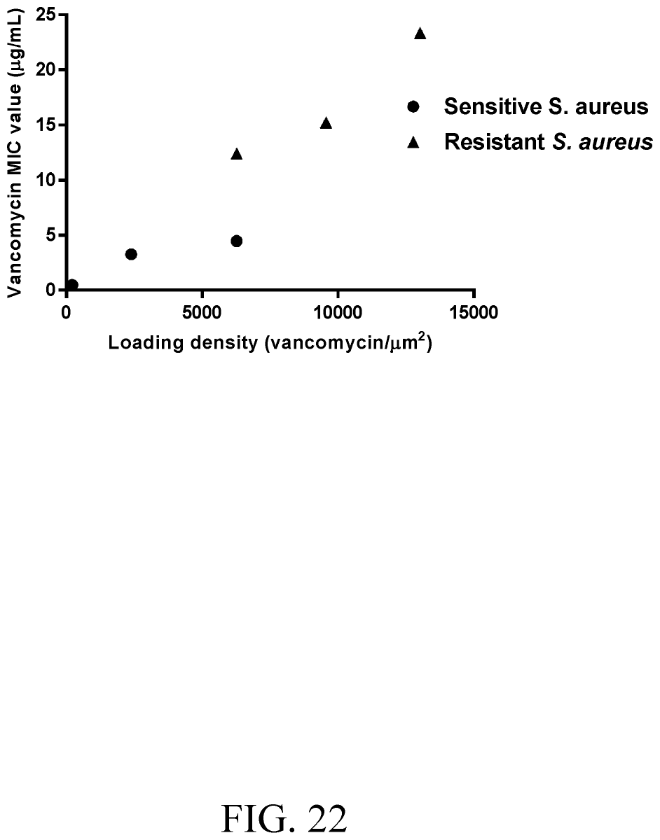

When MIC values for each strain were calculated based on the conjugated vancomycin content (based on the vancomycin loading and the number of constructs required for inhibition), the MICs were found to vary with increasing local density (FIG. 22 and Table 1). For the vancomycin sensitive S. aureus strain (ATCC 25923), an MIC of 0.55 μg/mL was detected with a low density (2.18×10² vancomycin/μm²) but high number (25 mg/mL nanoparticles) of constructs. Increasing the local density to 2.41×10³ or 3.94×10³ vancomycin/μm² required a reduced number of constructs (15 and 12.5 mg/mL, respectively) resulted in MIC values corresponding to 3.3 and 4.5 μg/mL vancomycin, respectively. For a resistant S. aureus strain (NARSA VRS-1), an MIC of 12.4 μg/mL vancomycin was obtained with intermediate density Van-NPs (6.28×10³ vancomycin/μm² at 25 mg/mL nanoparticles) while a higher MIC of 15.2 μg/mL was obtained with a higher local density (9.56×10³ vancomycin/μm² at 17.5 mg/mL nanoparticles).

In contrast, low vancomycin density constructs were not able inhibit the growth of the resistant strain, even with very high numbers of nanoparticles. The results demonstrate that the MIC value is a result of both the local density of vancomycin on the surface of nanoparticle and the number of nanoparticles (and therefore the number of constructs), which together represents the number of binding events occurring on the bacterial membrane for efficient killing. Depending on the resistance profile of the strain a specific local density is required, with a higher the local density required with highly resistant strains. However, for a given strain, the lowest possible local density gives the lowest MIC value (based on calculated conjugated vancomycin content).

To elucidate the fundamental mechanism behind enhanced drug potency of vancomycin constructs as compared to free vancomycin, the binding affinity of vancomycin, N₃-PEG3-Van and the constructs (Van-NPs) to the bacterial ligand target by using a ligand displacement assay was tested. This allowed assessment of what concentration of competing Ac-Kaa ligand was required to abrogate antibacterial activity. The test compounds were assessed at 10-fold MIC in the presence of a wide range of Ac-Kaa molar excess. The displacement by Ac-Kaa is used to demonstrate the differences in affinities to the native bacterial Kaa target of Lipid II [4]-[5]. The design of the experiment is based on calculating the Ac-Kaa concentration required for the vancomycin molecules to favor binding to Ac-Kaa instead of inhibiting bacterial growth: the higher the Ac-Kaa concentration required, the higher the vancomycin bacterial binding affinity.

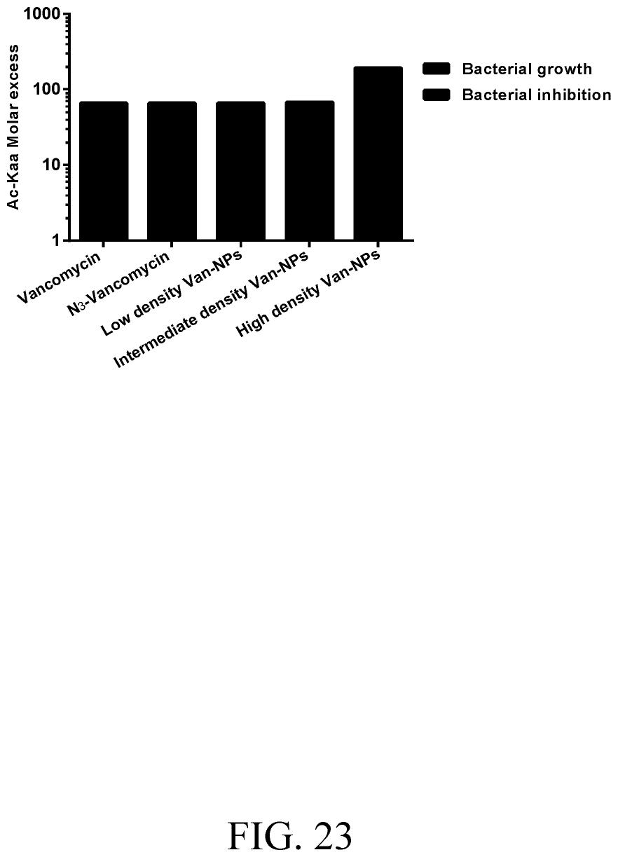

The results showed that vancomycin, N₃-PEG3-Van and low vancomycin density constructs had the same affinity to the bacterial ligand, with >2-fold molar excess of Ac-Kaa (based on vancomycin molecular weight) resulting in bacterial growth (FIG. 23). However, intermediate and high vancomycin density constructs showed a stronger affinity to bind to bacteria, requiring >4 and 64 molar excess of Ac-Kaa, respectively to overcome bacterial inhibition (FIG. 23). These findings demonstrate that there is a direct relationship between increasing the local density of glycopeptide antibiotic constructs and bacterial binding affinity.

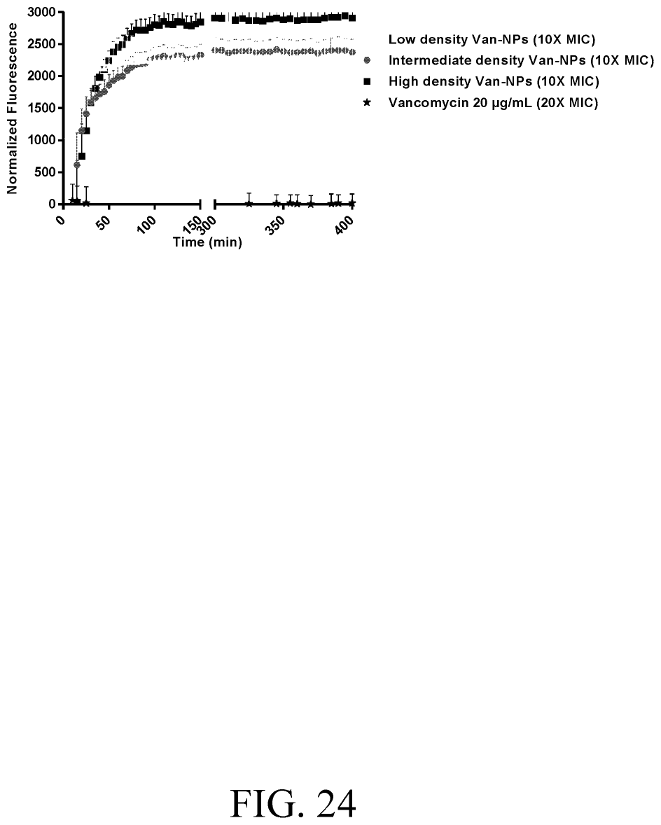

To determine the extent of bacterial cell wall damage, we incubated S. aureus with vancomycin (at 20-fold MIC) and constructs of different local densities (at 10-fold MIC) in the presence of propidium iodide, and measured the fluorescence over time. Propidium iodide (PI) is a fluorescent dye that assesses bacterial viability and membrane integrity through increase of its fluorescence when bound to the bacterial nucleic acid content [6]. Remarkably, vancomycin did not show any membrane damage after 10 hours, while all local densities of constructs showed membrane permeabilization of PI after 1 hour incubation (FIG. 24).

The high local density constructs showed higher fluorescence signal (2800) compared to low and intermediate density Van-NPs (2200), indicating that higher localized drug induced considerably more membrane leakage. Surprisingly, the low local density constructs triggered earlier membrane damage (increased fluorescence seen at the first 5 min compared to 20-25 min with other Van-NPs densities) which may be due to the higher number of NPs (NPs conc.: 25 mg/mL) compared to intermediate and high density Van-NPs (NPs conc.: 10 mg/mL) leading to an increased binding events and binding kinetics rate.

These results support the membrane damage hypothesis of high local density construct, where multiple vancomycin molecules cause higher binding avidity that lead to a localized and severe membrane damage in a concentrated surface area at the same time. This increased membrane damage of high density Van-NPs likely increases the construct affinity against thick cell walled-resistant bacteria and compensates for the modification of the Lipid II target ligand in the VanA and VanB resistant strains.

In summary, this example surprisingly revealed that controlled conjugation of antibiotics onto nanoparticles in constructs as described in Example 1 resulted in enhanced antibiotic efficacy with improved MIC (based on conjugated antibiotic content) against both sensitive and resistant strains, with optimum local density depending on the bacterial strain. The constructs showed increased affinity to the bacterial binding targets, and the high local density constructs induced significant bacterial membrane damage in 2 hours, compared to no damage by the free antibiotic after 10 hours. As a consequence, there is a new hope to target superbugs using current antibiotics by designing nanotechnologies to immobilize readily available antibiotics on biocompatible nano-carriers. This finding is of potential impact to tackle drug resistance and may prevent the development of new resistance mechanisms.

### Example 4. Compounds Comprising Glycopeptide Antibiotics and Linkers

Certain compounds comprising vancomycin and linkers have been designed, and are considered particularly beneficial for use to produce constructs as described herein.

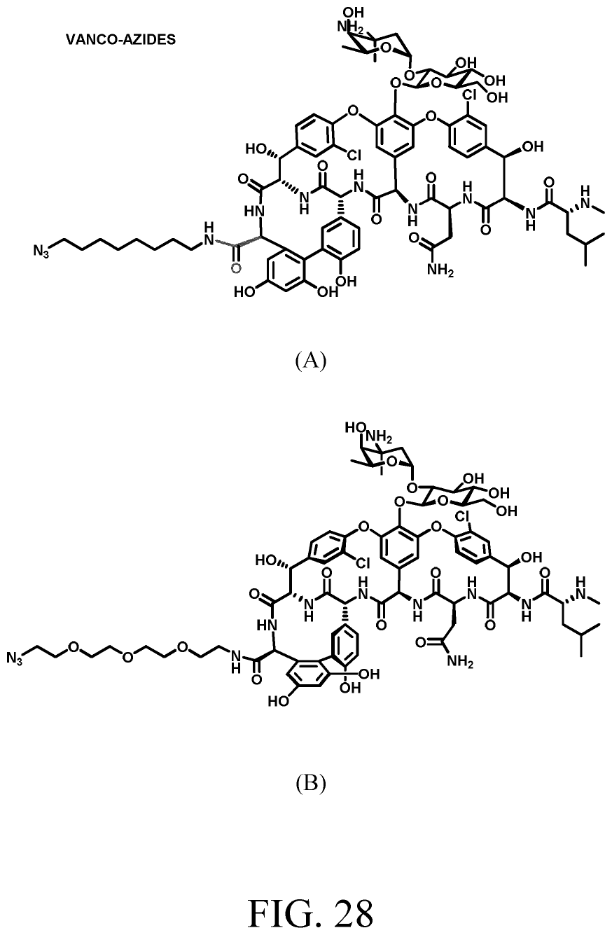

One such compound is N₃-PEG3-Van, as described in Example 1. The structure of N₃-PEG3-Van is set forth in FIG. 28B.

Another such compound is vancomycin combined with a linker in the form of a linear C8 chain with an amine moiety at a first end of the C8 chain and an azide moiety at a second end opposite the first end, wherein the amine moiety is directly linked to vancomycin by the C-terminal carboxy moiety (FIG. 28A). This compound is herein referred to as N₃-C8-Van.

### Example 5. Fluorescent Bacterial Identification Using Constructs

In this example, fluorescent carboxylated nanoparticles (0.2 μm blue fluorescent particles (365/415), Life technology) were coated with HSA as previously described. Fluorescent NPs were then coupled to NHS-PEG4-azide linker (Click Chemistry tools) by reacting 500 μL HSA-MPs (50 mg/mL) with 12.5 μL linker (100 mg/mL) dissolved in DMSO for 2 hours at room temperature and then washed. Anti-Staphylococcus aureus antibody (Thermo Fisher) was buffer exchanged to PBS using Zeba spin desalting column (MWCO 7KDa, Thermo Fisher) following manufacturer instructions. A 20-fold molar excess of NHS-PEG4-DBCO linker was added to buffer exchanged antibodies and incubated for 1 hr at room temperature. Excess linker was removed and antibody was purified using Zeba spin desalting column. DBCO modified antibodies were then coupled with azide fluorescent nanoparticle and incubated for 3 hours at 37° C. Antibody conjugated fluorescent nanoparticles were washed.

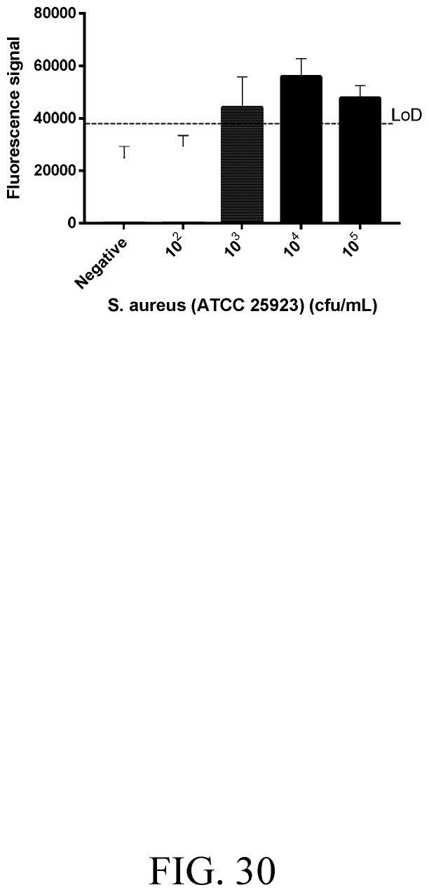

Blood samples were aggregated as previously described and spiked with a S. aureus (ATCC 25923) concentration range of 10²-10⁵ cfu/mL (n=3). Van-NPs (2 μL) and anti-S. aureus antibody-fluorescent NPs (1 μL) were added to spiked blood samples and incubated for 1.5 hours to label capture bacteria by forming a sandwich (FIG. 29). Nanoparticles bound bacteria were then washed and resuspended in 100 μL PBSP and fluorescence was measured on on a Tecan M 1000 Pro plate reader at excitation of 365 and emission of 416 nm, using a negative control of unspiked samples (FIG. 30). LoD (limit of detection) was calculated by the following equation:

LoD=N+(3×SD).

### Example 6. Synthesis and Assessment of Vancomycin Dimers

FIG. 32 provides a schematic illustration of synthesis of vancomycin-linker-vancomycin dimers of the invention. N₃-PEG3-Van (4) compounds (as hereinabove described) were considered particularly desirable for preparation of vancomycin dimers as the PEG linker may help to improve hydrophilicity compared to alkyl linkers. In order to generate the vancomycin dimer, a further bis-alkyne moiety (20) was utilised to connect two N₃-PEG3-Van compounds together. To produce the bis-alkyne moiety (20) adipic acid (19) was coupled with propargylamine in the presence of HCTU and DIPEA in DMF. The bis-alkyne linker (20) was then reacted with an excess of N₃-PEG3-Van by CuAAC using CuSO₄, NaASb and HOAc as catalyst in DMF/t-BuOH/H₂O under microwave reaction at 50° C. for 30-60 min to yield the vancomycin dimer (21a). Additionally, as further depicted in FIG. 32, a similar process was used to produce dimer (21b) using N₃-C8-Van (3) compounds (as hereinabove described).

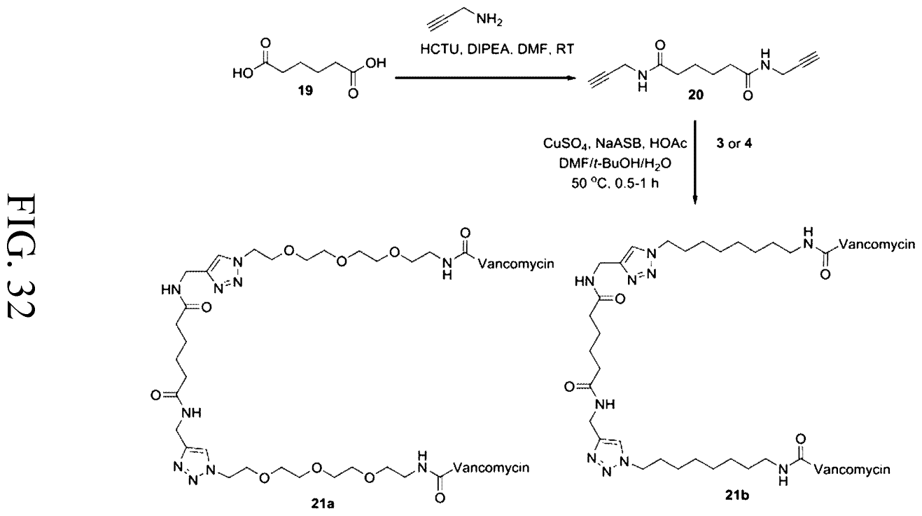

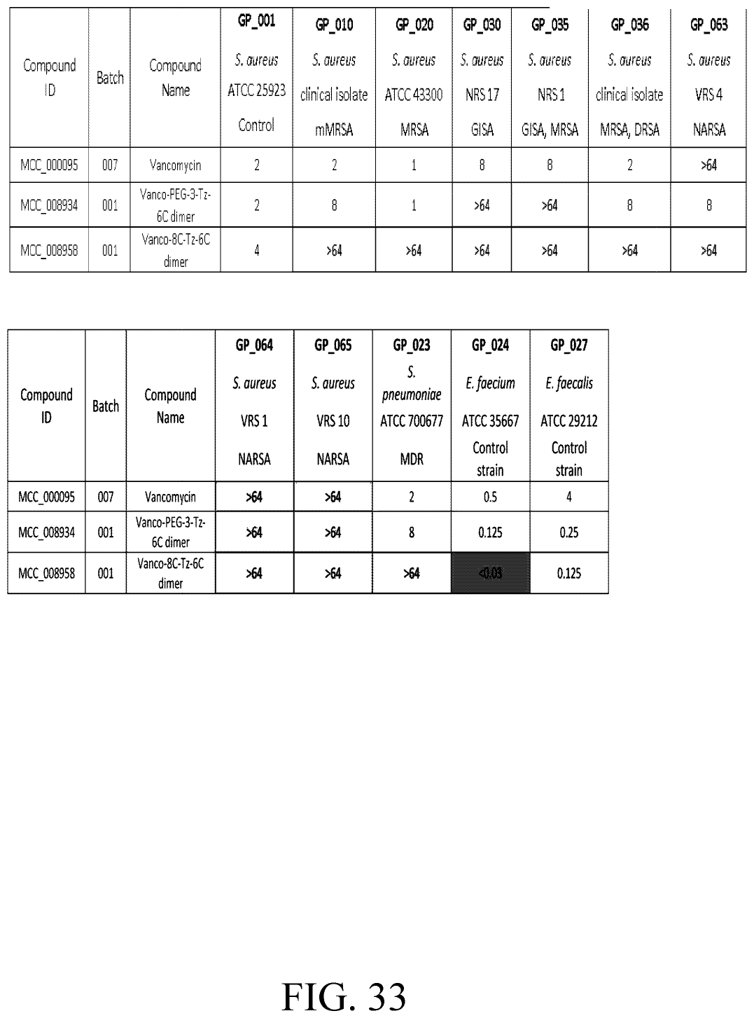

MIC of dimers 21a and 21b (and free vancomycin as a control) was assessed for various Gram positive bacterial species and strains, as depicted in FIG. 33. In FIG. 33, N₃-PEG3-Van dimer (21a) is labelled as ‘Vanco-3PEG-Tz-6C dimer’, and N₃-C8-Van dimer (21b) is labelled as Vanco-8C-Tz-6C dimer. The protocol used for the MIC assessment according to this example was as follows. Bacterial strains were cultured in Mueller Hinton broth (MHB) (Bacto laboratories, Cat. no. 211443) at 37° C. overnight. The culture was then diluted 40-fold in fresh MHB broth and incubated at 37° C. for 2-3 hrs. The compound was serially diluted two-fold across the wells of NBS 96-well micro-titre plates (Corning 3461 non-binding surface), with concentrations ranging from 0.06 μg/mL to 128 μg/mL, plated in duplicate. The resultant mid-log phase culture was diluted to 1×10⁶ CFU/mL, then 50 μL was added to each well of the compound-containing 96-well plates giving a cell density of 5×10⁵ CFU/mL, and a final compound concentration range of 0.03 μg/mL to 64 μg/mL. The plates were covered and incubated at 37° C. for 22 h. MICs were determined visually, being defined as the lowest concentration showing no visible growth. All each duplicated assessment was performed twice, such that total replication was quadruplicate. A positive control of a row of just the bacteria and a negative control of only the media was included for every plate tested.

Differences in activity among free vancomycin and dimers 21a and 21b were observed. Notably, increased activity (i.e. decreased MIC) of dimer 21a (but not dimer 21b) against vancomycin resistant S. aureus VRS4 NARSA was observed (FIG. 33). Additionally, both dimer 21a and dimer 21b showed increased activity (decreased MIC) against vancomycin sensitive E. faecium (strain ATCC 35667) and E. faecalis (strain ATCC 29212) (FIG. 33). This indicates that dimers of the invention may have particular advantages for antimicrobial use under at least certain circumstances.

### Example 7. Conjugation of Glycopeptide-Linker Adducts Using Polymer

In addition to the HSA-based conjugation approach set forth in Example 1, conjugation of N3-PEG3-Van to magnetic nanoparticles has also been performed using a process following that set out in PCT/AU2015/050564 (illustrated in FIG. 34).

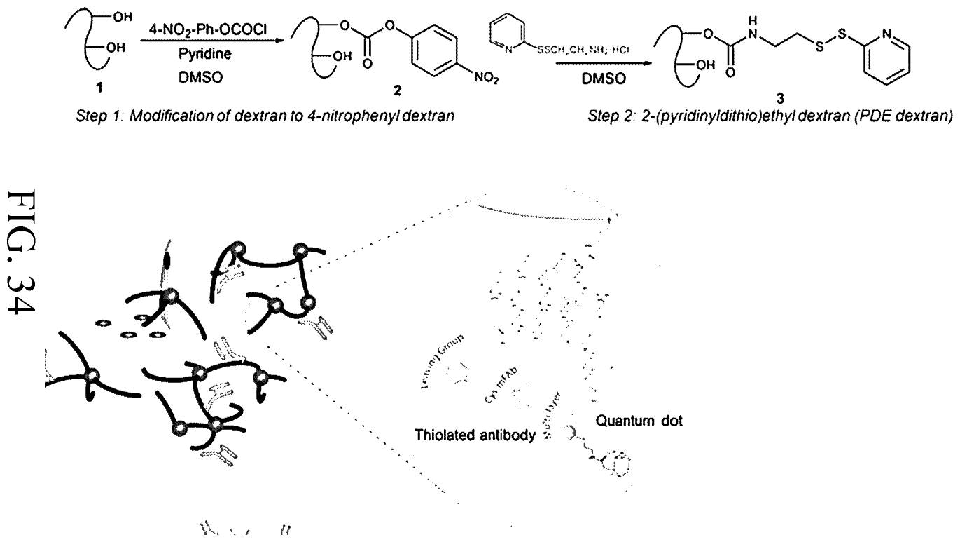

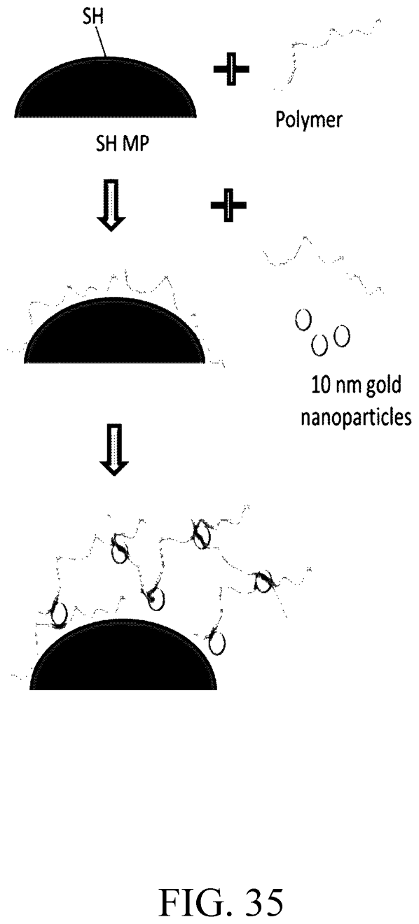

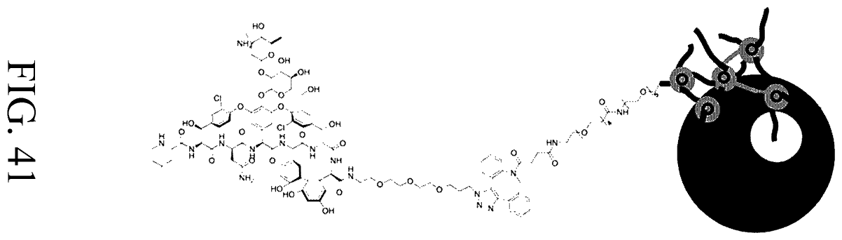

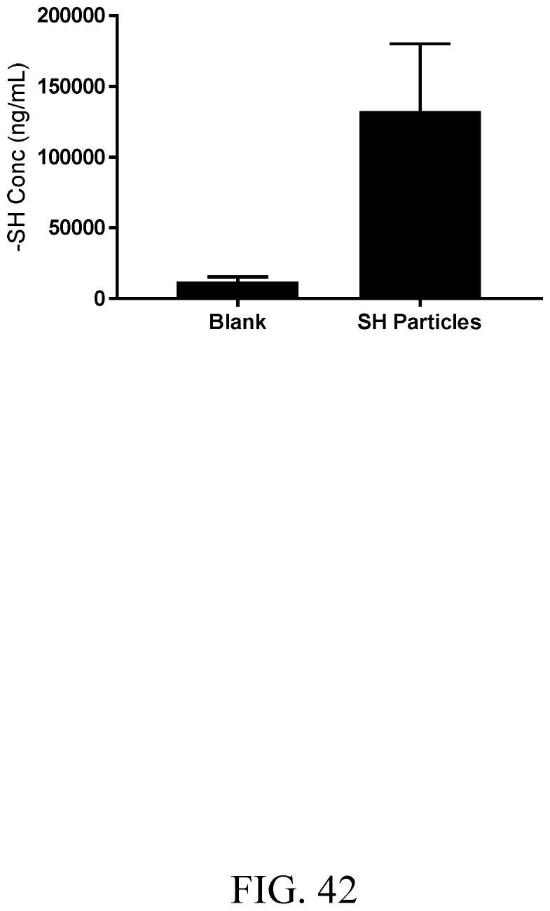

As set forth in as set forth in FIG. 35, PDEA dextran polymer was reacted with thiolated magnetic nanoparticles to form a monolayer on the nanoparticle surface. Nano-gold (741957, Sigma Aldrich) and additional PDEA dextran polymer was then reacted to form a polymer multilayer. Subsequently, N3-PEG3-Van was conjugated to this polymer coated nanoparticle similar as described above and in [7]. A schematic depiction of N3-PEG3-Van conjugated to magnetic nanoparticles using this polymer approach is provided in FIG. 41.

### Materials and Methods

Materials Required
MP: EMD Millipore, M1-020/50
Thiol PEG Amine (PG2-AMTH-5k, Nanocs)
NHS- PEG-DBCO, ClickChemistryTools #AZ103
Buffer A: CHES, 100 mM, pH 9, 0.1% Pluronic F127.
Buffer B: 100 mM MES pH 5, 0.01% Pluronic F127
Buffer C: 10 mM PBS pH 7.4, 0.1% Pluronic F127
Cystamine dihydrochloride, C121509 ALDRICH
Gold nanoparticles (741957, Sigma Aldrich)
EDC (1-ethyl-3-(3-dimethylaminopropyl)carbodiimide hydrochloride), 77149, ThermoFisher Scientific
Sulfo-NHS (N-hydroxysulfosuccinimide), 24510, ThermoFisher Scientific

Methods

1. Thiolation of COOH MNPs

Dissolve 50 mg of Cystamine in 0.5 mL of buffer C

Place 0.5 mL of MP in a low-protein binding eppendorf tubes. Wash twice in buffer B. Sonicate hard in a sonic bath

Add 6.25 mg of EDC and 6.25 mg of Sulfo-NHS to each eppendorf tube. Both compounds are moisture sensitive, therefore are aliquoted in glovebox and stored parafilmed in the freezer/fridge. Just before use, dilute them in cold buffer B (on ice) at 100 mg/mL and add 62.5 μL to the particles. IMPORTANT: put first Sulfo-NHS, then EDC. Sonicate for at least 2 minutes

Incubate 15 minutes at room temperature on orbital shaker.

Rapidly wash ×3 in Buffer A

Add 500 μL of previously prepared Cystamine and incubate 3 hrs at 4° C.

Wash three times in buffer A and resuspend in final volume of 0.5 mL

Estimate the thiol generated in the surface by Ellman's reaction

1.1 Procedure for Thiol Estimation on the Surface of MNPs Using Ellman's Reaction
Add 5 μL of thiolated MNPs to 250 μL of 10 mM Tris at pH 7.5 and 5 μL of Ellman's reagent (10 mM in Tris-buffer).
Incubate for 30 Mins at RT, read the OD of supernatant at 412 nm after magnetically pulling down the MNPs.
Quantify thiols by comparison to a standard curve made with cysteamine hydrochloride.

2. Generation of Multilayer of Polymer

Add 1 μL of PDEA polymer at 13 mg/mL

Incubate 1 hr, RT

Add 10 μL of gold nanoparticles (10 nm; 741957, Sigma Aldrich)

Incubate O/N in 4° C.

Magnetically pull down the MNPs and keep aside the supernatant for qualitative estimation of gold nanoparticles incorporation (2.1, below)

Wash three times in buffer C and resuspend in final volume of 0.5 mL

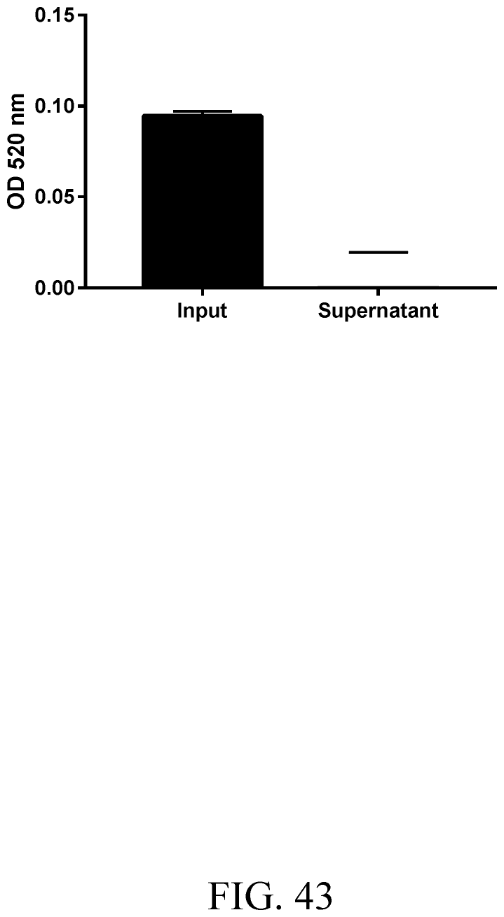

2.1 Gold Nanoparticles Used for this Project has an Absorption Peak at 520 nm
Take 50 μL of supernatant from the incorporation step and compare it with equal volume of nanogold added during multilayer generation.
Depletion of nano gold on the supernatant compared to input signifies successful incorporation of nanogold on the polymer to form a multilayer on the surface (See, FIG. 43).

3. PG2-AMTH-5k

PG2-AMTH-5k is aliquoted at 100 mg/mL in DMSO and stored at −20° C.

Add 12.5 μL of PG2-AMTH-5k to each eppendorf tube. Incubate 1 h at RT and wash three times in buffer C. Final volume 0.5 mL

4. NHS-PEG-DBCO

AZ103 is aliquoted at 100 mg/mL in DMSO and stored at −20° C.

Add 12.5 μL of AZ103 to each eppendorf tube. Incubate lh at RT and wash three times in buffer C. Final volume 0.5 mL

5. Cu Free Click of Vancomycin PEG N₃

Stock solution of Vancomycin PEG N₃ prepared at 10 mg/mL in 100% DMSO and stored at −20° C.

Add 20 μL of Vancomycin PEG N₃ to 150 μL of MP at 50 mg/mL. React for 4 h at 37° C.

Wash ×3 in Buffer C

### Results

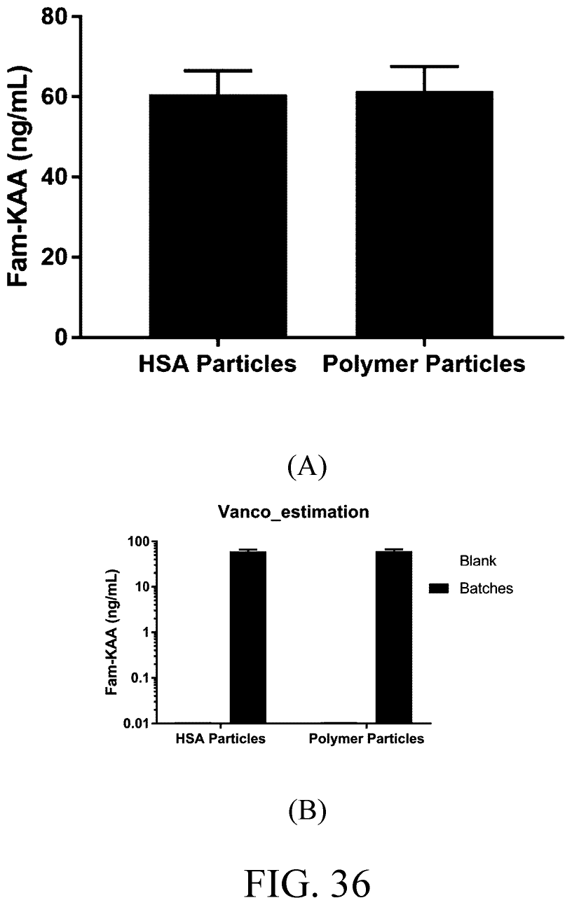

The concentration of vancomycin on the surface of nanoparticles coated with HSA and using the polymer approach was compared using the Fam-Kaa binding approach described in Example 1 and [8]. As seen in FIG. 36, the estimated surface concentration was substantially the same in each case. It was, however, noted that background (non-specific) binding to the coated microparticles was lower for those produced using the polymer approach (FIG. 36(B)).

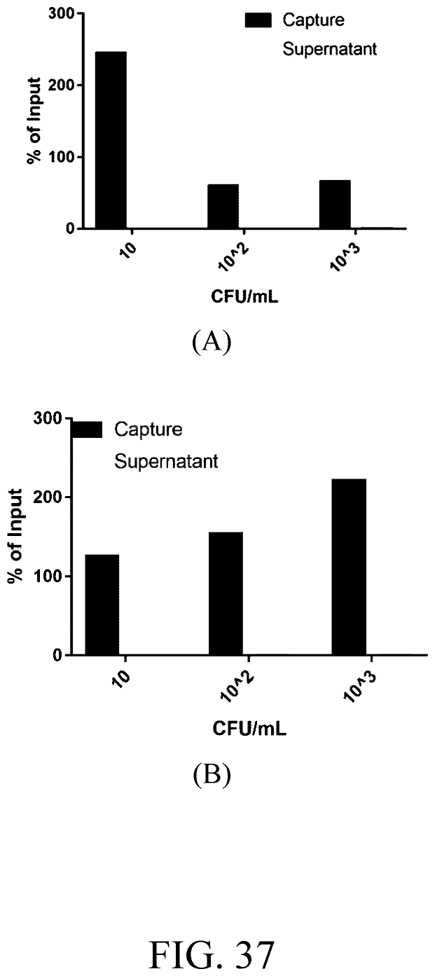

Additionally, bacterial capture (Staphylococcus aureus) using nanoparticles produced using the polymer and HSA approaches was compared. Both types of particles were successful in selective enrichment of S. aureus, as seen in FIG. 37.

### Example 8. Comparison of Glycopeptide Surface Concentration and Bacterial Capture Using C3, C8, and PEG Linkers

In addition to the constructs described in Example 1, further constructs were produced in a similar manner, but incorporating C3 or C8 in place of PEG3 in the first linker.

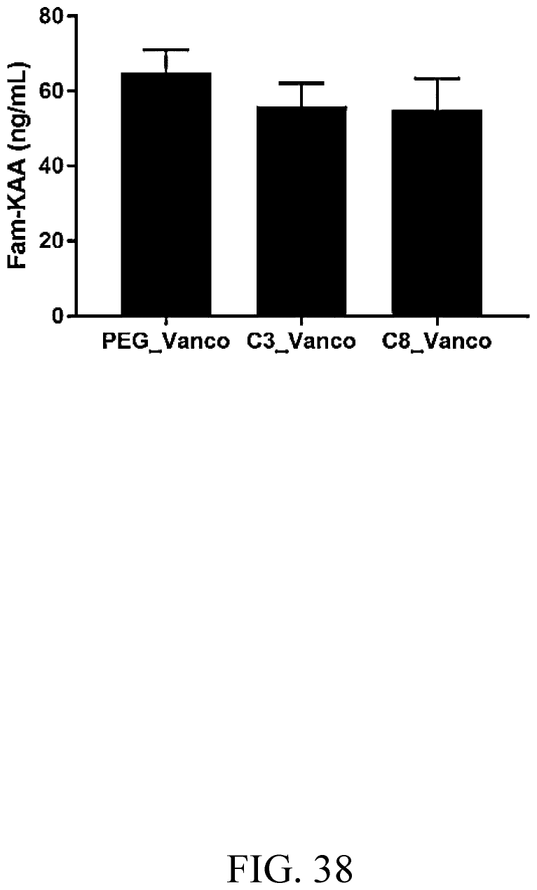

Assessment of the vancomycin surface concentration of these respective particles was performed using Fam-Kaa binding as set out in Example 1. As seen in FIG. 38, under the same conditions including substantially the same vancomycin concentrations. The use of the PEG3-containing linker resulted in a higher surface concentration of vancomycin than that of the C3 and C8 -containing linkers.

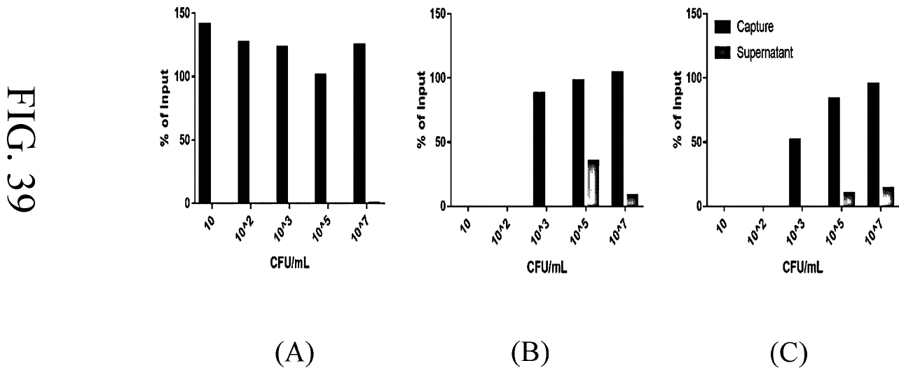

Furthermore, capture of gram positive bacteria (S. aureus GP01), as described above, using the PEG3, C8, and C3 constructs was assessed. As seen in FIG. 39, all constructs successfully captured bacteria from buffer (PBS pH 7.4) at concentrations of 10³-10⁷ cfu/mL spiked cells. However, only the PEG3 construct was observed to capture bacteria from lower concentrations of bacterial input (10 and 10² cfu/mL).

Additionally, there was a substantial difference in the number of bacterial present in the supernatant after capture. More specifically, very little or no bacteria was identified in the supernatant for the PEG3 constructs (A), while a substantial amount of bacteria was identified in the supernatant for the C8 (B) and C3 (C) constructs.

Without being bound by theory, it is hypothesized that the observed superior bacterial capture by the PEG3 constructs is related to the increased surface concentration of vancomycin of these constructs.

### Example 9. Antimicrobial Activity of Vancomycin Adducts

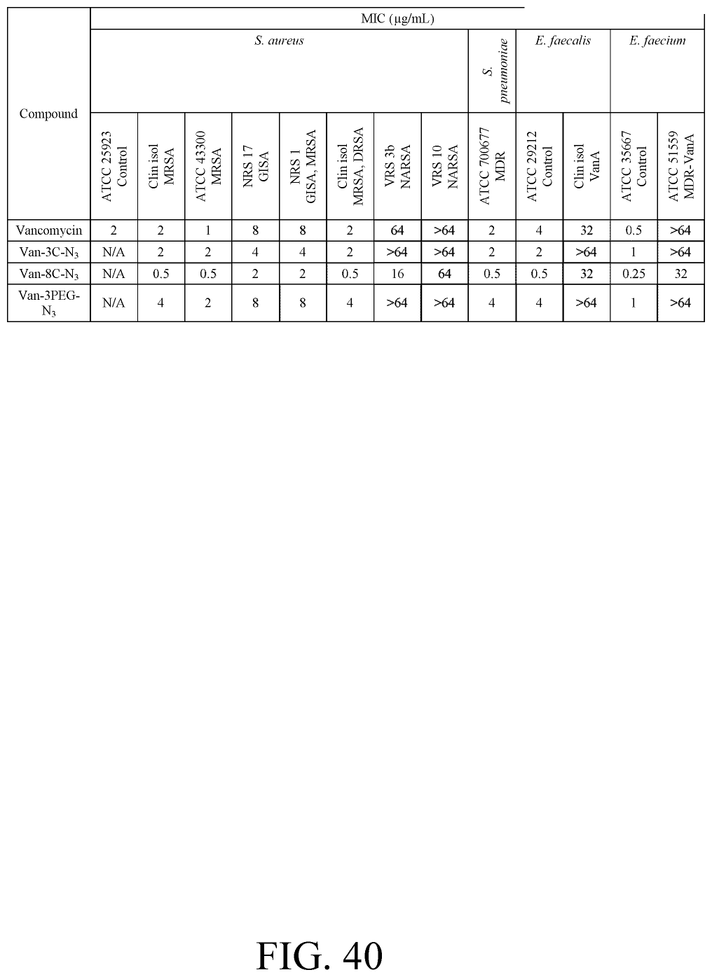

In addition to N3-PEG3-Van adducts, N3-C3-Van and N3-C8-Van adducts were synthesized similarly as described in Example 1. Antimicrobial activity in the form of Minimum Inhibitory Concentration of vancomycin and these adducts was then assessed and compared similar as described in Example 3. Results are set out in FIG. 40.

As can be seen in FIG. 40, N3-C8-Van adducts showed increased activity (in the form of decreased MIC) as compared to vancomycin against several Gram positive bacterial stains (including S. aureus MRSA strains, and S. pneumoniae, E. faecalis, and E. faecium strains).

Throughout the specification, the aim has been to describe the preferred embodiments of the invention without limiting the invention to any one embodiment or specific collection of features. Various changes and modifications may be made to the embodiments described and illustrated without departing from the present invention.

The disclosure of each patent and scientific document, computer program and algorithm referred to in this specification is incorporated by reference in its entirety.

### REFERENCES

1. Sanjaya, K. C., Ranzoni, A., Watterson, D., Young, P. & Cooper, M. A. Evaluation of direct versus multi-layer passivation and capture chemistries for nanoparticle-based bio sensor applications. Biosensors & bioelectronics 67, 769-774 (2015).
2. Eleaume, H., Jabbouri, S. Comparison of two standardisation methods in real-time quantitative RT-PCR to follow Staphylococcus aureus genes expression during in vitro growth. Journal of microbiological methods 59, 363-370 (2004).
3. Gevensleben, H., Garcia-Murillas, I., Graeser, M. K., Schiavon, G., Osin, P., Parton, M., Smith, I. E., Ashworth, A., Turner, N. C. Noninvasive detection of HER2 amplification with plasma DNA digital PCR. Clinical cancer research: an official journal of the American Association for Cancer Research 19, 3276-3284 (2013).
4. Cooper, M., Williams, D. H. & Cho, R. Y. Surface plasmon resonance analysis of glycopeptide antibiotic activity at a model membrane surface. Chemical Communications, 1625-1626 (1997).
5. Williams, S. C. et al. Distinguishing between living and nonliving bacteria: Evaluation of the vital stain propidium iodide and its combined use with molecular probes in aquatic samples. Journal of Microbiological Methods 32, 225-236 (1998).
6. Boulos, L., Prevost, M., Barbeau, B., Coallier, J. & Desjardins, R. LIVE/DEAD® BacLight™: application of a new rapid staining method for direct enumeration of viable and total bacteria in drinking water. Journal of Microbiological Methods 37, 77-86 (1999).
7. Hassan, M. M., Ranzoni, A. & Cooper M. A. A nanoparticle-based method for culture-free bacterial DNA enrichment from whole blood. Biosensors and Bioelectronics 99, 150-155 (2018).
8. Hassan, M. M., Ranzoni, A., Phetsang, W., Blaskovich, M. A. T. & Cooper, M. A. Surface Ligand Density of Antibiotic-Nanoparticle Conjugates Enhances Target Avidity and Membrane Permeabilization of Vancomycin-Resistant Bacteria. Bioconjugate Chemistry 28, 353-361 (2017).

### Tables

TABLE 1
Comparison of nanoparticle concentration and vancomycin density to MIC
for S. aureus strains.

| | Density of | MNPs | |
|---|---|---|---|
| MIC | vancomycin | Concentration | |
| (μg/mL) | per μm² | mg/mL | Strain |
| 0.55 | 2.18 × 10\hat{ }2 | 25 | S. aureus strain (ATCC 25923) |
| 3.3 | 2.41 × 10\hat{ }3 | 15 | S. aureus strain (ATCC 25923) |
| 4.5 | 3.94 × 10\hat{ }3 | 12.5 | S. aureus strain (ATCC 25923) |
| 13.3 | 6.25 × 10\hat{ }3 | 25 | S. aureus strain (NARSA |
| | | | VRS-1) |
| 15.2 | 9.56 × 10\hat{ }3 | 17.5 | S. aureus strain (NARSA |
| | | | VRS-1) |

1-53. (canceled)

54. A construct comprising:
(i) an optionally derivatized glycopeptide antibiotic;
(ii) a nanoparticle; and
(iii) a first linker connecting (i) and (ii).

55. The construct of claim 54, further comprising a second linker located between the first linker and (ii).

56. The construct of claim 54, wherein the nanoparticle is a separation nanoparticle, preferably wherein the separation nanoparticle is a magnetic nanoparticle.

57. The construct of claim 54, wherein the glycopeptide antibiotic is selected from the group consisting: of vancomycin; teicoplanin; oritavancin; telavancin; chloroeremomycin; and balhimycin.

58. The construct of claim 54, wherein the first linker comprises a polyethylene glycol (PEG) moiety.

59. The construct of claim 54, wherein the first linker comprises a linear C8 carbon chain.

60. The construct of claim 54, wherein the first linker is connected to the glycopeptide antibiotic via an amide bond.

61. The construct of claim 54, wherein the nanoparticle of the construct is passivated with a protein or a polymer.

62. A method of binding a construct to a microorganism or component thereof, the construct comprising (i) an optionally derivatized glycopeptide antibiotic; (ii) a nanoparticle; and (iii) a linker connecting (i) and (ii), the method including the steps of:
(a) contacting the construct and a microorganism or component thereof; and
(b) selectively binding the glycopeptide antibiotic of the construct with the microorganism or component thereof,
to thereby bind the construct to the microorganism or component thereof.

63. The method of claim 62 further including the steps of:
(c) selectively obtaining the construct bound to the microorganism or component thereof from a sample comprising the microorganism or component thereof using the nanoparticle, (d) separating the microorganism or component thereof from the sample using the construct.

64. The method of claim 63, wherein the sample is urine, blood, a blood sample, aggregating blood cells, or a blood product.

65. The method of claim 63, wherein the sample is obtained from a human.

66. The method of claim 62, wherein the microorganism is a Gram positive bacteria.

67. The method of claim 66, wherein the microorganism is pathogenic.

68. The method of claim 63, wherein step (c) is performed using magnetic capture of a separation nanoparticle that is a magnetic nanoparticle.

69. A method of screening a sample for the presence of a microorganism or component thereof of interest, the method including the steps of:
(a) combining a construct with a sample, the construct comprising (i) an optionally derivatized glycopeptide antibiotic; (ii) a nanoparticle; and (iii) a first linker connecting (i) and (ii);
(b) selectively obtaining the construct from the sample using the nanoparticle; and
(c) determining if a microorganism or component thereof of interest is or was bound to the construct,
wherein a determination that the microorganism or component thereof of interest is or was bound to the construct indicates that the sample contains the microorganism or component thereof of interest, and a determination that the microorganism or component thereof of interest is or was not bound to the construct indicates that the sample does not contain the microorganism or component thereof of interest.

70. A method of analysing a microorganism or component thereof obtained from a sample, the method including the steps of:
(a) combining a construct with a sample containing a microorganism or component thereof, the construct comprising (i) an optionally derivatized glycopeptide antibiotic; (ii) a nanoparticle; and (iii) a linker connecting (i) and (ii);
(b) selectively binding the microorganism or component thereof to the glycopeptide antibiotic of the construct;
(c) selectively obtaining the construct bound to the microorganism or component thereof from the sample using the nanoparticle to thereby obtain the microorganism or component thereof from the sample;
(d) optionally, separating the microorganism or component thereof from the construct; and
(e) analysing the microorganism.

71. The method of claim 69, wherein step (c) comprises MALDI mass spectrometry analysis, or nucleic acid analysis, or fluorescent analysis.

72. The method of claim 69, wherein step (c) comprises identification of the microorganism.

73. The method of claim 72, wherein the sample is a biological sample obtained from a subject, and wherein the method further includes the step of diagnosing a disease, disorder or condition in the subject based on the identity of the microorganism.

74. A method of inhibiting, controlling, or killing a microorganism, the method including the step of contacting the construct of claim 54 with a microorganism, to thereby inhibit, control, or kill the microorganism.
